(19) United States
(12) Patent Application Publication
(10) Pub. No.: US 2024/0083903 A1
(43) Pub. Date: Mar. 14, 2024

(54) ARYL ETHER COMPOUNDS AS TEAD MODULATORS

(71) Applicants: Jongwon Lim, Lexington, MA (US); Vladimir Simov, Boston, MA (US); Brandon A. Vara, Boston, MA (US); Charles S. Yeung, Boston, MA (US); Sebastian E. Schneider, Boston, MA (US); Erin F. DiMauro, Boston, MA (US); Merck Sharp& Dohme LLC, Rahway, NJ (US)

(72) Inventors: Jongwon Lim, Lexington, MA (US); Vladimir Simov, South Boston, MA (US); Brandon A. Vara, Boston, MA (US); Charles S. Yeung, Dedham, MA (US); Sebastian E. Schneider, Cambridge, MA (US); Erin F. DiMauro, Cambridge, MA (US)

(73) Assignee: Merck Sharp & Dohme LLC, Rahway, NJ (US)

(21) Appl. No.: 18/264,468

(22) PCT Filed: Feb. 15, 2022
(86) PCT No.: PCT/US2022/016378

§ 371 Date: Aug. 7, 2023

Related U.S. Application Data
(60) Provisional application No. 63/150,695, filed on Feb. 18, 2021.

(51) Int. Cl. C07D 487/04; A61P 35/00; C07D 209/34; C07D 215/20; C07D 217/04; C07D 231/56; C07D 235/24; C07D 235/26; C07D 237/28; C07D 239/84; C07D 263/57; C07D 265/36; C07D 277/66; C07D 317/56; C07D 471/04; C07D 495/04; C07D 498/04; C07D 513/04

(52) U.S. Cl. CPC C07D 487/04; A61P 35/00; C07D 209/34; C07D 215/20; C07D 217/04; C07D 231/56; C07D 235/24; C07D 235/26; C07D 237/28; C07D 239/84; C07D 263/57; C07D 265/36; C07D 277/66; C07D 317/56; C07D 471/04; C07D 495/04; C07D 498/04; C07D 513/04

(57) ABSTRACT

The present invention is directed to a compound of Formula I: or a pharmaceutically acceptable salt thereof, as well as pharmaceutical compositions comprising them and methods of using such compounds or pharmaceutical salts thereof.

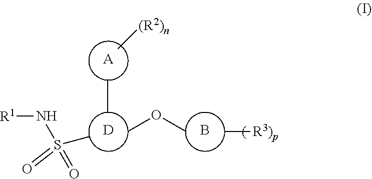

### BACKGROUND OF THE INVENTION

Yes-associated protein (YAP) and transcriptional co-activator with PDZ-binding motif (TAZ) are transcriptional co-activators of the Hippo pathway network and regulate cell proliferation, migration and apoptosis. Inhibition of the Hippo pathway promotes YAP/TAZ translocation to the nucleus, wherein YAP/TAZ interact with transcriptional enhancer associate domain (TEAD) transcription factors and coactivate the expression of target genes and promote cell proliferation. Hyperactivation of YAP and TAZ and/or mutations in one or more members of the Hippo pathway network have been implicated in numerous cancers. The instant invention described compounds that modulate the Hippo pathway. The compounds bind to the allosteric palmitate pocket of the TEAD1-4 transcription factors and thereby block the interaction between YAP1/TAZ and TEAD.

### SUMMARY OF THE INVENTION

The invention is directed to a compound of Formula I, or a pharmaceutical salt thereof, as well as pharmaceutical compositions comprising them and methods of using such compounds or pharmaceutical salts thereof. An embodiment of the invention provides a compound of Formula I:

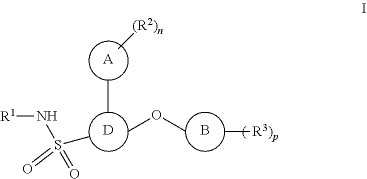

or a pharmaceutically acceptable salt thereof wherein;
Ring A is selected from a bicyclic ring structure:

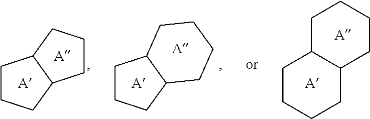

where the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring B is selected from phenyl, pyridinyl, 2,3-dihydro-1H-inden-5-yl or cyclohexyl;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R² is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆ alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆alkyl, OR, CF₃, or halo;
   where when R³ is C₄-C₆ alkyl, it can cyclize with Ring B to form a bicyclic ring;
n is selected from 0, 1, 2, 3 or 4;
p is selected from 1, 2 or 3; and
z is selected from 0, 1 or 2.

### DETAILED DESCRIPTION OF THE INVENTION

The present invention provides novel substituted aryl ether compounds, synthetic methods for making the compounds, pharmaceutical compositions containing them, isotopically-labeled compounds and methods of using the compounds as imaging agents.

In an embodiment, the present invention is directed to a compound of Formula I.

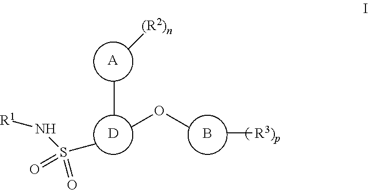

or a pharmaceutically acceptable salt thereof wherein;
Ring A is selected from

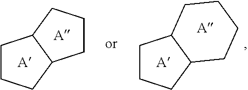 [SMILES: C1CC2CCCC2C1 ; C1CCC2CCCC2C1]

where the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring B is selected from phenyl, pyridinyl, 2,3-dihydro-1H-inden-5-yl or cyclohexyl;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R² is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆ alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆alkyl, OR, CF₃, or halo;
n is selected from 0, 1, 2, 3 or 4;
p is selected from 1, 2 or 3; and
z is selected from 0, 1 or 2.

Another embodiment of the invention provides a compound of Formula IA:

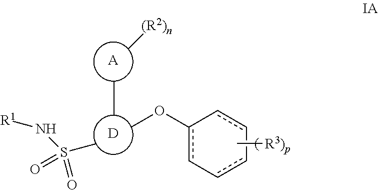

or a pharmaceutically acceptable salt thereof wherein;
---- represents a bond that may be present or absent;
Ring A is selected from a bicyclic ring structure:

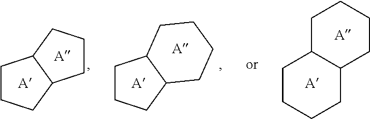

Wherein the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R²is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆ alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆alkyl, OR, CF₃, or halo;
n is selected from 0, 1, 2, 3 or 4;
p is selected from 1, 2 or 3; and
z is selected from 0, 1 or 2.

In a further embodiment of Formula I or Formula IA, Ring A is selected from dihydro-pyrrolo-pyrazolyl; dihydro-imidazo-oxazinyl; imidazo-pyridinyl; dihydro-pyrroloimidazolyl; imidazo-pyrazinyl; indazolyl; tetrahydroimidazo-pyridinyl; triazolopyrimidinyl; thiazolo-pyridinyl; benzimidazolyl; benzothiazolyl; dihydro-pyrrolo-thiazolyl; triazolo-pyrazinyl; tetrahydrothieno-pyridinyl; tetrahydroimidazo-pyrazinyl; imidazo-thiazolyl; pyrazolo-pyrimidinyl; dihydro-imidazo-oxazinyl; imidazo-pyridazinyl; imidazo-pyrimidinyl; benzothiazolyl; benzoxazolyl; pyrazolo-pyridinyl; tetrahydropyrazolo-pyridinyl; tetrahydroimidazo-pyridinyl; dihydroimidazo-oxazolyl; dihydro-imidazo-pyrazolyl; dihydropyrazolo-oxazolyl; cinnolinyl; dihydro-indolyl; dihydro-benzimidazolyl; indolyl; dihydro-benzimidazolyl; pyrrolo-pyridinyl; dihydro-isoindolyl; benzodioxolyl; indazolyl; dihydro-pyrazolo-oxazinyl; isoquinolinyl; quinolinyl; quinazolinyl; quinoxalinyl; dihydro-benzoxazinyl; tetrahydroquinolinyl; dihydro-benzoxazinyl; naphthyridinyl; dihydroquinazolinyl; dihydropyrazolooxazolyl; dihydropyrrolopyrazolyl; dihydrocyclopentapyrazolyl; dihydropyrrolopyrazolyl; dihydro-pyrroloimidazolyl; or azaspiroheptenyl; and all other substituents and variables are as defined above.

Representative compounds of the present invention include compounds selected from

| Ex. | | |
|---|---|---|
| No. | Structure | Name |
| 1-1 | 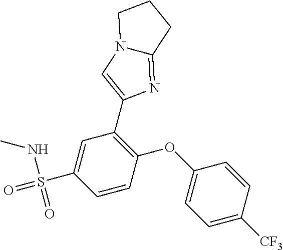 [SMILES: CNS(=O)(=O)c1ccc(Oc2ccc(C(F)(F)F)cc2)c(-c2cn3c(n2)CCC3)c1] | 3-(6,7-Dihydro-5H-pyrrolo[1,2-a] imidazol-2-yl)-N-methyl-4-(4- (trifluoromethyl)phenoxy)benzenesulfonamide |
| 1-2 | 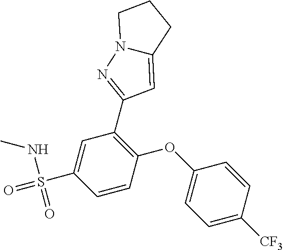 [SMILES: CNS(=O)(=O)c1ccc(Oc2ccc(C(F)(F)F)cc2)c(-c2cc3n(n2)CCC3)c1] | 3-(5,6-Dihydro-4H-pyrrolo[1,2-b] pyrazol-2-yl)-N-methyl-4-(4- (trifluoromethyl)phenoxy)benzenesulfonamide |
| 1-3 | 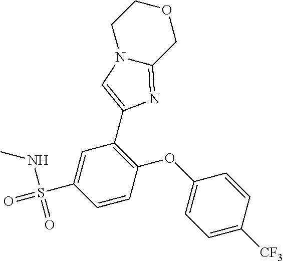 [SMILES: CNS(=O)(=O)c1ccc(Oc2ccc(C(F)(F)F)cc2)c(-c2cn3c(n2)COCC3)c1] | 3-(5,6-Dihydro-8H-imidazo[2,1-c] [1,4]oxazin-2-yl)-N-methyl-4-(4- (trifluoromethyl)phenoxy)benzenesulfonamide |
| 1-4 | 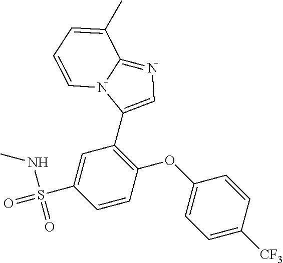 [SMILES: CNS(=O)(=O)c1ccc(Oc2ccc(C(F)(F)F)cc2)c(-c2cnc3c(C)cccn23)c1] | N-Methyl-3-(8-methylimidazo[1,2-a] pyridin-3-yl)-4-(4- (trifluoromethyl)phenoxy)benzenesulfonamide |
| 1-5 | 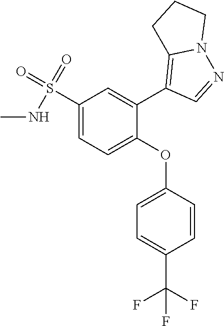 [SMILES: CNS(=O)(=O)c1ccc(Oc2ccc(C(F)(F)F)cc2)c(-c2cnn3c2CCC3)c1] | 3-(5,6-dihydro-4H-pyrrolo[1,2-b] pyrazol-3-yl)-N-methyl-4-[4- (trifluoromethyl)phenoxy]benzene-1-sulfonamide |
| 1-6 | 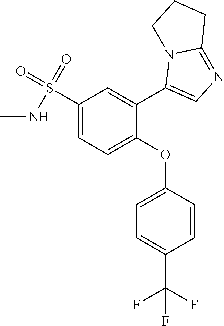 [SMILES: CNS(=O)(=O)c1ccc(Oc2ccc(C(F)(F)F)cc2)c(-c2cnc3n2CCC3)c1] | 3-(6,7-dihydro-5H-pyrrolo[1,2-a] imidazol-3-yl)-N-methyl-4-[4- (trifluoromethyl)phenoxy]benzene-1-sulfonamide |
| 1-7 | 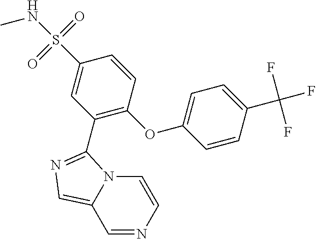 [SMILES: CNS(=O)(=O)c1ccc(Oc2ccc(C(F)(F)F)cc2)c(-c2ncc3cnccn23)c1] | 3-(imidazo[1,5-a]pyrazin-3-yl)- N-methyl-4-[4- (trifluoromethyl)phenoxy]benzene- 1-sulfonamide |
| 1-8 | 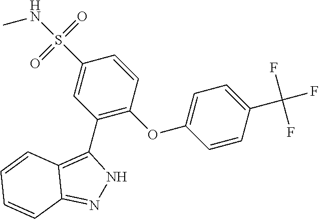 [SMILES: CNS(=O)(=O)c1ccc(Oc2ccc(C(F)(F)F)cc2)c(-c2[nH]nc3ccccc23)c1] | 3-(2H-indazol-3-yl)-N- methyl-4-[4- (trifluoromethyl)phenoxy]benzene- 1-sulfonamide |
| 1-9 | 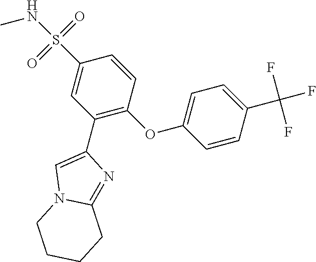 [SMILES: CNS(=O)(=O)c1ccc(Oc2ccc(C(F)(F)F)cc2)c(-c2cn3c(n2)CCCC3)c1] | N-methyl-3-(5,6,7,8-tetrahydroimidazo [1,2-a]pyridin-2-yl)-4-[4- (trifluoromethyl)phenoxy]benzene-1-sulfonamide |
| 1-10 | 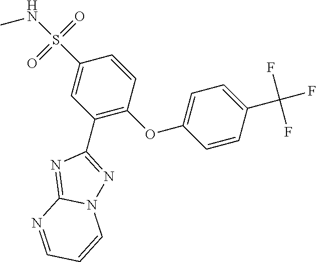 [SMILES: CNS(=O)(=O)c1ccc(Oc2ccc(C(F)(F)F)cc2)c(-c2nc3ncccn3n2)c1] | N-methyl-3-([1,2,4]triazolo[1,5-a] pyrimidin-2-yl)-4-[4- (trifluoromethyl)phenoxy]benzene-1-sulfonamide |
| 1-11 | 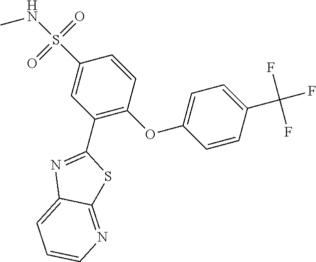 [SMILES: CNS(=O)(=O)c1ccc(Oc2ccc(C(F)(F)F)cc2)c(-c2nc3cccnc3s2)c1] | N-methyl-3-([1,3]thiazolo[5,4-b] pyridin-2-yl)-4-[4- (trifluoromethyl)phenoxy]benzene-1-sulfonamide |
| 1-12 | 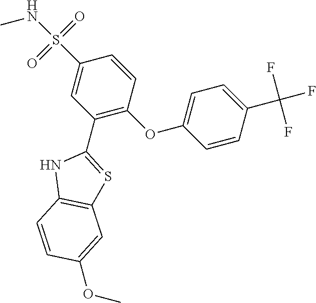 | 3-(5-methoxy-1H-benzimidazol-2-yl)- N-methy1-4-[4- (trifluoromethyl)phenoxy]benzene- 1-sulfonamide |
| 1-13 | 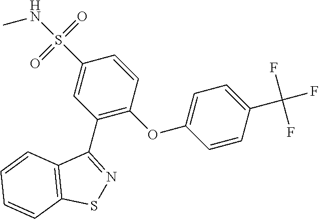 [SMILES: CNS(=O)(=O)c1ccc(Oc2ccc(C(F)(F)F)cc2)c(-c2nsc3ccccc23)c1] | 3-(1,2-benzothiazol-3-yl)-N- methyl-4-[4- (trifluoromethyl)phenoxy]benzene- 1-sulfonamide |
| 1-14 | 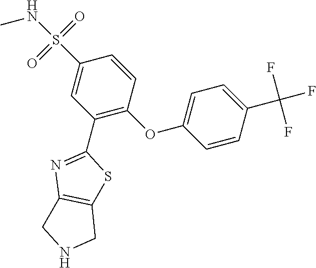 [SMILES: CNS(=O)(=O)c1ccc(Oc2ccc(C(F)(F)F)cc2)c(-c2nc3c(s2)CNC3)c1] | 3-(5,6-dihydro-4H-pyrrolo[3,4-d] [1,3]thiazol-2-yl)-N-methyl-4-[4- (trifluoromethyl)phenoxy]benzene-1-sulfonamide |
| 1-15 | 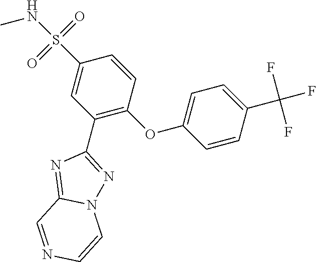 [SMILES: CNS(=O)(=O)c1ccc(Oc2ccc(C(F)(F)F)cc2)c(-c2nc3cnccn3n2)c1] | N-methyl-3-([1,2,4]triazolo[1,5-a] pyrazin-2-yl)-4-[4- (trifluoromethyl)phenoxy]benzene-1-sulfonamide |
| 1-16 | 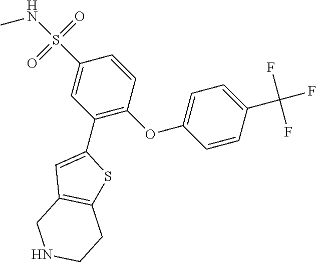 [SMILES: CNS(=O)(=O)c1ccc(Oc2ccc(C(F)(F)F)cc2)c(-c2cc3c(s2)CCNC3)c1] | N-methyl-3-(4,5,6,7-tetrahydrothieno[3,2-c] pyridin-2-yl)-4-[4- (trifluoromethyl)phenoxy]benzene-1-sulfonamide |
| 1-17 | 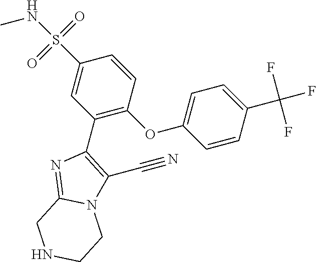 [SMILES: CNS(=O)(=O)c1ccc(Oc2ccc(C(F)(F)F)cc2)c(-c2nc3n(c2C#N)CCNC3)c1] | 3-(3-cyano-5,6,7,8-tetrahydroimidazo[1,2-a] pyrazin-2-yl)-N-methyl-4-[4- (trifluoromethyl)phenoxy]benzene-1-sulfonamide |
| 1-18 | 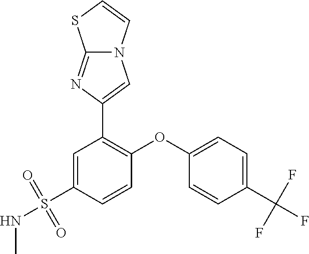 [SMILES: CNS(=O)(=O)c1ccc(Oc2ccc(C(F)(F)F)cc2)c(-c2cn3ccsc3n2)c1] | 3-(imidazo[2,1-b][1,3]thiazol-6-yl)- N-methyl-4-[4- (trifluoromethyl)phenoxy]benzene- 1-sulfonamide |
| 1-19 | 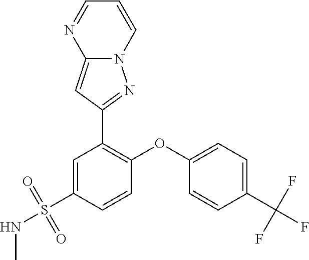 [SMILES: CNS(=O)(=O)c1ccc(Oc2ccc(C(F)(F)F)cc2)c(-c2cc3ncccn3n2)c1] | N-methyl-3-(pyrazolo[1,5-a] pyrimidin-2-yl)-4-[4- (trifluoromethyl)phenoxy]benzene- 1-sulfonamide |
| 1-20 | 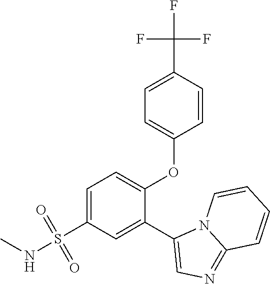 [SMILES: CNS(=O)(=O)c1ccc(Oc2ccc(C(F)(F)F)cc2)c(-c2cnc3ccccn23)c1] | 3-(imidazo[1,2-a]pyridin-3-yl)- N-methyl-4-[4- (trifluoromethyl)phenoxy]benzene- 1-sulfonamide |
| 1-21 | 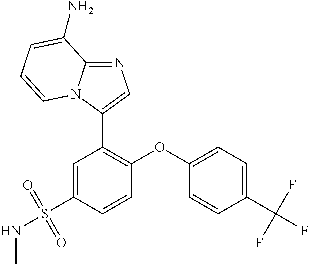 [SMILES: CNS(=O)(=O)c1ccc(Oc2ccc(C(F)(F)F)cc2)c(-c2cnc3c(N)cccn23)c1] | 3-(8-aminoimidazo[1,2-a]pyridin-3-yl)- N-methyl-4-[4- (trifluoromethyl)phenoxy]benzene- 1-sulfonamide |
| 1-22 | 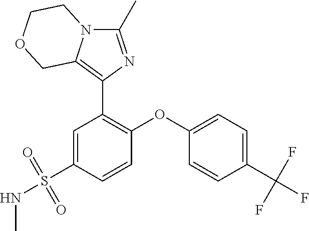 [SMILES: CNS(=O)(=O)c1ccc(Oc2ccc(C(F)(F)F)cc2)c(-c2nc(C)n3c2COCC3)c1] | N-methyl-3-(3-methyl-5,6-dihydro- 8H-imidazo[5,1-c][1,4]oxazin-1-yl)- 4-[4-(trifluoromethyl)phenoxy]benzene- 1-sulfonamide |
| 1-23 | 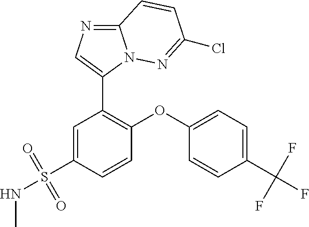 [SMILES: CNS(=O)(=O)c1ccc(Oc2ccc(C(F)(F)F)cc2)c(-c2cnc3ccc(Cl)nn23)c1] | 3-(6-chloroimidazo[1,2-b] pyridazin-3-yl)-N-methyl-4- [4-(trifluoromethyl)phenoxy]benzene- 1-sulfonamide |
| 1-24 | 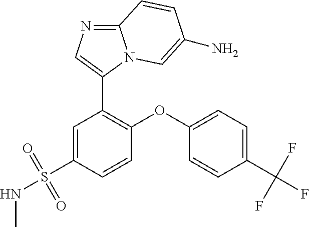 [SMILES: CNS(=O)(=O)c1ccc(Oc2ccc(C(F)(F)F)cc2)c(-c2cnc3ccc(N)cn23)c1] | 3-(6-aminoimidazo[1,2-a] pyridin-3-yl)-N-methyl-4- [4-(trifluoromethyl)phenoxy]benzene- 1-sulfonamide |
| 1-25 | 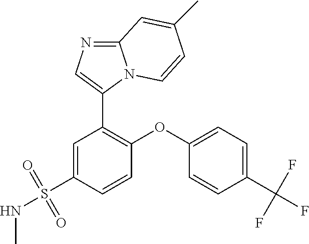 [SMILES: CNS(=O)(=O)c1ccc(Oc2ccc(C(F)(F)F)cc2)c(-c2cnc3cc(C)ccn23)c1] | N-methyl-3-(7-methylimidazo[1,2-a] pyridin-3-yl)-4- [4-(trifluoromethyl)phenoxy]benzene- 1-sulfonamide |
| 1-26 | 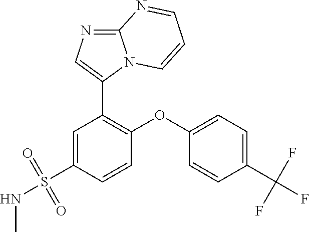 [SMILES: CNS(=O)(=O)c1ccc(Oc2ccc(C(F)(F)F)cc2)c(-c2cnc3ncccn23)c1] | 3-(imidazo[1,2-a]pyrimidin-3-yl)- N-methyl-4- [4-(trifluoromethyl)phenoxy]benzene- 1-sulfonamide |
| 1-27 | 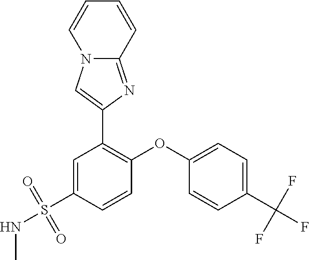 [SMILES: CNS(=O)(=O)c1ccc(Oc2ccc(C(F)(F)F)cc2)c(-c2cn3ccccc3n2)c1] | 3-(imidazo[1,2-a]pyridin-2-yl)-N- methyl-4- [4-(trifluoromethyl)phenoxy]benzene- 1-sulfonamide |
| 1-28 | 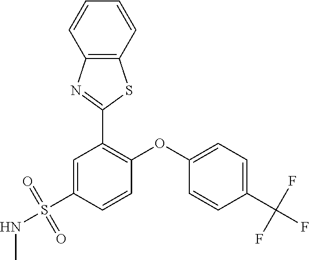 [SMILES: CNS(=O)(=O)c1ccc(Oc2ccc(C(F)(F)F)cc2)c(-c2nc3ccccc3s2)c1] | 3-(1,3-benzothiazol-2-yl)-N- methyl-4- [4-(trifluoromethyl)phenoxy]benzene- 1-sulfonamide |
| 1-29 | 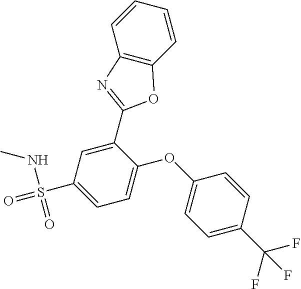 [SMILES: CNS(=O)(=O)c1ccc(Oc2ccc(C(F)(F)F)cc2)c(-c2nc3ccccc3o2)c1] | 3-(1,3-benzoxazol-2-yl)-N- methyl-4- [4-(trifluoromethyl)phenoxy]benzene- 1-sulfonamide |
| 1-30 | 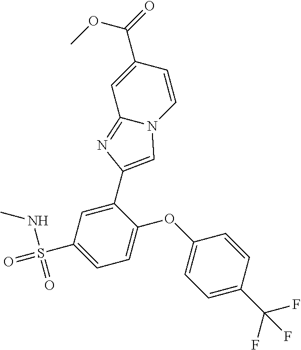 [SMILES: CNS(=O)(=O)c1ccc(Oc2ccc(C(F)(F)F)cc2)c(-c2cn3ccc(C(=O)OC)cc3n2)c1] | methyl 2-{5-(methylsulfamoyl)- 2-[4-(trifluoromethyl)phenoxy]phenyl} imidazo[1,2-a]pyridine-7-carboxylate |
| 1-31 | 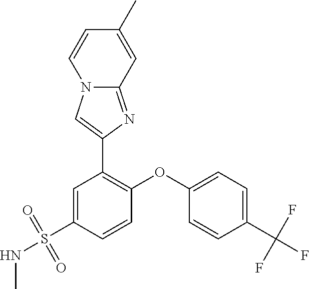 [SMILES: CNS(=O)(=O)c1ccc(Oc2ccc(C(F)(F)F)cc2)c(-c2cn3ccc(C)cc3n2)c1] | N-methyl-3-(7-methylimidazo[1,2-a] pyridin-2-yl)-4- [4-(trifluoromethyl)phenoxy]benzene- 1-sulfonamide |
| 1-32 | 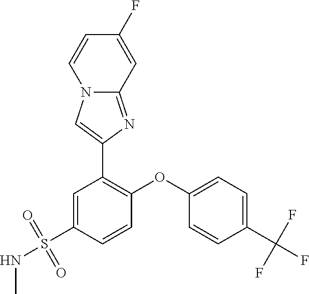 [SMILES: CNS(=O)(=O)c1ccc(Oc2ccc(C(F)(F)F)cc2)c(-c2cn3ccc(F)cc3n2)c1] | 3-(7-fluoroimidazo[1,2-a] pyridin-2-yl)-N-methyl-4- [4-(trifluoromethyl)phenoxy]benzene- 1-sulfonamide |
| 1-33 | 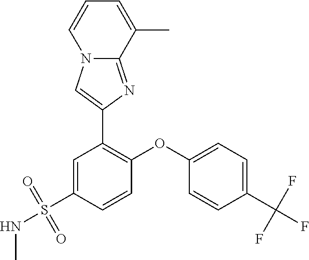 [SMILES: CNS(=O)(=O)c1ccc(Oc2ccc(C(F)(F)F)cc2)c(-c2cn3cccc(C)c3n2)c1] | N-methyl-3-(8-methylimidazo[1,2-a] pyridin-2-yl)-4- [4-(trifluoromethyl)phenoxy]benzene- 1-sulfonamide |
| 1-34 | 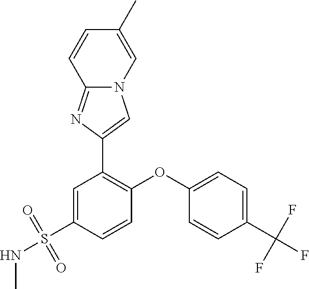 [SMILES: CNS(=O)(=O)c1ccc(Oc2ccc(C(F)(F)F)cc2)c(-c2cn3cc(C)ccc3n2)c1] | N-methyl-3-(6-methylimidazo[1,2-a] pyridin-2-yl)-4- [4-(trifluoromethyl)phenoxy]benzene- 1-sulfonamide |
| 1-35 | 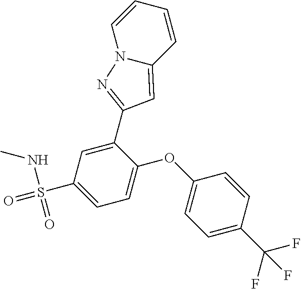 [SMILES: CNS(=O)(=O)c1ccc(Oc2ccc(C(F)(F)F)cc2)c(-c2cc3ccccn3n2)c1] | N-methyl-3-(pyrazolo[1,5-a] pyridin-2-y1)-4- [4-(trifluoromethyl)phenoxy]benzene- 1-sulfonamide |
| 1-36 | 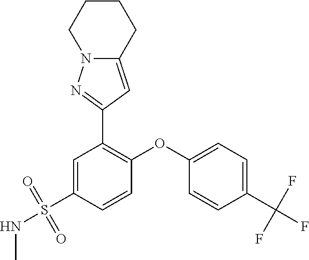 [SMILES: CNS(=O)(=O)c1ccc(Oc2ccc(C(F)(F)F)cc2)c(-c2cc3n(n2)CCCC3)c1] | N-methyl-3- (4,5,6,7-tetrahydropyrazolo[1,5-a] pyridin-2-yl)-4- [4-(trifluoromethyl)phenoxy]benzene- 1-sulfonamide |
| 1-37 | 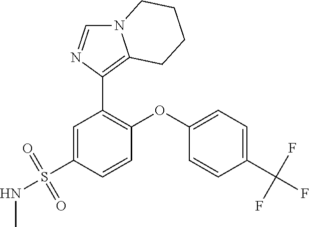 [SMILES: CNS(=O)(=O)c1ccc(Oc2ccc(C(F)(F)F)cc2)c(-c2ncn3c2CCCC3)c1] | N-methyl-3- (5,6,7,8-tetrahydroimidazo[1,5-a] pyridin-1-yl)-4- [4-(trifluoromethyl)phenoxy]benzene- 1-sulfonamide |
| 1-38 | 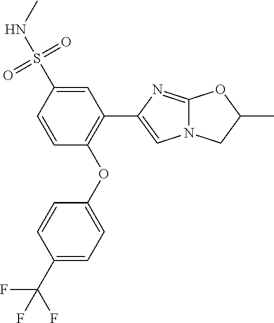 [SMILES: CNS(=O)(=O)c1ccc(Oc2ccc(C(F)(F)F)cc2)c(-c2cn3c(n2)OC(C)C3)c1] | (R)- or (S)-N-methyl-3- (2-methyl-2,3-dihydroimidazo[2,1-b] [1,3]oxazol-6-yl)-4- [4-(trifluoromethyl)phenoxy]benzene- 1-sulfonamide |
| 1-39 | 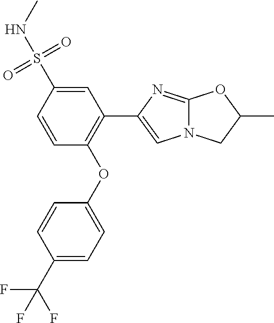 [SMILES: CNS(=O)(=O)c1ccc(Oc2ccc(C(F)(F)F)cc2)c(-c2cn3c(n2)OC(C)C3)c1] | (S)- or (R)-N-methyl-3- (2-methyl-2,3-dihydroimidazo[2,1-b] [1,3]oxazol-6-yl)-4- [4-(trifluoromethyl)phenoxy]benzene- 1-sulfonamide |
| 1-40 | 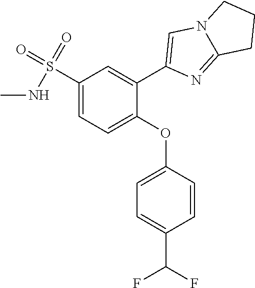 [SMILES: CNS(=O)(=O)c1ccc(Oc2ccc(C(F)F)cc2)c(-c2cn3c(n2)CCC3)c1] | 4-[4-(difluoromethyl)phenoxy]-3- (6,7-dihydro-5H-pyrrolo[1,2-a]imidazol- 2-yl)-N-methylbenzene-1-sulfonamide |
| 1-41 | 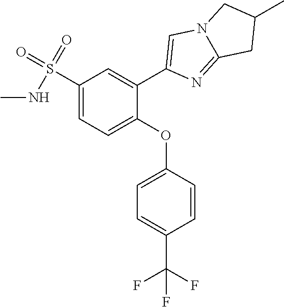 | (R)- or (S)-N-methyl-3- (6-methyl-6,7-dihydro-5H-pyrrolo [1,2-a]imidazol-2-yl)-4- [4-(trifluoromethyl)phenoxy]benzene- 1-sulfonamide |
| 1-42 | 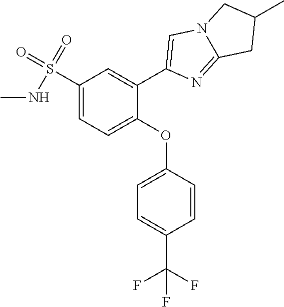 | (S)- or (R)-N-methyl-3- (6-methyl-6,7-dihydro-5H- pyrrolo[1,2-a]imidazol-2-yl)-4- [4-(trifluoromethyl)phenoxy]benzene- 1-sulfonamide |
| 1-43 | 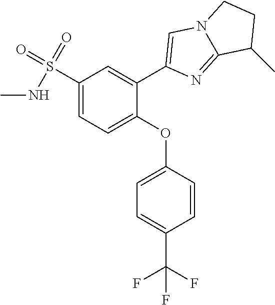 | (R)- or (S)-N-methyl-3- (7-methyl-6,7-dihydro-5H- pyrrolo[1,2-a]imidazol-2-yl)-4- [4-(trifluoromethyl)phenoxy]benzene- 1-sulfonamide |
| 1-44 | 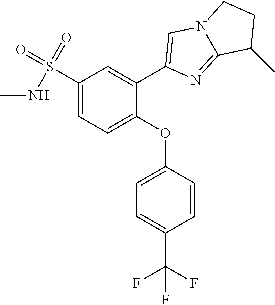 | (S)- or (R)-N-methyl-3- (7-methyl-6,7-dihydro-5H- pyrrolo[1,2-a]imidazol-2-yl)-4- [4-(trifluoromethyl)phenoxy]benzene- 1-sulfonamide |
| 1-45 | 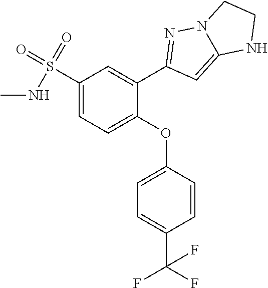 [SMILES: CNS(=O)(=O)c1ccc(Oc2ccc(C(F)(F)F)cc2)c(-c2cc3n(n2)CCN3)c1] | 3-(2,3-dihydro-1H- imidazo[1,2-b]pyrazol-6-yl)- N-methyl-4- [4-(trifluoromethyl)phenoxy]benzene- 1-sulfonamide |
| 1-46 | 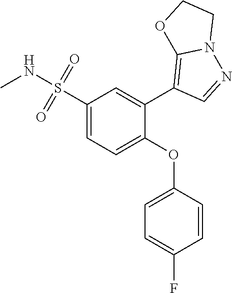 [SMILES: CNS(=O)(=O)c1ccc(Oc2ccc(F)cc2)c(-c2cnn3c2OCC3)c1] | 3-(2,3-dihydropyrazolo[5,1-b] oxazol-7-yl)-4- (4-fluorophenoxy)-N- methylbenzenesulfonamide |
| 1-47 | 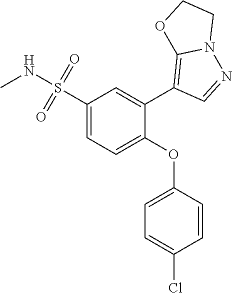 [SMILES: CNS(=O)(=O)c1ccc(Oc2ccc(Cl)cc2)c(-c2cnn3c2OCC3)c1] | 4-(4-chlorophenoxy)-3- (2,3-dihydropyrazolo[5,1-b] oxazol-7-yl)-N- methylbenzenesulfonamide |
| 1-48 | 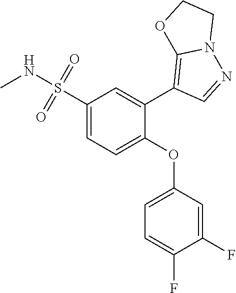 [SMILES: CNS(=O)(=O)c1ccc(Oc2ccc(F)c(F)c2)c(-c2cnn3c2OCC3)c1] | 4-(3,4-difluorophenoxy)- 3-(2,3-dihydropyrazolo[5,1-b] oxazol-7-yl)-N- methylbenzenesulfonamide |
| 1-49 | 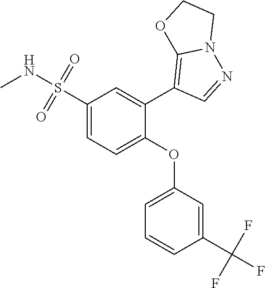 [SMILES: CNS(=O)(=O)c1ccc(Oc2cccc(C(F)(F)F)c2)c(-c2cnn3c2OCC3)c1] | 3-(2,3-dihydropyrazolo[5,1-b] oxazol-7-yl)-N-methyl-4-(3- (trifluoromethyl)phenoxy)benzenesulfonamide |
| 1-50 | 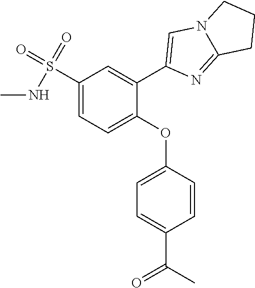 [SMILES: CNS(=O)(=O)c1ccc(Oc2ccc(C(C)=O)cc2)c(-c2cn3c(n2)CCC3)c1] | 4-(4-acetylphenoxy)-3- (6,7-dihydro-5H- pyrrolo[1,2-a]imidazol-2-yl)- N-methylbenzene-1-sulfonamide |
| 2-1 | 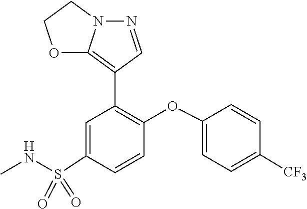 [SMILES: CNS(=O)(=O)c1ccc(Oc2ccc(C(F)(F)F)cc2)c(-c2cnn3c2OCC3)c1] | 3-(2,3-Dihydropyrazolo[5,1-b] oxazol-7-yl)-N-methyl-4- (4- (trifluoromethyl)phenoxy)benzenesulfonamide |
| 2-2 | 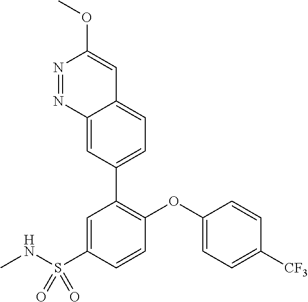 [SMILES: CNS(=O)(=O)c1ccc(Oc2ccc(C(F)(F)F)cc2)c(-c2ccc3cc(OC)nnc3c2)c1] | 3-(3-Methoxycinnolin-7-yl)-N- methyl-4-[4- (trifluoromethyl)phenoxy]benzene- 1-sulfonamide |
| 2-3 | 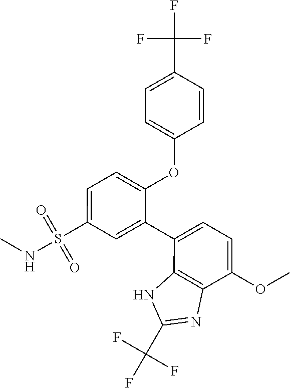 | 3-[4-methoxy-2-(trifluoromethyl)- 1H-benzimidazol-7-yl]-N-methyl-4- [4-(trifluoromethyl)phenoxy]benzene- 1-sulfonamide |
| 2-4 | 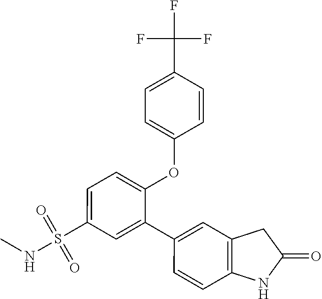 [SMILES: CNS(=O)(=O)c1ccc(Oc2ccc(C(F)(F)F)cc2)c(-c2ccc3c(c2)CC(=O)N3)c1] | N-methyl-3-(2-oxo-2,3-dihydro- 1H-indol-5-yl)-4-[4- (trifluoromethyl)phenoxy]benzene- 1-sulfonamide |
| 2-5 | 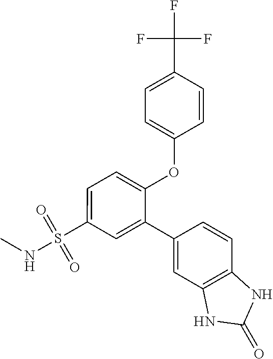 [SMILES: CNS(=O)(=O)c1ccc(Oc2ccc(C(F)(F)F)cc2)c(-c2ccc3[nH]c(=O)[nH]c3c2)c1] | N-methyl-3-(2-oxo-2,3-dihydro- 1H-benzimidazol-5-yl)-4-[4- (trifluoromethyl)phenoxy]benzene- 1-sulfonamide |
| 2-6 | 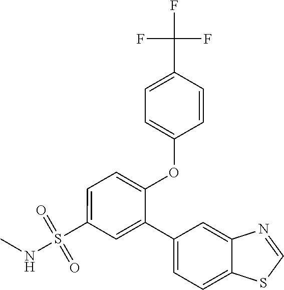 [SMILES: CNS(=O)(=O)c1ccc(Oc2ccc(C(F)(F)F)cc2)c(-c2ccc3scnc3c2)c1] | 3-(1,3-benzothiazol-5-yl)-N- methyl-4-[4- (trifluoromethyl)phenoxy]benzene- 1-sulfonamide |
| 2-7 | 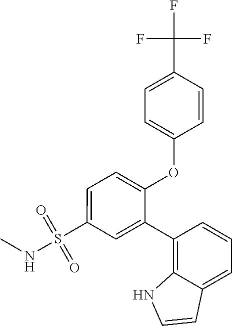 [SMILES: CNS(=O)(=O)c1ccc(Oc2ccc(C(F)(F)F)cc2)c(-c2cccc3cc[nH]c23)c1] | 3-(1H-indol-7-yl)-N-methyl-4- [4-(trifluoromethyl)phenoxy]benzene- 1-sulfonamide |
| 2-8 | 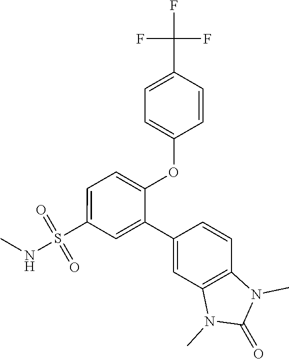 | 3-(1,3-dimethyl-2-oxo-2,3-dihydro- 1H-benzimidazol-5-yl)-N-methyl-4- [4-(trifluoromethyl)phenoxy]benzene- 1-sulfonamide |
| 2-9 | 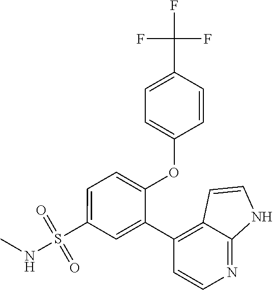 [SMILES: CNS(=O)(=O)c1ccc(Oc2ccc(C(F)(F)F)cc2)c(-c2ccnc3[nH]ccc23)c1] | N-methyl-3-(1H-pyrrolo[2,3-b] pyridin-4-yl)-4-[4- (trifluoromethyl)phenoxy]benzene-1- sulfonamide |
| 2-10 | 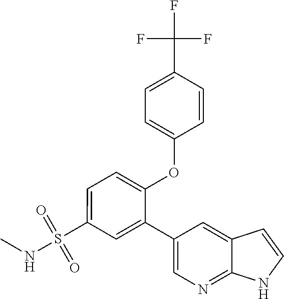 [SMILES: CNS(=O)(=O)c1ccc(Oc2ccc(C(F)(F)F)cc2)c(-c2cnc3[nH]ccc3c2)c1] | N-methyl-3-(1H-pyrrolo[2,3-b] pyridin-5-yl)-4-[4- (trifluoromethyl)phenoxy]benzene- 1-sulfonamide |
| 2-11 | 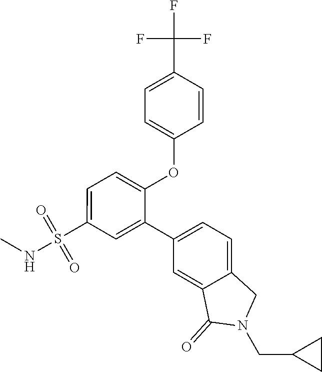 [SMILES: CNS(=O)(=O)c1ccc(Oc2ccc(C(F)(F)F)cc2)c(-c2ccc3c(c2)C(=O)N(CC2CC2)C3)c1] | 3-[2-(cyclopropylmethyl)-3-oxo- 2,3-dihydro-1H-isoindol-5-yl]-N- methyl-4- [4-(trifluoromethyl)phenoxy]benzene- 1-sulfonamide |
| 2-12 | 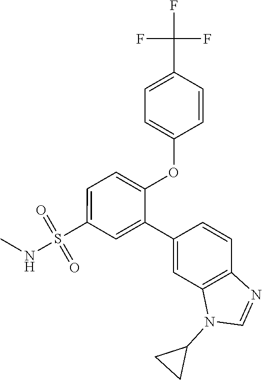 [SMILES: CNS(=O)(=O)c1ccc(Oc2ccc(C(F)(F)F)cc2)c(-c2ccc3ncn(C4CC4)c3c2)c1] | 3-(1-cyclopropyl-1H-benzimidazol-6-yl)- N-methyl-4-[4- (trifluoromethyl)phenoxy]benzene- 1-sulfonamide |
| 2-13 | 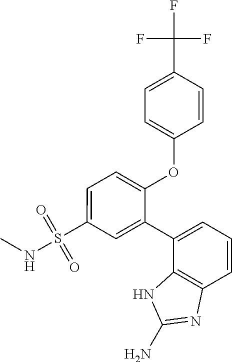 [SMILES: CNS(=O)(=O)c1ccc(Oc2ccc(C(F)(F)F)cc2)c(-c2cccc3nc(N)[nH]c23)c1] | 3-(2-amino-1H-benzimidazol-7-yl)-N- methyl-4-[4- (trifluoromethyl)phenoxy]benzene- 1-sulfonamide |
| 2-14 | 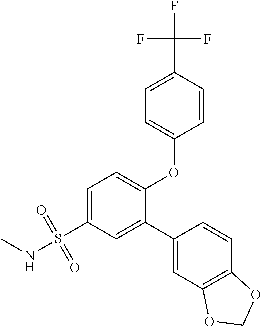 [SMILES: CNS(=O)(=O)c1ccc(Oc2ccc(C(F)(F)F)cc2)c(-c2ccc3c(c2)OCO3)c1] | 3-(2H-1,3-benzodioxol-5-yl)-N- methyl-4-[4- (trifluoromethyl)phenoxy]benzene- 1-sulfonamide |
| 2-15 | 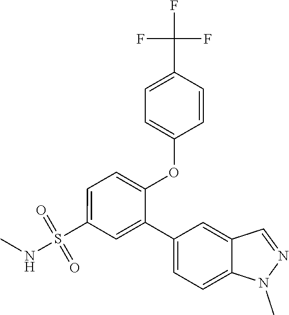 [SMILES: CNS(=O)(=O)c1ccc(Oc2ccc(C(F)(F)F)cc2)c(-c2ccc3c(cnn3C)c2)c1] | N-methyl-3-(1-methyl-1H-indazol- 5-yl)-4-[4- (trifluoromethyl)phenoxy]benzene- 1-sulfonamide |
| 2-16 | 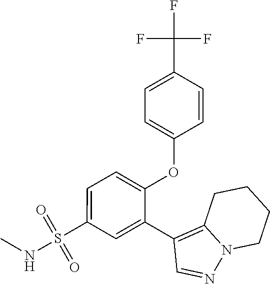 [SMILES: CNS(=O)(=O)c1ccc(Oc2ccc(C(F)(F)F)cc2)c(-c2cnn3c2CCCC3)c1] | N-methyl-3-(4,5,6,7- tetrahydropyrazolo[1,5-a]pyridin-3-yl)- 4-[4-(trifluoromethyl)phenoxy]benzene- 1-sulfonamide |
| 2-17 | 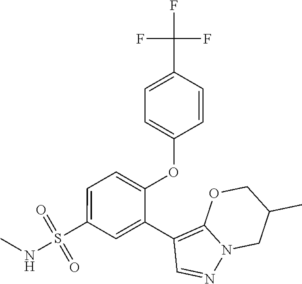 | N-methyl-3-(6-methyl-6,7-dihydro- 5H-pyrazolo[5,1-b][1,3]oxazin-3-yl)- 4-[4-(trifluoromethyl)phenoxy]benzene- 1-sulfonamide |
| 2-18 | 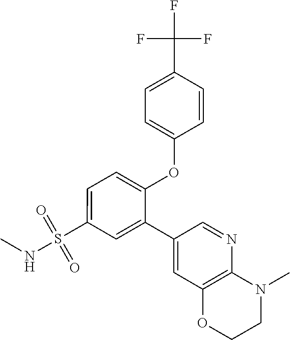 | N-methyl-3-(4-methyl-3,4-dihydro-2H- pyrido[3,2-b][1,4]oxazin-7-yl)-4-[4- (trifluoromethyl)phenoxy]benzene-1- sulfonamide |
| 2-19 | 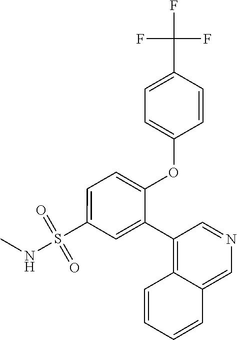 [SMILES: CNS(=O)(=O)c1ccc(Oc2ccc(C(F)(F)F)cc2)c(-c2cncc3ccccc23)c1] | 3-(isoquinolin-4-yl)-N-methyl-4- [4-(trifluoromethyl)phenoxy]benzene- 1-sulfonamide |
| 2-20 | 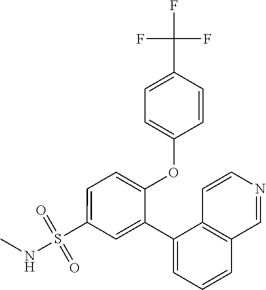 [SMILES: CNS(=O)(=O)c1ccc(Oc2ccc(C(F)(F)F)cc2)c(-c2cccc3cnccc23)c1] | 3-(isoquinolin-5-yl)-N-methyl-4- [4-(trifluoromethyl)phenoxy]benzene- 1-sulfonamide |
| 2-21 | 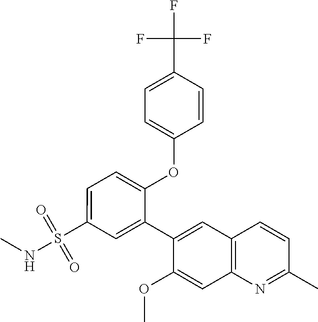 | 3-(7-methoxy-2-methylquinolin-6-yl)- N-methyl-4-[4- (trifluoromethyl)phenoxy]benzene- 1-sulfonamide |
| 2-22 | 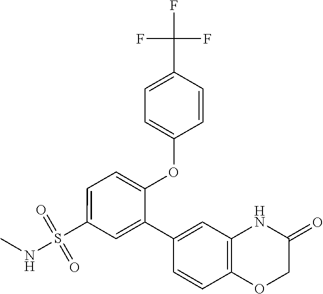 [SMILES: CNS(=O)(=O)c1ccc(Oc2ccc(C(F)(F)F)cc2)c(-c2ccc3c(c2)NC(=O)CO3)c1] | N-methyl-3-(3-oxo-3,4-dihydro-2H- 1,4-benzoxazin-6-yl)-4-[4- (trifluoromethyl)phenoxy]benzene- 1-sulfonamide |
| 2-23 | 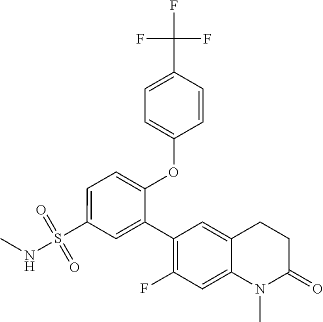 [SMILES: CNS(=O)(=O)c1ccc(Oc2ccc(C(F)(F)F)cc2)c(-c2cc3c(cc2F)N(C)C(=O)CC3)c1] | 3-(7-fluoro-1-methyl-2-oxo-1,2,3,4- tetrahydroquinolin-6-yl)-N-methyl-4- [4-(trifluoromethyl)phenoxy]benzene- 1-sulfonamide |
| 2-24 | 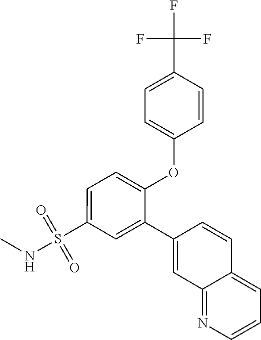 [SMILES: CNS(=O)(=O)c1ccc(Oc2ccc(C(F)(F)F)cc2)c(-c2ccc3cccnc3c2)c1] | N-methyl-3-(quinolin-7-yl)-4- [4-(trifluoromethyl)phenoxy]benzene- 1-sulfonamide |
| 2-25 | 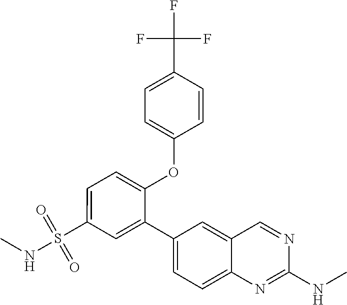 | N-methyl-3- [2-(methylamino)quinazolin- 6-yl]-4-[4- (trifluoromethyl)phenoxy]benzene- 1-sulfonamide |
| 2-26 | 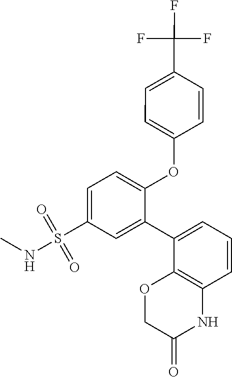 [SMILES: CNS(=O)(=O)c1ccc(Oc2ccc(C(F)(F)F)cc2)c(-c2cccc3c2OCC(=O)N3)c1] | N-methyl-3-(3-oxo-3,4-dihydro- 2H-1,4-benzoxazin-8-yl)-4- [4-(trifluoromethyl)phenoxy]benzene- 1-sulfonamide |
| 2-27 | 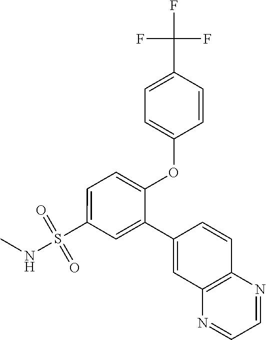 [SMILES: CNS(=O)(=O)c1ccc(Oc2ccc(C(F)(F)F)cc2)c(-c2ccc3nccnc3c2)c1] | N-methyl-3-(quinoxalin-6-yl)-4- [4-(trifluoromethyl)phenoxy]benzene- 1-sulfonamide |
| 2-28 | 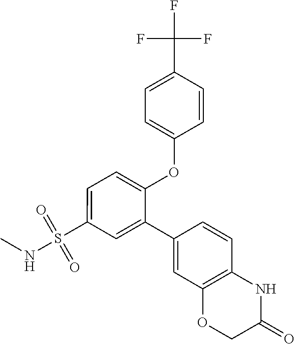 [SMILES: CNS(=O)(=O)c1ccc(Oc2ccc(C(F)(F)F)cc2)c(-c2ccc3c(c2)OCC(=O)N3)c1] | N-methyl-3-(3-oxo-3,4-dihydro- 2H-1,4-benzoxazin-7-yl)-4-[4- (trifluoromethyl)phenoxy]benzene- 1-sulfonamide |
| 2-29 | 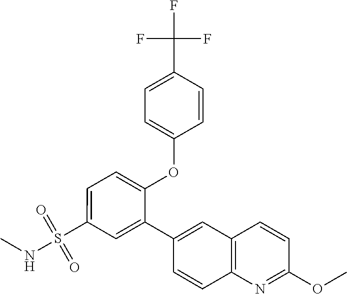 | 3-(2-methoxyquinolin-6-yl)- N-methyl-4-[4- (trifluoromethyl)phenoxy]benzene- 1-sulfonamide |
| 2-30 | 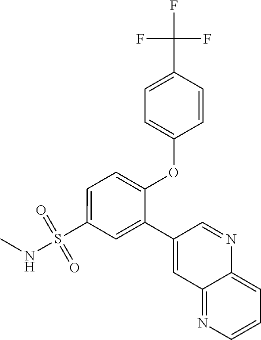 [SMILES: CNS(=O)(=O)c1ccc(Oc2ccc(C(F)(F)F)cc2)c(-c2cnc3cccnc3c2)c1] | N-methyl-3-(1,5-naphthyridin-3-yl)- 4-[4-(trifluoromethyl)phenoxy]benzene- 1-sulfonamide |
| 2-31 | 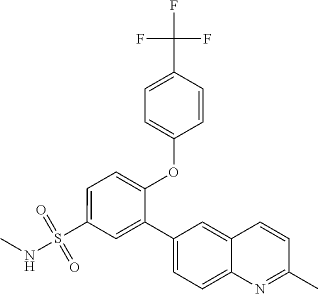 | N-methyl-3-(2-methylquinolin-6-yl)- 4-[4-(trifluoromethyl)phenoxy]benzene- 1-sulfonamide |
| 2-32 | 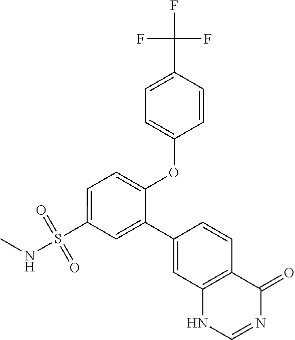 [SMILES: CNS(=O)(=O)c1ccc(Oc2ccc(C(F)(F)F)cc2)c(-c2ccc3c(=O)nc[nH]c3c2)c1] | N-methyl-3-(4-oxo-1,4-dihydroquinazolin- 7-yl)-4-[4- (trifluoromethyl)phenoxy]benzene- 1-sulfonamide |
| 2-33 | 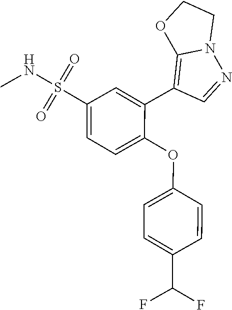 [SMILES: CNS(=O)(=O)c1ccc(Oc2ccc(C(F)F)cc2)c(-c2cnn3c2OCC3)c1] | 4-[4-(difluoromethyl)phenoxy]- 3-(2,3-dihydropyrazolo[5,1-b] [1,3]oxazol-7-yl)-N-methylbenzene- 1-sulfonamide |
| 3-1 | 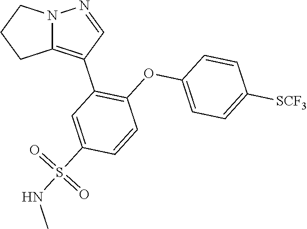 | 3-(5,6-Dihydro-4H-pyrrolo[1,2-b] pyrazol-3-yl)-N-methyl-4-(4- ((trifluoromethyl)thio)phenoxy)benzenesulfonamide |
| 3-2 | 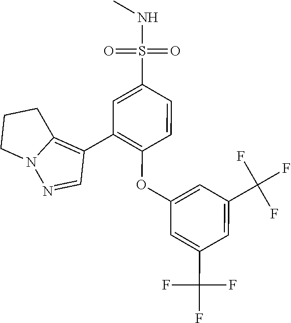 [SMILES: CNS(=O)(=O)c1ccc(Oc2cc(C(F)(F)F)cc(C(F)(F)F)c2)c(-c2cnn3c2CCC3)c1] | 4-[3,5-bis(trifluoromethyl)phenoxy]- 3-(5,6-dihydro-4H-pyrrolo[1,2-b] pyrazol-3-yl)-N-methylbenzene- 1-sulfonamide |
| 3-3 | 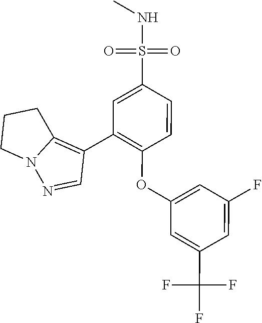 [SMILES: CNS(=O)(=O)c1ccc(Oc2cc(F)cc(C(F)(F)F)c2)c(-c2cnn3c2CCC3)c1] | 3-(5,6-dihydro-4H-pyrrolo[1,2-b] pyrazol-3-yl)-4-[3-fluoro-5- (trifluoromethyl)phenoxy]-N-methylbenzene- 1-sulfonamide |
| 3-4 | 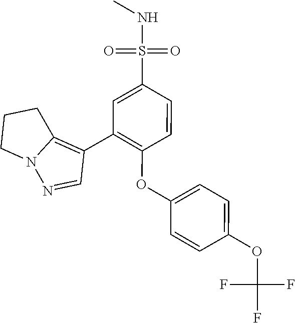 [SMILES: CNS(=O)(=O)c1ccc(Oc2ccc(OC(F)(F)F)cc2)c(-c2cnn3c2CCC3)c1] | 3-(5,6-dihydro-4H-pyrrolo[1,2-b] pyrazol-3-yl)-N-methyl-4-[4- (trifluoromethoxy)phenoxy]benzene- 1-sulfonamide |
| 3-5 | 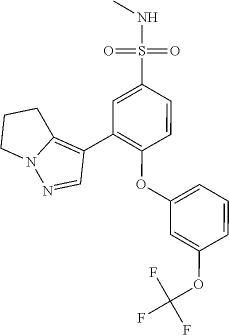 [SMILES: CNS(=O)(=O)c1ccc(Oc2cccc(OC(F)(F)F)c2)c(-c2cnn3c2CCC3)c1] | 3-(5,6-dihydro-4H-pyrrolo[1,2-b] pyrazol-3-yl)-N-methyl-4-[3- (trifluoromethoxy)phenoxy]benzene- 1-sulfonamide |
| 3-6 | 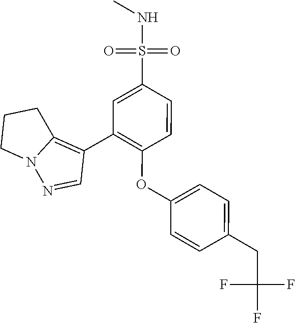 [SMILES: CNS(=O)(=O)c1ccc(Oc2ccc(CC(F)(F)F)cc2)c(-c2cnn3c2CCC3)c1] | 3-(5,6-dihydro-4H-pyrrolo[1,2-b] pyrazol-3-yl)-N-methyl-4-[4- (2,2,2-trifluoroethyl)phenoxy]benzene- 1-sulfonamide |
| 4-1 | 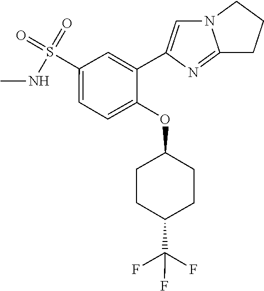 [SMILES: CNS(=O)(=O)c1ccc(O[C@H]2CC[C@H](C(F)(F)F)CC2)c(-c2cn3c(n2)CCC3)c1] | 3-(6,7-Dihydro-5H-pyrrolo[1,2-a] imidazol-2-yl)-N-methyl-4-(((1r,4r)- 4-(trifluoromethyl)cyclohexyl)oxy)benzenesulfonamide |
| 4-2 | 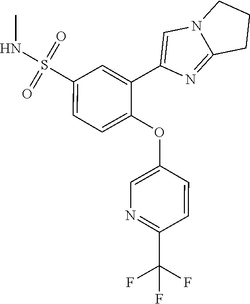 [SMILES: CNS(=O)(=O)c1ccc(Oc2ccc(C(F)(F)F)nc2)c(-c2cn3c(n2)CCC3)c1] | 3-(6,7-dihydro-5H-pyrrolo[1,2-a] imidazol-2-yl)-N-methyl-4- ((6-(trifluoromethyl)pyridin-3- yl)oxy)benzenesulfonamide |
| 4-3 | 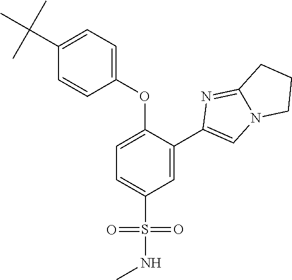 [SMILES: CNS(=O)(=O)c1ccc(Oc2ccc(C(C)(C)C)cc2)c(-c2cn3c(n2)CCC3)c1] | 4-(4-tert-butylphenoxy)-3- (6,7-dihydro-5H-pyrrolo[1,2-a] imidazol-2-yl)-N-methylbenzene- 1-sulfonamide |
| 4-4 | 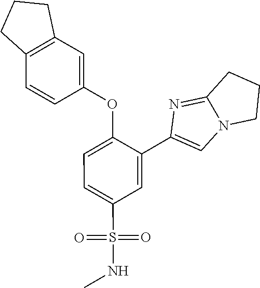 [SMILES: CNS(=O)(=O)c1ccc(Oc2ccc3c(c2)CCC3)c(-c2cn3c(n2)CCC3)c1] | 4-[(2,3-dihydro-1H-inden-5-yl)oxy]- 3-(6,7-dihydro-5H-pyrrolo[1,2-a]imidazol- 2-yl)-N-methylbenzene-1-sulfonamide |
| 4-5 | 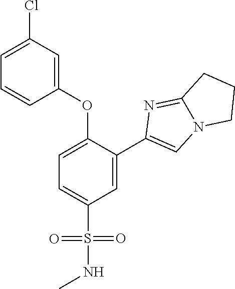 [SMILES: CNS(=O)(=O)c1ccc(Oc2cccc(Cl)c2)c(-c2cn3c(n2)CCC3)c1] | 4-(3-chlorophenoxy)-3- (6,7-dihydro-5H-pyrrolo[1,2-a] imidazol-2-yl)-N-methylbenzene- 1-sulfonamide |
| 4-6 | 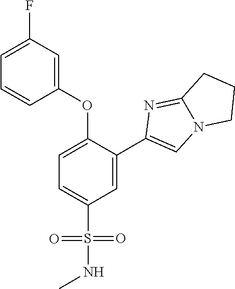 [SMILES: CNS(=O)(=O)c1ccc(Oc2cccc(F)c2)c(-c2cn3c(n2)CCC3)c1] | 3-(6,7-dihydro-5H-pyrrolo [1,2-a]imidazol-2-yl)-4- (3-fluorophenoxy)-N- methylbenzene-1-sulfonamide |
| 4-7 | 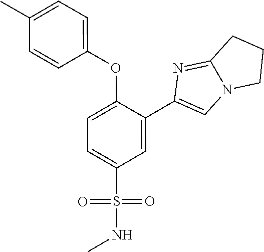 [SMILES: CNS(=O)(=O)c1ccc(Oc2ccc(C)cc2)c(-c2cn3c(n2)CCC3)c1] | 3-(6,7-dihydro-5H-pyrrolo [1,2-a]imidazol-2-yl)-N- methyl-4- (4-methylphenoxy)benzene- 1-sulfonamide |
| 4-8 | 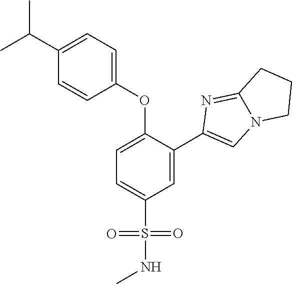 [SMILES: CNS(=O)(=O)c1ccc(Oc2ccc(C(C)C)cc2)c(-c2cn3c(n2)CCC3)c1] | 3-(6,7-dihydro-5H-pyrrolo [1,2-a]imidazol-2-yl)-N- methyl-4-[4- (propan-2-yl)phenoxy]benzene- 1-sulfonamide |
| 4-9 | 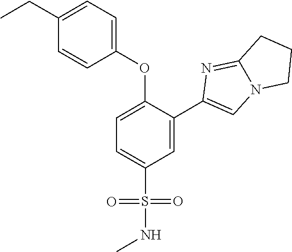 [SMILES: CCc1ccc(Oc2ccc(S(=O)(=O)NC)cc2-c2cn3c(n2)CCC3)cc1] | 3-(6,7-dihydro-5H-pyrrolo [1,2-a]imidazol-2-yl)-4- (4-ethylphenoxy)-N-methylbenzene- 1-sulfonamide |
| 4-10 | 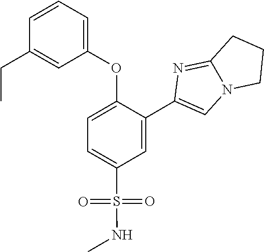 [SMILES: CCc1cccc(Oc2ccc(S(=O)(=O)NC)cc2-c2cn3c(n2)CCC3)c1] | 3-(6,7-dihydro-5H-pyrrolo [1,2-a]imidazol-2-yl)-4- (3-ethylphenoxy)-N- methylbenzene-1-sulfonamide |
| 4-11 | 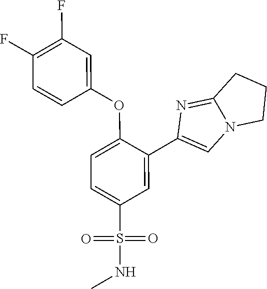 [SMILES: CNS(=O)(=O)c1ccc(Oc2ccc(F)c(F)c2)c(-c2cn3c(n2)CCC3)c1] | 4-(3,4-difluorophenoxy)-3- (6,7-dihydro-5H-pyrrolo[1,2-a] imidazol-2-yl)-N-methylbenzene- 1-sulfonamide |
| 4-12 | 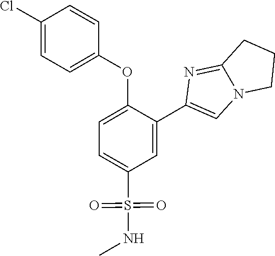 [SMILES: CNS(=O)(=O)c1ccc(Oc2ccc(Cl)cc2)c(-c2cn3c(n2)CCC3)c1] | 4-(4-chlorophenoxy)-3- (6,7-dihydro-5H-pyrrolo[1,2-a] imidazol-2-yl)-N- methylbenzene-1-sulfonamide |
| 4-13 | 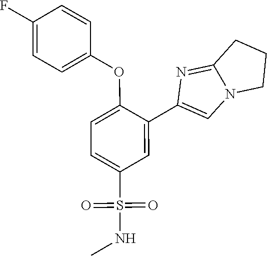 [SMILES: CNS(=O)(=O)c1ccc(Oc2ccc(F)cc2)c(-c2cn3c(n2)CCC3)c1] | 3-(6,7-dihydro-5H-pyrrolo [1,2-a]imidazol-2-yl)-4- (4-fluorophenoxy)-N- methylbenzene-1-sulfonamide |
| 4-14 | 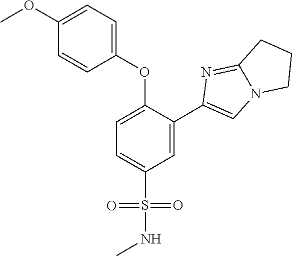 [SMILES: CNS(=O)(=O)c1ccc(Oc2ccc(OC)cc2)c(-c2cn3c(n2)CCC3)c1] | 3-(6,7-dihydro-5H-pyrrolo[1,2-a] imidazol-2-yl)-4- (4-methoxyphenoxy)-N-methylbenzene- 1-sulfonamide |
| 4-15 | 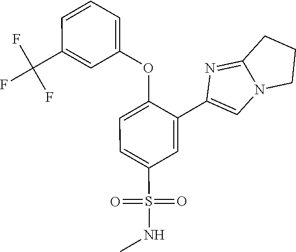 [SMILES: CNS(=O)(=O)c1ccc(Oc2cccc(C(F)(F)F)c2)c(-c2cn3c(n2)CCC3)c1] | 3-(6,7-dihydro-5H-pyrrolo[1,2-a] imidazol-2-yl)-N-methyl-4- [3-(trifluoromethyl)phenoxy]benzene- 1-sulfonamide |
| 4-16 | 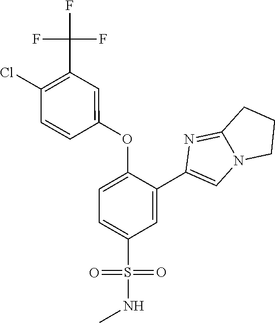 [SMILES: CNS(=O)(=O)c1ccc(Oc2ccc(Cl)c(C(F)(F)F)c2)c(-c2cn3c(n2)CCC3)c1] | 4-[4-chloro-3- (trifluoromethyl)phenoxy]-3- (6,7-dihydro-5H-pyrrolo[1,2-a] imidazol-2-yl)-N-methylbenzene- 1-sulfonamide |
| 4-17 | 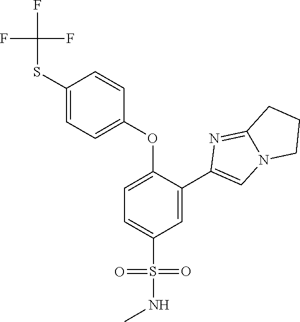 [SMILES: CNS(=O)(=O)c1ccc(Oc2ccc(SC(F)(F)F)cc2)c(-c2cn3c(n2)CCC3)c1] | 3-(6,7-dihydro-5H-pyrrolo[1,2-a] imidazol-2-yl)-N-methyl-4- {4- [(trifluoromethyl)sulfanyl]phenoxy}benzene- 1-sulfonamide |
| 4-18 | 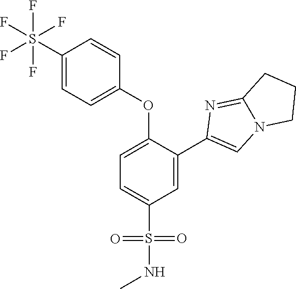 [SMILES: CNS(=O)(=O)c1ccc(Oc2ccc(S(F)(F)(F)(F)F)cc2)c(-c2cn3c(n2)CCC3)c1] | 3-(6,7-dihydro-5H-pyrrolo[1,2-a] imidazol-2-yl)-N-methyl- 4-[4-(pentafluoro- lambda~6~-sulfanyl)phenoxy]benzene- 1-sulfonamide |
| 4-19 | 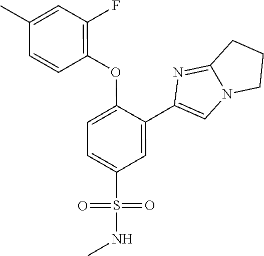 [SMILES: CNS(=O)(=O)c1ccc(Oc2ccc(C)cc2F)c(-c2cn3c(n2)CCC3)c1] | 3-(6,7-dihydro-5H-pyrrolo[1,2-a] imidazol-2-yl)-4-(2-fluoro- 4-methylphenoxy)-N-methylbenzene- 1-sulfonamide |
| 4-20 | 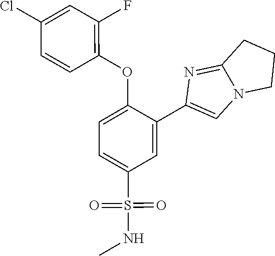 [SMILES: CNS(=O)(=O)c1ccc(Oc2ccc(Cl)cc2F)c(-c2cn3c(n2)CCC3)c1] | 4-(4-chloro-2-fluorophenoxy)- 3-(6,7-dihydro-5H-pyrrolo[1,2-a] imidazol-2-yl)-N-methylbenzene- 1-sulfonamide |
| 4-21 | 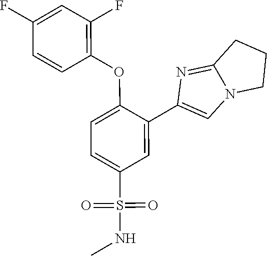 [SMILES: CNS(=O)(=O)c1ccc(Oc2ccc(F)cc2F)c(-c2cn3c(n2)CCC3)c1] | 4-(2,4-difluorophenoxy)-3- (6,7-dihydro-5H-pyrrolo[1,2-a] imidazol-2-yl)-N-methylbenzene- 1-sulfonamide |
| 4-22 | 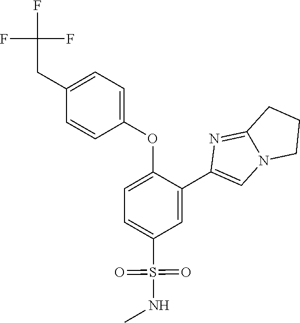 [SMILES: CNS(=O)(=O)c1ccc(Oc2ccc(CC(F)(F)F)cc2)c(-c2cn3c(n2)CCC3)c1] | 4-(2,4-difluorophenoxy)-3- (6,7-dihydro-5H-pyrrolo[1,2-a] imidazol-2-yl)- N-methylbenzene-1-sulfonamide |
| 5-1 | 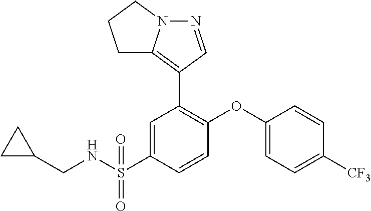 [SMILES: O=S(=O)(NCC1CC1)c1ccc(Oc2ccc(C(F)(F)F)cc2)c(-c2cnn3c2CCC3)c1] | N-(Cyclopropylmethyl)-3- (5,6-dihydro-4H-pyrrolo[1,2-b] pyrazol-3-yl)-4-(4- (trifluoromethyl)phenoxy)benzenesulfonamide |
| 6-1 | 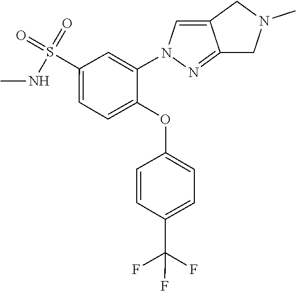 | N-Methyl-3-(5-methyl-5,6-dihydropyrrolo [3,4-c]pyrazol-2(4H)-yl)-4- (4-(trifluoromethyl)phenoxy)benzenesulfonamide |
| 6-2 | 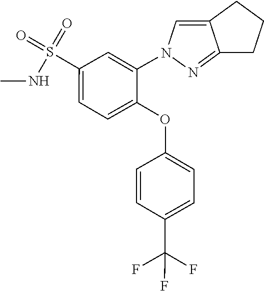 [SMILES: CNS(=O)(=O)c1ccc(Oc2ccc(C(F)(F)F)cc2)c(-n2cc3c(n2)CCC3)c1] | 3-(5,6-dihydrocyclopenta[c]pyrazol- 2(4H)-yl)-N-methyl-4-[4- (trifluoromethyl)phenoxy]benzene- 1-sulfonamide |
| 7-1 | 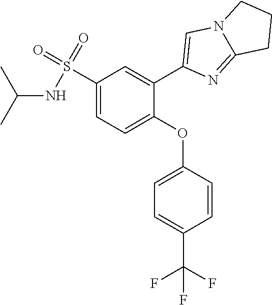 [SMILES: CC(C)NS(=O)(=O)c1ccc(Oc2ccc(C(F)(F)F)cc2)c(-c2cn3c(n2)CCC3)c1] | 3-(6,7-dihydro-5H-pyrrolo[1,2-a] imidazol-2-yl)-N-(propan-2-yl)- 4-[4-(trifluoromethyl)phenoxy]benzene- 1-sulfonamide |
| 7-2 | 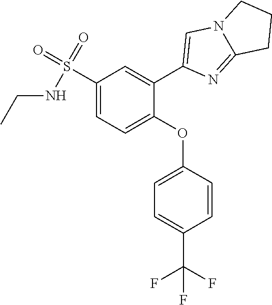 [SMILES: CCNS(=O)(=O)c1ccc(Oc2ccc(C(F)(F)F)cc2)c(-c2cn3c(n2)CCC3)c1] | 3-(6,7-dihydro-5H-pyrrolo[1,2-a] imidazol-2-yl)-N-ethyl-4-[4- (trifluoromethyl)phenoxy]benzene- 1-sulfonamide |
| 7-3 | 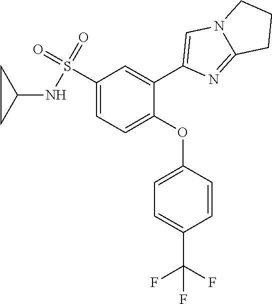 [SMILES: O=S(=O)(NC1CC1)c1ccc(Oc2ccc(C(F)(F)F)cc2)c(-c2cn3c(n2)CCC3)c1] | N-cyclopropyl-3-(6,7-dihydro- 5H-pyrrolo[1,2-a]imidazol-2-yl)- 4-(4- (trifluoromethyl)phenoxy)benzenesulfonamide |
| 8-1 | 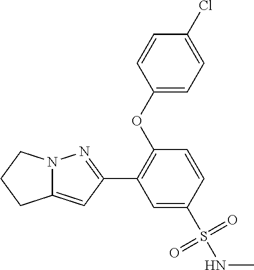 [SMILES: CNS(=O)(=O)c1ccc(Oc2ccc(Cl)cc2)c(-c2cc3n(n2)CCC3)c1] | 4-(4-Chlorophenoxy)-3-(5,6-dihydro- 4H-pyrrolo[1,2-b]pyrazol- 2-yl)-N-methylbenzene-1-sulfonamide |
| 8-2 | 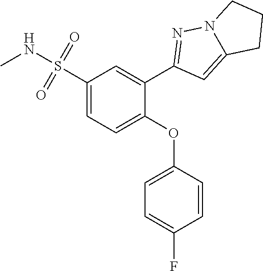 [SMILES: CNS(=O)(=O)c1ccc(Oc2ccc(F)cc2)c(-c2cc3n(n2)CCC3)c1] | 3-(5,6-dihydro-4H-pyrrolo[1,2-b] pyrazol-2-yl)-4-(4-fluorophenoxy)- N-methylbenzene-1-sulfonamide |
| 8-3 | 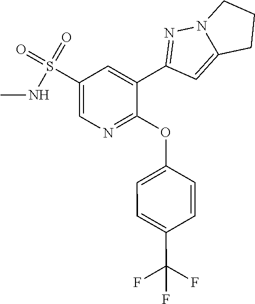 [SMILES: CNS(=O)(=O)c1cnc(Oc2ccc(C(F)(F)F)cc2)c(-c2cc3n(n2)CCC3)c1] | 5-(5,6-dihydro-4H-pyrrolo[1,2-b] pyrazol-2-yl)-N-methyl-6- [4-(trifluoromethyl)phenoxy]pyridine- 3-sulfonamide |
| 8-4 | 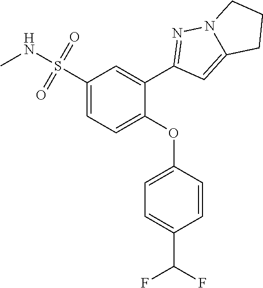 [SMILES: CNS(=O)(=O)c1ccc(Oc2ccc(C(F)F)cc2)c(-c2cc3n(n2)CCC3)c1] | 4-[4-(difluoromethyl)phenoxy]-3- (5,6-dihydro-4H-pyrrolo[1,2-b]pyrazol- 2-yl)-N-methylbenzene-1-sulfonamide |
| 9-1 | 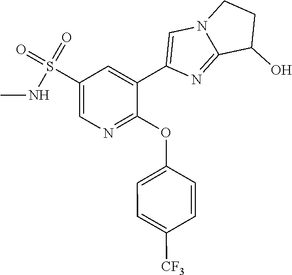 [SMILES: CNS(=O)(=O)c1cnc(Oc2ccc(C(F)(F)F)cc2)c(-c2cn3c(n2)C(O)CC3)c1] | 3-(7-Hydroxy-6,7-dihydro- 5H-pyrrolo[1,2-a]imidazol-2-yl)- N-methyl-4-(4- (trifluoromethyl)phenoxy)benzenesulfonamide |
| 9-2 | 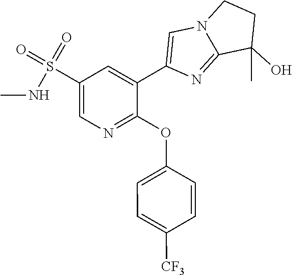 [SMILES: CNS(=O)(=O)c1cnc(Oc2ccc(C(F)(F)F)cc2)c(-c2cn3c(n2)C(C)(O)CC3)c1] | 3-(7-Hydroxy-7-methyl-6,7-dihydro- 5H-pyrrolo[1,2-a]imidazol-2-yl)- N-methyl-4-(4- (trifluoromethyl)phenoxy)benzenesulfonamide |
| 10-1 | 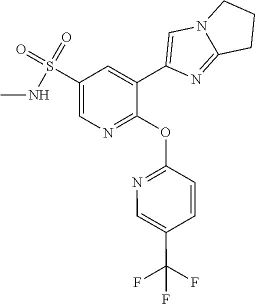 [SMILES: CNS(=O)(=O)c1cnc(Oc2ccc(C(F)(F)F)cn2)c(-c2cn3c(n2)CCC3)c1] | 3-(6,7-Dihydro-5H-pyrrolo[1,2-a] imidazol-2-yl)-N-methyl-4-((5- (trifluoromethyl)pyridin- 2-yl)oxy)benzenesulfonamide |
| 11-1 | 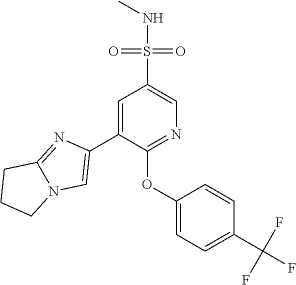 [SMILES: CNS(=O)(=O)c1cnc(Oc2ccc(C(F)(F)F)cc2)c(-c2cn3c(n2)CCC3)c1] | 5-(6,7-dihydro-5H-pyrrolo[1,2-a] imidazol-2-yl)-N-methyl-6-[4- (trifluoromethyl)phenoxy]pyridine- 3-sulfonamide |

or a pharmaceutically acceptable salt thereof.

An embodiment of the invention comprises a compound selected from Ex. No. 1-1, 1-2, 1-3, 2-1, 4-1, 8-3, 10-1 and 11-1 or a pharmaceutically acceptable salt thereof. A further embodiment of the invention comprises a compound selected from Ex. No. 1-1, 1-2, 1-3, 2-1, 4-1, and 11-1 or a pharmaceutically acceptable salt thereof.

In an embodiment, the invention provides a compound of Formula I or IA, where Ring A is selected from

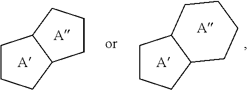 [SMILES: C1CC2CCCC2C1 ; C1CCC2CCCC2C1]

wherein the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom.

In another embodiment, the invention provides a compound of Formula I or IA, where Ring A is selected from

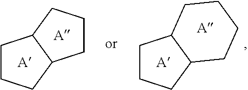 [SMILES: C1CC2CCCC2C1 ; C1CCC2CCCC2C1]

wherein the A′ ring may contain one, two or three heteroatoms selected from N, O or S atoms and the ring may be aromatic or aliphatic and the A″ ring may contain one, two or three heteroatoms selected from N, O or S atoms and the ring may be aromatic or aliphatic and at least one of A′ or A″ contains at least one heteroatom.

In another embodiment of Formula I or Formula IA, Ring A is selected from dihydropyrroloimidazolyl, dihydroimidazolyloxazinyl, dihydropyrazolooxazolyl, dihydropyrrolopyrazolyl, imidazopyridinyl, imidazopyrazinyl, imidazothiazolyl, indazolyl, tetrahydroimidazopyridinyl, triazolopyrimidinyl, tetrahydrothienopyridinyl, tetrahydroimidazopyrazinyl, pyrazolopyrimidinyl, thiazolopyridinyl, benzimidazolyl, benzothiazolyl, dihydropyrrolothiazolyl, triazolopyrazinyl or dihydroimidazopyrazolodiazepinyl. In another embodiment, Ring A is selected from dihydropyrroloimidazolyl, dihydroimidazolyloxazinyl, dihydropyrazolooxazolyl, or dihydropyrrolopyrazolyl. In another embodiment, Ring A is selected from dihydropyrroloimidazolyl or dihydropyrazolooxazolyl. In another embodiment, Ring A is dihydropyrroloimidazolyl.

In an embodiment, the invention provides a compound of Formula I or IA, wherein Ring B is selected from phenyl, cyclohexyl or pyridinyl. In another embodiment of Formula I or IA, Ring B is selected from phenyl or pyridinyl. In another embodiment of Formula I or IA, Ring B is phenyl. In another embodiment of Formula I or IA, Ring B is pyridinyl.

In an embodiment, the invention provides a compound of Formula I or IA, wherein Ring D is phenyl. In another embodiment of Formula I or IA, Ring D is pyridinyl.

In an embodiment, the invention provides a compound of Formula I or IA, wherein R¹ is selected from C₃-C₁₀ cycloalkyl or C₁₋₆alkyl, wherein said alkyl is optionally substituted with one to three groups selected from C₁₋₆alkyl, C₃-C₁₀ cycloalkyl, CF₃, or (CR₂)_{z}OR. In another embodiment, the invention provides a compound of Formula I or IA, wherein R is C₁₋₆alkyl, wherein said alkyl is optionally substituted with one to three groups selected from C₁₋₆alkyl, C₃-C₁₀ cycloalkyl, CF₃, or (CR₂)_{z}OR. In another embodiment, the invention provides a compound of Formula I or IA, wherein R¹ is selected from CH₃.

In an embodiment, the invention provides a compound of Formula I or IA, wherein R² is independently selected from OR, oxo, halo, or —C₁₋₆alkyl, where said alkyl is optionally substituted with one to three groups selected from —C₁₋₆alkyl, C₃-C₁₀ cycloalkyl, —C(O)OR, OR or halo.

In an embodiment, the invention provides a compound of Formula I or IA, wherein R³ is selected from —C₁₋₆alkyl, CF₃, CHF₂, halo, or OR, where said alkyl is optionally substituted with one to three groups from —C₁₋₆alkyl, OR, CF₃, or halo. In another embodiment, the invention provides a compound of Formula I or IA, wherein R³ is selected from —C₁₋₆alkyl, CF₃, CHF₂, or halo, where said alkyl is optionally substituted with one to three groups from —C₁₋₆ alkyl, CF₃, or halo. In another embodiment, the invention provides a compound of Formula I or IA, wherein R³ is CF₃.

In an embodiment of Formula I or IA, variable n is selected from 0, 1, 2 or 3. In an embodiment of Formula I or IA, variable p is selected from 1 or 2.

In one embodiment the present invention provides pharmaceutical compositions comprising a compound of the invention, for example, a compound of Formula I or a pharmaceutically acceptable salt thereof, and at least one pharmaceutical excipient.

In one embodiment, the present invention provides a method for treating cancer in a patient, comprising administering to the patient a compound of Formula I or Formula IA, or a pharmaceutically acceptable salt thereof, or a pharmaceutical composition comprising a compound of Formula I or Formula IA. In a further embodiment, the cancer is associated with increased YAP1 and/or TAZ expression.

In one embodiment, the present invention provides a method for inhibiting the progress of cancer in a patient, comprising administering to the patient a compound of Formula I or Formula IA, or a pharmaceutically acceptable salt thereof, or a pharmaceutical composition comprising a compound of Formula I or Formula IA.

In one embodiment, the present invention provides a method of treating a disease or disorder in which Hippo pathway inhibition is beneficial, comprising administering to a patient in need thereof a therapeutically effective amount of a compound of Formula I or Formula IA, or a pharmaceutically acceptable salt thereof, or a pharmaceutical composition comprising a compound of Formula I or Formula IA. In a further embodiment, the disease or disorder is a cellular proliferative disorder. In a further embodiment, the cellular proliferative disorder is cancer.

In one embodiment, the present invention provides for the use of a compound of Formula I or Formula IA, or a pharmaceutically acceptable salt thereof, or a pharmaceutical composition containing a compound of Formula I or Formula IA, for treating cancer in a patient. In one embodiment, the present invention provides for the use of a compound of Formula I or Formula IA, or a pharmaceutically acceptable salt thereof, or a pharmaceutical composition containing a compound of Formula I or Formula IA, for the preparation of a medicament useful for the prevention of a cell proliferative disorder.

In the description that follows conventional structural representation is employed and includes conventional stereochemical notation for certain asymmetric carbon centers. Thus, structural representation of compounds of the invention includes conventional stereochemical notation for some asymmetric carbon centers shown in the example compounds. Accordingly, in such instances, solid black “wedge” bonds represent bonds projecting from the plane of the reproduction medium, “hashed wedge” bonds representing descending bonds into the plane of the reproduction medium, and a “wavey” line appended to a carbon bearing a double bond indicates both possible cis and trans orientations are included. As is conventional, plain solid lines represent all spatial configurations for the depicted bonding. Accordingly, where no specific stereochemical notation is supplied the representation contemplates all stereochemical and spatial orientations of the structural features.

As is shown in the examples of the invention, and mentioned above, particular asymmetric carbon centers are structurally represented using conventional “Solid Wedge” and “Hash Wedge” bonding representation. For the most part, absolute configuration has not been determined for the example compounds, but has been assigned by analogy to specific example compounds of known stereochemical configurations (determined by X-ray crystallography) prepared using the same or analogous reaction conditions and starting reagents and isolated under the same chromatographic conditions. Accordingly, specific assignment of the configurations structurally represented herein is meant to identify the specific compounds prepared has having an excess of one particular stereoisomer and is not put forth herein necessarily as being a statement of the absolute determination of the stereochemical structure of said compound unless otherwise noted in the data presented.

In some embodiments, the compounds disclosed herein contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH.

It will be appreciated that where isomeric mixtures are obtained, the preparation of individual stereoisomers in significant percentages of enantiomeric excess can be carried out, if desired, by separation of the mixture using customary methods, for example by chromatography or crystallization, or by the use of stereochemically uniform starting materials for the synthesis described, or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product.

Where indicated herein, absolute stereochemistry is determined by X-ray crystallography of crystalline products or crystalline intermediates which are derivatized, if necessary, with a reagent containing a stereogenic center of known configuration. Unless a particular isomer, salt, solvate (including hydrates) or solvated salt of such racemate, enantiomer, or diastereomer is indicated, the present invention includes all such isomers, as well as salts, solvates (including hydrates) and solvated salts of such racemates, enantiomers, diastereomers and mixtures thereof.

Where a wavey line terminates a conventional bond (as opposed to connecting two atoms within a structure) it indicates a point of bonding to a structure, e.g.:

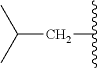 [SMILES: *CC(C)C]

indicates a the secondary-butyl moiety is bonded via the methylene group via the bond terminated with the wavey line. Where an alphabetical notation is used to depict a substituent moiety, a dash is employed to indicate the point of bonding to the indicated substrate, e.g.: —CH₂—C(O)—CH₂C₁ indicates the acetyl chloride moiety is bonded via the methylene portion of the moiety.

Where compounds of Formula I are capable of tautomerization, all individual tautomers as well as mixtures thereof are included in the scope of this invention.

When any variable (e.g., R, R¹, n, alkyl, etc.) occurs more than one time in any constituent or in Formula I, its definition on each occurrence is independent of its definition at every other occurrence unless otherwise specified at the point of definition. One of ordinary skill in the art will recognize that choice of combinations of the various substituents defined in a structural representation, i.e. R¹, R², etc., are to be chosen in conformity with well-known principles of chemical structure connectivity and stability, and combinations of substituents and/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formula I.

Where any variable or moiety is expressed in the form of a range, e.g. (—CH₂—)₁₋₄, both of the extrema of the specified range are included (i.e. 1 and 4 in the example) as well as all of the whole number values in between (i.e. 2 and 3 in the example).

It is understood that reference to “Formula I” also encompasses compounds of Formula IA, unless indicated otherwise.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, in some embodiments, ranges and amounts are expressed as “about” a particular value or range. About also includes the exact amount. Hence “about 5 μL” means “about 5 μL” and also “5 μL.” Generally, the term “about” includes an amount that is expected to be within experimental error.

“Optional” or “optionally” means that a subsequently described event or circumstance may or may not occur and that the description includes instances when the event or circumstance occurs and instances in which it does not. For example, “optionally substituted alkyl” means that the alkyl radical may or may not be substituted and that the description includes both substituted alkyl radicals and alkyl radicals having no substitution.

The term “optionally substituted” or “substituted” means that the referenced group is optionally substituted with one or more additional group(s).

“Alkyl” refers to a straight or branched hydrocarbon chain radical consisting solely of carbon and hydrogen atoms, containing no unsaturation. Alkyl may contain one to fifteen carbon atoms (e.g., C₁-C₁₅ alkyl), unless otherwise stated. In certain embodiments, an alkyl comprises one to eight carbon atoms (e.g., C₁-C₈ alkyl). In other embodiments, an alkyl comprises one to six carbon atoms (e.g., C₁-C₆ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, the alkyl group is selected from methyl, ethyl, 1-propyl (n-propyl), 1-methylethyl (iso-propyl), I-butyl (n-butyl), 1-methylpropyl (sec-butyl), 2-methylpropyl (iso-butyl), 1,1-dimethylethyl (tert-butyl), 1-pentyl (n-pentyl). In other embodiments, the alkyl group is methyl. The alkyl is attached to the rest of the molecule by a single bond.

“Halogen” or “halo” as used herein means fluoro, chloro, bromo and iodo.

As used herein, “cycloalkyl” is intended to include cyclic saturated aliphatic hydrocarbon groups having the specified number of carbon atoms. Preferably, cycloalkyl is C₃-C₁₀ cycloalkyl. Examples of such cycloalkyl elements include, but are not limited to, cyclopropyl, cyclobutyl, cyclopentyl, cyclohexyl and cycloheptyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl. In an embodiment of the instant invention, aryl is phenyl or naphthyl. In a further embodiment, aryl is phenyl.

The term heterocyclyl, heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic can include heteroaryl moieties when two rings are fused together. Examples of heterocyclic elements include, but are not limited to, azabicyclo[2.2.1]heptanyl, azepanyl, azetidinyl, benzodioxolyl, chromanyl, dihydrobenzofuryl, dihydrobenzothienyl, dihydrobenzothiopyranyl, dihydrobenzothiopyranyl sulfone, dihydro-pyrrolo[1,2-b]pyrazolyl, dihydro-5H-pyrrolo[1,2-α]imidazol-2-yl 1,3-dioxolanyl, imidazolidinyl, indolinyl, isochromanyl, isoindolinyl, morpholinyl, oxa-5-azabicyclo[2.2.1]heptanyl, 2-oxopiperazinyl, 2-oxopiperdinyl, 2-oxopyrrolidinyl, piperidyl, piperazinyl, pyrazolidinyl, pyrrolidinyl, tetrahydrofuryl, tetrahydroisoquinolinyl, tetrahydroquinolinyl, and thiamorpholinyl.

“Heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heteroaryl groups include, but are not limited to, azepinyl, furanyl, furyl, imidazolyl, indolinyl, indolyl, isochromanyl, isoindolinyl, isoxazolyl, isoquinolinyl, isothiazolyl, naphthyridinyl, oxazolyl, oxadiazolyl, pyridyl, pyrazinyl, pyrazolyl, pyridazinyl, pyrimidinyl, 5H-pyrrolo[2,3-b]pyrazinyl, pyrrolyl, quinazolinyl, quinolinyl, tetrahydroisoquinolinyl, tetrahydroquinolinyl, tetrazolyl, thiazolyl, thienofuryl, thienothienyl, thienyl, triazolyl and the like. In an embodiment, heteroaryl is selected from furyl, imidazolyl, indolyl, isoxazolyl, oxazolyl, oxadiazolyl, pyridyl, pyrazinyl, pyrazolyl, pyridazinyl, pyrimidinyl, 5H-pyrrolo[2,3-b]pyrazinyl, tetrazolyl, thiazolyl, thienyl, triazolyl and the like.

In an embodiment of the invention, Ring A of Formula I or IA may be represented as:

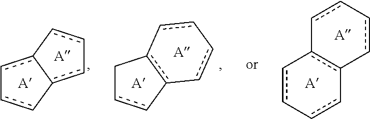

wherein the A′ ring may contain one, two or three heteroatoms selected from N, O or S atoms and the ring may be aromatic or aliphatic (as shown by the dashed lines) and the A″ ring may contain one, two or three heteroatoms selected from N, O or S atoms and the ring may be aromatic or aliphatic (as shown by the dashed lines) and at least one of A′ or A″ contains at least one heteroatom.

In another embodiment of the invention, Ring A of Formula I or IA may be represented as:

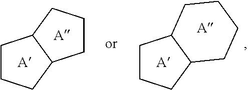 [SMILES: C1CC2CCCC2C1 ; C1CCC2CCCC2C1]

where the A′ ring may contain one, two or three heteroatoms selected from N, O or S atoms and the ring may be aromatic or aliphatic and the A″ ring may contain one, two or three heteroatoms selected from N, O or S atoms and the ring may be aromatic or aliphatic and at least one of A′ or A″ contains at least one heteroatom;

In an embodiment of the instant invention, Ring A of Formula I or Formula IA is selected from 5,6-Dihydro-4H-pyrrolo[1,2-b]pyrazol-2-yl; 5,6-Dihydro-8H-imidazo[2,1-c][1,4]oxazin-2-yl; imidazo[1,2-a]pyridin-3-yl; 5,6-dihydro-4H-pyrrolo[1,2-b]pyrazol-3-yl; 6,7-dihydro-5H-pyrrolo[1,2-a]imidazol-3-yl; imidazo[1,5-a]pyrazin-3-yl; 2H-indazol-3-yl; 5,6,7,8-tetrahydroimidazo[1,2-a]pyridin-2-yl; [1,2,4]triazolo[1,5-a]pyrimidin-2-yl; [1,3]thiazolo[5,4-b]pyridin-2-yl; 1H-benzimidazol-2-yl; 1,2-benzothiazol-3-yl; 5,6-dihydro-4H-pyrrolo[3,4-d][1,3]thiazol-2-yl; [1,2,4]triazolo[1,5-a]pyrazin-2-yl; 4,5,6,7-tetrahydrothieno[3,2-c]pyridin-2-yl; 5,6,7,8-tetrahydroimidazo[1,2-a]pyrazin-2-yl; imidazo[2,1-b][1,3]thiazol-6-yl; pyrazolo[1,5-a]pyrimidin-2-yl; 5,6-dihydro-8H-imidazo[5,1-c][1,4]oxazin-1-yl; imidazo[1,2-b]pyridazin-3-yl; imidazo[1,2-a]pyrimidin-3-yl; imidazo[1,2-a]pyridin-2-yl; 1,3-benzothiazol-2-yl; 1,3-benzoxazol-2-yl; pyrazolo[1,5-a]pyridin-2-yl; 4,5,6,7-tetrahydropyrazolo[1,5-a]pyridin-2-yl; 5,6,7,8-tetrahydroimidazo[1,5-a]pyridin-1-yl; 6,7-dihydro-5H-pyrrolo[1,2-a]imidazol-2-yl; 2,3-dihydroimidazo[2,1-b][1,3]oxazol-6-yl; 2,3-dihydro-TH-imidazo[1,2-b]pyrazol-6-yl; 2,3-dihydropyrazolo[5,1-b]oxazol-7-yl; cinnoline-7-yl; 1H-benzimidazol-7-yl; 2,3-dihydro-TH-indol-5-yl; 2,3-dihydro-TH-benzimidazol-5-yl; 1,3-benzothiazol-5-yl; 1H-indol-7-yl; 2,3-dihydro-1H-benzimidazol-5-yl; 1H-pyrrolo[2,3-b]pyridin-4-yl; 1H-pyrrolo[2,3-b]pyridin-5-yl; 2,3-dihydro-TH-isoindol-5-yl; 1H-benzimidazol-6-yl; 1H-benzimidazol-7-yl; 2H-1,3-benzodioxol-5-yl; 1H-indazol-5-yl; 4,5,6,7-tetrahydropyrazolo[1,5-a]pyridin-3-yl; 6,7-dihydro-5H-pyrazolo[5,1-b][1,3]oxazin-3-yl; isoquinolin-4-yl; isoquinolin-5-yl; quinolin-6-yl; quinolin-7-yl; quinazolin-6-yl; quinoxalin-6-yl; 3,4-dihydro-2H-1,4-benzoxazin-6-yl; 1,2,3,4-tetrahydroquinolin-6-yl; 3,4-dihydro-2H-1,4-benzoxazin-8-yl; 3,4-dihydro-2H-1,4-benzoxazin-7-yl; 1,5-naphthyridin-3-yl; 1,4-dihydroquinazolin-7-yl; 2,3-dihydropyrazolo[5,1-b][1,3]oxazol-7-yl; 5,6-dihydropyrrolo[3,4-c]pyrazol-2(4H)-yl; 5,6-dihydrocyclopenta[c]pyrazol-2(4H)-yl; 5,6-dihydro-4H-pyrrolo[1,2-b]pyrazol-2-yl; 6,7-dihydro-5H-pyrrolo[1,2-a]imidazol-2-yl; or 6-azaspiro[2.4]hept-5-en-5-yl.

For use in medicine, the salts of the compounds of Formula I will be pharmaceutically acceptable salts. Other salts may, however, be useful in the preparation of the compounds according to the invention or of their pharmaceutically acceptable salts. “Pharmaceutically acceptable salt” includes both acid and base addition salts. A pharmaceutically acceptable salt of any one of the compounds described herein is intended to encompass any and all pharmaceutically suitable salt forms. Pharmaceutically acceptable salts of the compounds described herein are optionally pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety).

In some embodiments, acid addition salts of basic compounds are prepared by contacting the free base forms with a sufficient amount of the desired acid to produce the salt according to methods and techniques with which a skilled artisan is familiar.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,” J. Pharm. Sci., 1977:66:1-19.

If the compounds of Formula I simultaneously contain acidic and basic groups in the molecule the invention also includes zwitterions, in addition to the salt forms described above.

### Pharmaceutical Compositions

In certain embodiments, the compound as described herein is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, PA (2005)), the disclosure of which is hereby incorporated herein by reference in its entirety.

This invention further relates to a pharmaceutical composition comprising an effective amount of at least one compound of Formula I and a pharmaceutically acceptable carrier. The composition may comprise, but is not limited to, one or more buffering agents, wetting agents, emulsifiers, suspending agents, lubricants, adsorbents, surfactants, preservatives and the like. The composition may be formulated as a solid, liquid, gel or suspension for oral administration (e.g., drench, bolus, tablet, powder, capsule, mouth spray, emulsion); parenteral administration (e.g., subcutaneous, intramuscular, intravenous, epidural injection); topical application (e.g., cream, ointment, controlled-released patch, spray); intravaginal, intrarectal, transdermal, ocular, or nasal administration. In a further embodiment, the pharmaceutical composition of the present invention may be formulated for parenteral administration, such as an intravenous formulation.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of Formula I, is included in an amount sufficient to produce the desired effect upon the process or condition of diseases. Accordingly, the pharmaceutical compositions of the present invention encompass any composition made by admixing a compound of the present invention and a pharmaceutically acceptable carrier.

The formulations of this invention include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions are formulated as a unit dose, and/or are formulated for oral or subcutaneous administration.

In some instances, exemplary pharmaceutical compositions are used in the form of a pharmaceutical preparation, for example, in solid, semisolid, or liquid form, which includes one or more of a disclosed compound, as an active ingredient, in admixture with an organic or inorganic carrier or excipient suitable for external, enteral, or parenteral applications. In some embodiments, the active ingredient is compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets in some instances, the principal active ingredient is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a disclosed compound or a non-toxic pharmaceutically acceptable salt thereof. When referring to these preformulation compositions as homogeneous, it is meant that the active ingredient is dispersed evenly throughout the composition so that the composition is readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the compositions also comprise buffering agents in some embodiments. Solid compositions of a similar type are also employed as fillers in soft and hard-filled gelatin capsules using such excipients as lactose or milk sugars, as well as high molecular weight polyethylene glycols and the like.

In some instances, a tablet is made by compression or molding, optionally with one or more accessory ingredients. Compressed tablets are prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets are made by molding in a suitable machine a mixture of the subject composition moistened with an inert liquid diluent. Tablets, and other solid dosage forms, such as dragees, capsules, pills and granules, are optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms contain optionally inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, optionally contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In some embodiments, formulations for rectal or vaginal administration are presented as a suppository, which are prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component is optionally mixed under sterile conditions with a pharmaceutically acceptable carrier, and with any preservatives, buffers, or propellants which are required in some embodiments.

In some embodiments, the ointments, pastes, creams and gels contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In some embodiments, powders and sprays contain, in addition to a subject composition, excipients such as lactose, talc, silicic acid, aluminum hydroxide, calcium silicates and polyamide powder, or mixtures of these substances. Sprays additionally contain customary propellants, such as chlorofluorohydrocarbons and volatile unsubstituted hydrocarbons, such as butane and propane.

Compositions and compounds disclosed herein are alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are used because they minimize exposing the agent to shear, which result in degradation of the compounds contained in the subject compositions in some embodiments. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions suitable for parenteral administration comprise a subject composition in combination with one or more pharmaceutically-acceptable sterile isotonic aqueous or non-aqueous solutions, dispersions, suspensions or emulsions, or sterile powders which are reconstituted into sterile injectable solutions or dispersions just prior to use, which optionally contain antioxidants, buffers, bacteriostats, solutes which render the formulation isotonic with the blood of the intended recipient or suspending or thickening agents.

Examples of suitable aqueous and non-aqueous carriers employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. In some embodiments, proper fluidity is maintained, for example, by the use of coating materials, such as lecithin, by the maintenance of the required particle size in the case of dispersions, and by the use of surfactants

In some embodiments, the dose of the composition comprising at least one compound as described herein differ, depending upon the patient's (e.g., human) condition, that is, stage of the disease, general health status, age, and other factors that a person skilled in the medical art will use to determine dose.

In some instances, pharmaceutical compositions are administered in a manner appropriate to the disease to be treated (or prevented) as determined by persons skilled in the medical arts. An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. In some embodiments, the optimal dose depends upon the body mass, weight, or blood volume of the patient.

In some embodiments, oral doses typically range from about 1.0 mg to about 1000 mg, one to four times, or more, per day.

As the term is used herein, “patients” (alternatively “subjects”) refers to an animal, preferably a mammal, and in particular a human, in need of assessment via an imaging study. As used herein, the term “administration” and variants thereof (e.g., “administering” a compound) in reference to a compound of Formula I means providing the compound, or a pharmaceutically acceptable salt thereof, to a subject in need of treatment.

As used herein, “treatment” or “treating” are used interchangeably herein. These terms refers to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is afflicted with the underlying disorder in some embodiments. For prophylactic benefit, in some embodiments, the compositions are administered to a patient at risk of developing a particular disease, or to a patient reporting one or more of the physiological symptoms of a disease, even though a diagnosis of this disease has not been made.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. In some embodiments, examples of isotopes that are incorporated into compounds of the disclosure include isotopes of hydrogen, carbon, nitrogen, oxygen, phosphorous, sulfur, fluorine, and chlorine, such as ²H, ³H, ¹³C, ¹⁴C, ¹⁵N, ¹⁸O, ¹⁷O, ³¹P, ³²p, ³⁵S, ¹⁸F, and ³⁶C₁, respectively. Compounds described herein, and the metabolites, pharmaceutically acceptable salts, esters, prodrugs, solvates, hydrates or derivatives thereof which contain the aforementioned isotopes and/or other isotopes of other atoms are within the scope of this disclosure. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### The Hippo Signaling Network

The Hippo signaling network (also known as the Salvador/Warts/Hippo (SWH) pathway) is a master regulator of cell proliferation, death, and differentiation. In some embodiments, the main function of the Hippo signaling pathway is to regulate negatively the transcriptional co-activators Yes-associated protein (YAP) and its paralogue, the transcriptional co-activator with PDZ-binding motif (TAZ; also known as WWTR1). The Hippo kinase cascade phosphorylates and inhibits YAP/TAZ by promoting its cytoplasmic retention and degradation, thereby inhibiting the growth promoting function regulated under the YAP/TAZ control. In an un-phosphorylated/de-phosphorylated state, YAP, also known as YAP1 or YAP65, together with TAZ, are transported into the nucleus where they interact with TEAD family of transcription factors to upregulate genes that promote proliferation and migration, and inhibit apoptosis. In some instances, unregulated upregulation of these genes involved in proliferation, migration, and anti-apoptosis leads to development of cancer. In some instances, overexpression of YAP/TAZ is associated with cancer.

Additional core members of the Hippo signaling pathway comprise the serine/threonine kinases MST1/2 (homologues of Hippo Hpo in Drosophila), Lats1/2 (homologues of Warts Wts), and their adaptor proteins Sav1 (homologue of Salvador/Sav) and Mob (MOBKL1A and MOBKL1B; homologues of Mats), respectively. In general, MST1/2 kinase complexes with the scaffold protein Sav1, which in turn phosphorylates and activates Lats1/2 kinase. Lats1/2 is also activated by the scaffold protein Mob. The activated Lats1/2 then phosphorylates and inactivates YAP or its paralog TAZ. The phosphorylation of YAP/TAZ leads to their nuclear export, retention within the cytoplasm, and degradation by the ubiquitin proteasome system.

In some instances, Lats1/2 phosphorylates YAP at the [HXRXXS] consensus motifs. YAP comprises five [HXRXXS] consensus motifs, wherein X denotes any amino acid residue. In some instances, Lats1/2 phosphorylates YAP at one or more of the consensus motifs. In some instances, Lats1/2 phosphorylates YAP at all five of the consensus motifs. In some instances, Lats1/2 phosphorylate at the 5127 amino acid position. The phosphorylation of YAP 5127 promotes 14-3-3 protein binding and results in cytoplasmic sequestration of YAP. Mutation of YAP at the S127 position thereby disrupts its interaction with 14-3-3 and subsequently promotes nuclear translocation.

Additional phosphorylation occurs at the S38 1 amino acid position in YAP. Phosphorylation of YAP at the S381 position and on the corresponding site in TAZ primes both proteins for further phosphorylation events by CK18/c in the degradation motif, which then signals for interaction with the-TRCP E3 ubiquitin ligase, leading to polyubiquitination and degradation of YAP.

In some instances, Lats1/2 phosphorylates TAZ at the [HXRXXS] consensus motifs. TAZ comprises four [HXRXXS] consensus motifs, wherein X denotes any amino acid residues. In some instances, Lats1/2 phosphorylates TAZ at one or more of the consensus motifs. In some instances, Lats1/2 phosphorylates TAZ at all four of the consensus motifs. In some instances, Lats1/2 phosphorylate at the S89 amino acid position. The phosphorylation of TAZ S89 promotes 14-3-3 protein binding and results in cytoplasmic sequestration of TAZ. Mutation of TAZ at the S89 position thereby disrupts its interaction with 14-3-3 and subsequently promotes nuclear translocation.

In some embodiments, phosphorylated YAP/TAZ accumulates in the cytoplasm, and undergoes SCF^{β-TRCP}-mediated ubiquitination and subsequent proteasomal degradation. In some instances, the Skp, Cullin, F-box containing complex (SCF complex) is a multi-protein E3 ubiquitin ligase complex that comprises a F-box family member protein (e.g. Cdc4), Skp1, a bridging protein, and RBXI which contains a small RING Finger domain which interacts with E2-ubiquitin conjugating enzyme. In some cases, the F-box family comprises more than 40 members, in which exemplary members include F-box/WD repeat-containing protein IA (FBXWIA, βTrCP1, Fbxw1, hsSlimb, plkappaBalpha-E3 receptor subunit) and S-phase kinase-associated proteins 2 (SKP2). In some embodiments, the SCF complex (e.g. SCP^{βTrCP1}) interacts with an E1 ubiquitin-activating enzyme and an E2 ubiquitin-conjugating enzyme to catalyze the transfer of ubiquitin to the YAP/TAZ substrate. Exemplary E1 ubiquitin-activating enzymes include those encoded by the following genes: UBAI, UBA2, UBA3, UBA5, UBA5, UBA7, ATG7, NAEI, and SAEI Exemplary E2 ubiquitin-conjugating enzymes include those encoded by the following genes: UBE2A, UBE2B, UBE2C, UBE2DI, UBE2D2, UBE2D3, UBE2EI, UBE2E2, UBE2E3, UBE2F, UBE2GI, UBE2G2, UBE2H, UBE21, UBE2JI, UBE2J2, UBE2K, UBE2L3, UBE2L6, UBE2M, UBE2N, UBE20, UBE2QI, UBE2Q2, UBE2RI, UBE2R2, UBE2 UBE2T, UBE2U, UBE2VI, UBE2V2, UBE2ZATG2, BIRC5, and UFCL In some embodiments, the ubiquitinated YAP/TAZ further undergoes the degradation process through the 26S proteasome.

In some embodiments, the Hippo pathway is regulated upstream by several different families of regulators. In some instances, the Hippo pathway is regulated by the G-protein and its coupled receptors, the Crumbs complex, regulators upstream of the MST kinases, and the adherens junction.

YAP/TAZ Interaction with TEAD

In some embodiments, un-phosphorylated and/or dephosphorylated YAP/TAZ accumulates in the nucleus. Within the nucleus, YAP/TAZ interacts with the TEAD family of transcription factors (e.g. TEADI, TEAD2, TEAD3, or TEAD4) to activate genes involved in anti-apoptosis and proliferation, such as for example CTFG, Cyr61, and FGFI

In some embodiments, the compounds disclosed herein modulate the interaction between YAP/TAZ and TEAD. In some embodiments, the compounds disclosed herein bind to TEAD, YAP, or TAZ and prevent the interaction between YAP/TAZ and TEAD.

The present invention also provides a method for the synthesis of compounds useful as intermediates in the preparation of compounds of the invention.

The compounds described herein can be prepared according to the procedures of the following schemes and examples, using appropriate materials and are further exemplified by the following specific examples. Deuterated versions of the compounds of the invention can be prepared by substituting an appropriate isotopically labeled reagent for a non-isotopically labeled reagent. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. Reagents and starting materials for preparing the intermediates and example compounds are commercially available, unless indicated otherwise. All temperatures are degrees Celsius unless otherwise noted. Mass spectra (MS) were measured by electrospray ion-mass spectroscopy (ESI). ¹H NMR spectra were recorded at 300-500 MHz.

### Schemes and Examples

The abbreviations used herein have the following tabulated meanings. Abbreviations not tabulated below have their meanings as commonly used unless specifically stated otherwise.

| | |
|---|---|
| ACN | acetonitrile |
| AcOH | acetic acid |
| aq | aqueous |
| Boc | tert-butoxycarbamate |
| Boc₂O | di-tert-butyl dicarbonate |
| BPD | bis(pinacolato)diboron |
| BTMG | 2-tert-butyl-1,1,3,3-tetramethylguanidine |
| Bu₃SnCl | tributyltin chloride |
| ° C. | degrees Celsius |
| calc'd | Calculated |
| CDCl₃ | deuterated chloroform |
| CD₃OD | deuterated methanol |
| conc | concentrated |
| DAST | Diethylaminosulfur trifluoride |
| DCM | Dichloromethane |
| DCE | dichloroethane |
| DIEA | Diisopropylethylamine |
| DMA | Dimethylacetamide |
| DMF | Dimethylformamide |
| DMSO | Dimethylsulfoxide |
| eq or equiv | equivalents |
| ESI | electron spray ionization |
| EtOAc | ethyl acetate |
| EtOH | Ethanol |
| h | hours |
| ¹H NMR | proton nuclear magnetic resonance (data) |
| HPLC | high performance liquid chromatography |
| IC₅₀ | inhibitory concentration at 50% maximum response |
| LCMS | liquid chromatography mass spectrometry |
| MeCN | acetonitrile |
| MeOH | methanol |
| MHz | Megahertz |
| min | minutes |
| MS | mass spectrum (data) |
| NaOAc | sodium acetate |
| NBS | N-bromosuccinimide |
| NMR | nuclear magnetic resonance (data) |
| KOAc | potassium acetate |
| K₃PO₄ | potassium phosphate |
| Pd₂(dba)₃ | tris(dibenzylideneacetone)dipalladium(0) |
| PdCl₂(dppf) | [1,1′-bis(diphenylphosphino)ferrocene]dichloropalladium(II) |
| PdCl₂(dtbpf) | [1,1′-bis(di-tert-butylphosphino)ferrocene] |
| | dichloropalladium(II) |
| PdCl₂(PPh₃)₄ | bis(triphenylphosphine)palladium(II)dichloride |
| P(NMe₂)₃ | tris(dimethylamino)phosphine |
| RT | room temperature |
| sat | saturated |
| SFC | supercritical fluid chromatography |
| TEA | triethylamine |
| TFA | trifluoroacetic acid |
| TFAA | Trifluoroacetic anhydride |
| THF | tetrahydrofuran |
| TLC | thin layer chromatography |
| % wt | weight percentage |
| XPhos | 2-dicyclohexylphosphino-2′,4′,6′-triisopropylbiphenyl |
| XPhos-Pd- | chloro(2-dicyclohexylphosphino-2′,4′,6′-triisopropyl-1,1′- |
| G2 | biphenyl)[2-(2′-amino-1,1′-biphenyl)]palladium(II) |
| XPhos-Pd- | methanesulfonato(2-dicyclohexylphosphino-2′,4′,6′- |
| G3 | triisopropyl-1,1′-biphenyl)[2-(2′-amino-1,1′-biphenyl)] |
| | palladium(II) |
| XPhos-Pd- | methanesulfonato(2-dicyclohexylphosphino-2′,4′,6′- |
| G4 | triisopropyl-1,1′-biphenyl)[2-(2′-methylamino-1,1′-biphenyl)] |
| | palladium(II) |

### Methods of Synthesis

The compounds in the present invention can be prepared according to the following general schemes using appropriate materials, and are further exemplified by the subsequent specific examples. The compounds illustrated in the examples are not to be construed as forming the only genus that is considered as the invention. The illustrative examples below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of the instant invention herein above.

Those skilled in the art will readily understand that known variations of the conditions and processes of the following preparative procedures can be used to prepare these compounds. The invention will now be illustrated in the following non-limiting Examples in which, unless otherwise stated. All reactions were stirred (mechanically, stir bar/stir plate, or shaken) and conducted under an ambient (air) atmosphere unless specifically stated otherwise. All temperatures are degrees Celsius (° C.) unless otherwise noted. Ambient temperature is 15-25° C. Most compounds were purified by reverse-phase preparative HPLC, MPLC on silica gel, recrystallization and/or trituration (suspension in a solvent followed by filtration of the solid). The course of the reactions was followed by thin layer chromatography (TLC) and/or LCMS and/or NMR and reaction times are given for illustration only. All end products were analyzed by NMR and LCMS. Intermediates were analyzed by NMR and/or TLC and/or LCMS.

### General Syntheses

The compounds of formula I may be prepared by methods known in the art of organic synthesis as set forth in part by the following synthetic schemes and synthetic procedures and conditions for the illustrative intermediates and examples.

The compounds described herein may be made from commercially available starting materials or synthesized using known organic, inorganic, and/or enzymatic processes.

While the present invention has been described in conjunction with the specific examples set forth below, many alternatives, modifications, and variations thereof will be apparent to those of ordinary skill in the art. All such alternatives, modifications, and variations are intended to fall within the spirit and scope of the present invention.

### General Synthetic Schemes

Several synthetic methods were employed to access the compounds described herein. Final compounds were evaluated for biological activity in the assays in either the neutral form, as a TFA salt, or as a HCl salt, and were screened in the activity assay as either the racemate or as resolved enantiomers and diastereomers. The chiral separations were conducted on either the final compounds or on a synthetic intermediate. Chiral separation conditions (if applicable) are noted where appropriate.

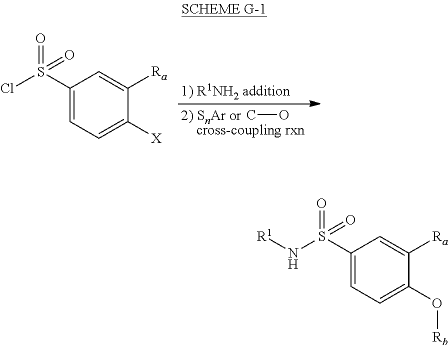

The general synthetic approaches are outline in Scheme G-1-3. In the first step of Scheme G-1, the sulfonamide group (R¹) is installed from commercial material, such as 3-bromo-4-fluorobenzene-1-sulfonyl chloride. In step two, oxygen nucleophiles (R_{b}) can be installed via S_{N}Ar reaction under basic conditions, displacing the preinstalled halogen (X), or coerced under C—O metal-mediated cross-coupling conditions. The installation of the oxygen-containing group can be installed early or later in the synthesis, depending on the needs of the operator.

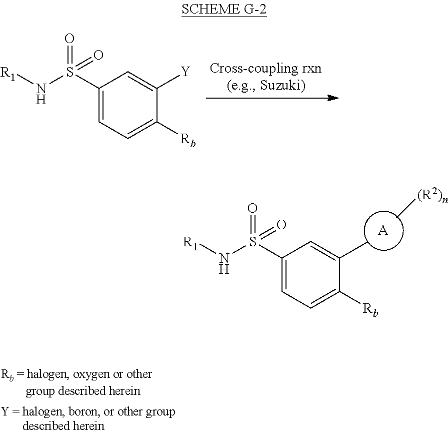

Scheme G-2 describes the general methods used to install the (hetero)aryl portion of the molecule. While not exclusive, the most common reaction employed are C—C cross-couplings reactions (e.g., Suzuki, Stille reaction, etc.) using Pd or Cu catalysts, beginning from 3-bromo-4-fluoro-N-methylbenzenesulfonamide, for example. For C—N bond forming reactions, Cu cross-couplings conditions can be employed, for example. The installation of the ring system can be installed early or later in the synthesis, depending on the needs of the operator.

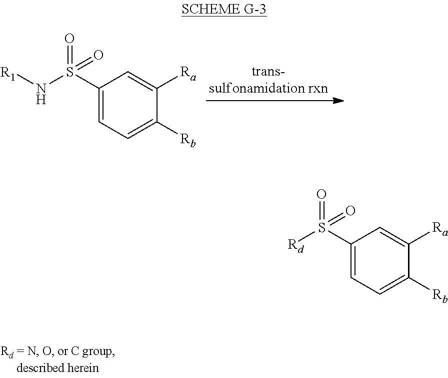

Scheme G-3 describes the general method(s) used to install and/or modify the sulfonamide or sulfone portion of the compound using reported conditions.

### Synthesis of Common Intermediates

### Intermediate I-1. Preparation of 3-bromo-4-fluoro-N-methylbenzenesulfonamide

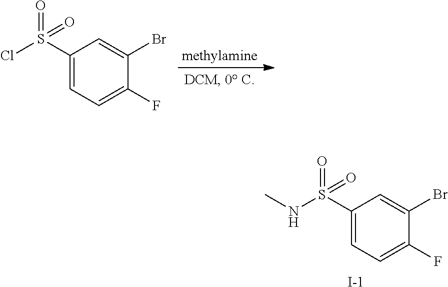

To a solution of 3-bromo-4-fluorobenzene-1-sulfonyl chloride (5 g, 18 mmol) in DCM (35 mL) was added methylamine (5.10 g, 54.8 mmol) at 0° C. The resulting mixture was stirred at 20° C. for 1 h. The reaction was quenched by addition of H₂O (100 mL). The combined organic layers were washed with brine (20 mL), dried (Na₂SO₄), filtered, and concentrated under reduced pressure to afford 3-bromo-4-fluoro-N-methylbenzenesulfonamide. MS (ESI) m/z calc'd for C₇H₈BrFNO₂S [M+1]⁺268 and 270, found 268 and 270. ¹H NMR (400 MHz, CDCl₃-d) δ 8.08 (dd, J=6.1, 2.1 Hz, 1H), 7.80 (ddd, J=8.6, 4.3, 2.4 Hz, 1H), 7.22-7.28 (m, 2H), 2.68 (d, J=5.0 Hz, 3H).

### Intermediate I-2. Preparation of 3-bromo-N-methyl-4-(4-(trifluoromethyl)phenoxy) benzenesulfonamide

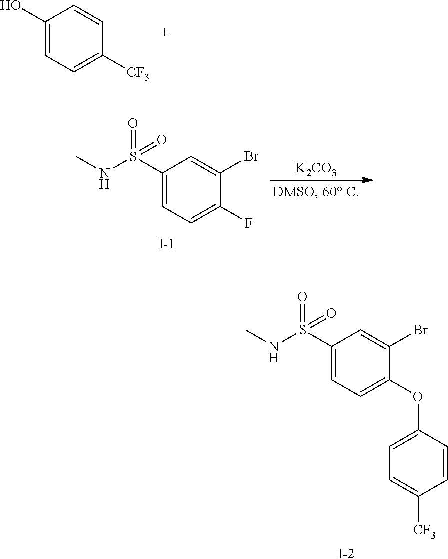

To a solution of 4-(trifluoromethyl)phenol (2.42 g, 14.9 mmol) in DMSO (20 mL) was added K2CO₃ (3.09 g, 22.4 mmol) portionwise. The reaction mixture was stirred for 1 min. To this reaction mixture was added 3-bromo-4-fluoro-N-methylbenzenesulfonamide (2.0 g, 7.5 mmol). The reaction mixture was stirred at 60° C. for 14 h. The reaction was cooled to RT and partitioned with ethyl acetate (100 mL) and water (50 mL). The organic layer was washed with brine (40 mL, ×3), dried (Na₂SO₄), filtered and concentrated under reduced pressure. The residue was purified by flash silica gel chromatography (ethyl acetate/hexanes, 0-50%) to afford 3-bromo-N-methyl-4-(4-(trifluoromethyl)phenoxy)benzenesulfonamide. MS (ESI) m z C₁₄H₁₂BrF₃NO₃S [M+1] calc'd 410 and 412, found 410 and 412. ¹H NMR (400 MHz, CDCl₃-d) δ 8.18 (d, J=2.4 Hz, 1H), 7.79 (dd, J=8.6, 2.0 Hz, 1H), 7.67 (d, J=8.6 Hz, 2H), 7.11 (d, J=8.6 Hz, 2H), 7.04 (d, J=8.6 Hz, 1H), 2.74 (d, J=5.1 Hz, 3H).

Each of the elaborated bromides presented in Table 1 below were prepared in accordance with the synthetic routes in Intermediate I-2, using procedures analogous to those described above.

TABLE 1

| Intermediate | Structure |
|---|---|
| I-3 | 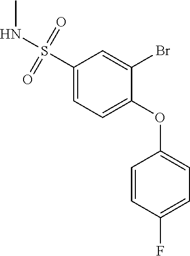 [SMILES: CNS(=O)(=O)c1ccc(Oc2ccc(F)cc2)c(Br)c1] |
| I-4 | 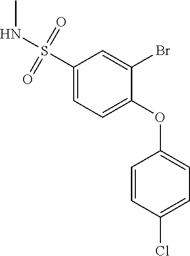 [SMILES: CNS(=O)(=O)c1ccc(Oc2ccc(Cl)cc2)c(Br)c1] |
| I-5 | 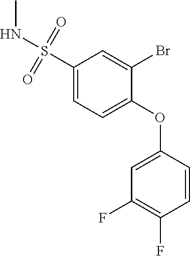 [SMILES: CNS(=O)(=O)c1ccc(Oc2ccc(F)c(F)c2)c(Br)c1] |
| I-6 | 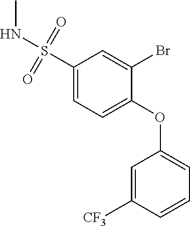 [SMILES: CNS(=O)(=O)c1ccc(Oc2cccc(C(F)(F)F)c2)c(Br)c1] |
| I-7 | 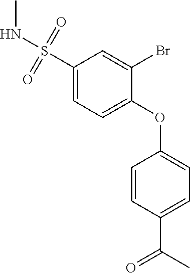 [SMILES: CNS(=O)(=O)c1ccc(Oc2ccc(C(C)=O)cc2)c(Br)c1] |

### Intermediate I-8. Preparation of (5-(N-methylsulfamoyl)-2-(4-(trifluoromethyl)phenoxy) phenyl)boronic acid

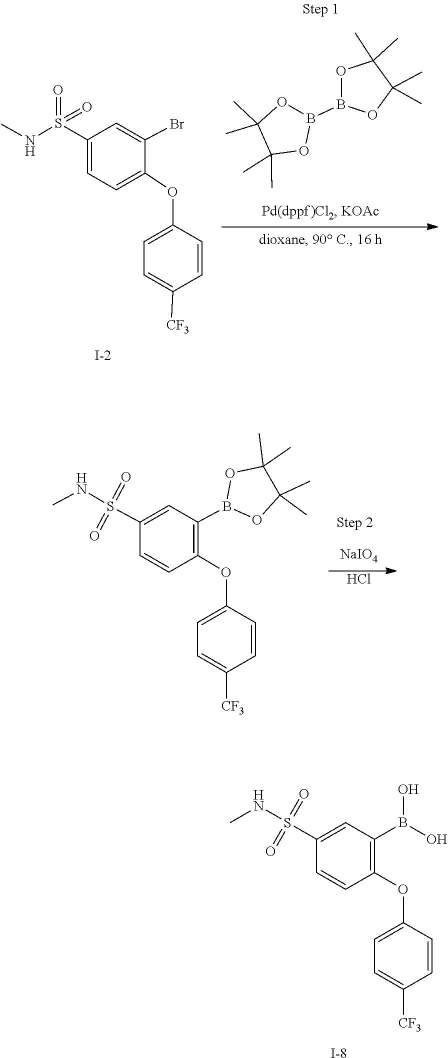

Step 1. A mixture of 3-bromo-N-methyl-4-(4-(trifluoromethyl)phenoxy)benzenesulfonamide 1-2 (1.0 g, 2.4 mmol), potassium acetate (0.479 g, 4.88 mmol), PdCl₂(dppf) (0.089 g, 0.12 mmol) and bis(pinacolato)diboron (1.55 g, 6.09 mmol) in dioxane (40 mL) was degassed and backfilled with N₂ (×3). The mixture was heated to 100° C. for 16 h. The mixture was cooled to RT, filtered, and the filtrate was concentrated under reduced pressure. The reaction was quenched by addition of H₂O (100 mL), and the aqueous phase was extracted with EtOAc (50 mL, ×3). The combined organic layers were washed with brine (20 mL), dried (Na₂SO₄), filtered and concentrated under reduced pressure. The residue was purified by flash silica gel chromatography (ethyl acetate/hexanes, 0-50%) to afford N-methyl-3-(4,4,5,5-tetramethyl-1,3,2-dioxaborolan-2-yl)-4-(4-(trifluoromethyl)phenoxy) benzenesulfonamide. MS (ESI) m z C₂₀H₂₄BF₃NO₅S [M+1]+ calc'd 458, found 458.

Step 2. To a solution of N-methyl-3-(4,4,5,5-tetramethyl-1,3,2-dioxaborolan-2-yl)-4-(4-(trifluoromethyl)phenoxy)benzenesulfonamide (5.81 g, 12.7 mmol) in THF (60 mL) and water (15 mL) was added sodium periodate (8.14 g, 38.1 mmol) portionwise at 15° C. over 30 min. After stirring for 30 min at 15° C., HCl (1 N in water, 7.7 mL, 7.7 mmol) was added to the mixture at 15° C. The resulting mixture was stirred for another 16 h. The reaction was quenched by addition of H₂O (20 mL), and the aqueous phase was extracted with EtOAc (10 mL, ×3). The combined organic layers were washed with brine (10 mL), dried (Na₂SO₄), filtered, and concentrated under reduced pressure. The residue was purified by preparative HPLC (reverse phase, C₁₈, ACN/water with 0.1% TFA modifier) to afford (5-(N-methylsulfamoyl)-2-(4-(trifluoromethyl)phenoxy)phenyl)boronic acid. MS (ESI) m/z C₁₄H₁₄BF₃NO₅S [M+1]+ calc'd 376, found 376. ¹H NMR (400 MHz, DMSO-d₆) δ 8.00 (d, J=2.2 Hz, 1H), 7.78 (dd, J=2.4, 8.6 Hz, 1H), 7.71 (d, J=8.4 Hz, 2H), 7.21-7.13 (m, 2H), 7.03 (d, J=8.7 Hz, 1H), 2.45 (s, 2H), 2.46-2.44 (m, 1H).

Intermediate I-9. Preparation of 3-(6,7-dihydro-5H-pyrrolo[1,2-α]imidazol-2-yl)-4-fluoro-N-methylbenzenesulfonamide

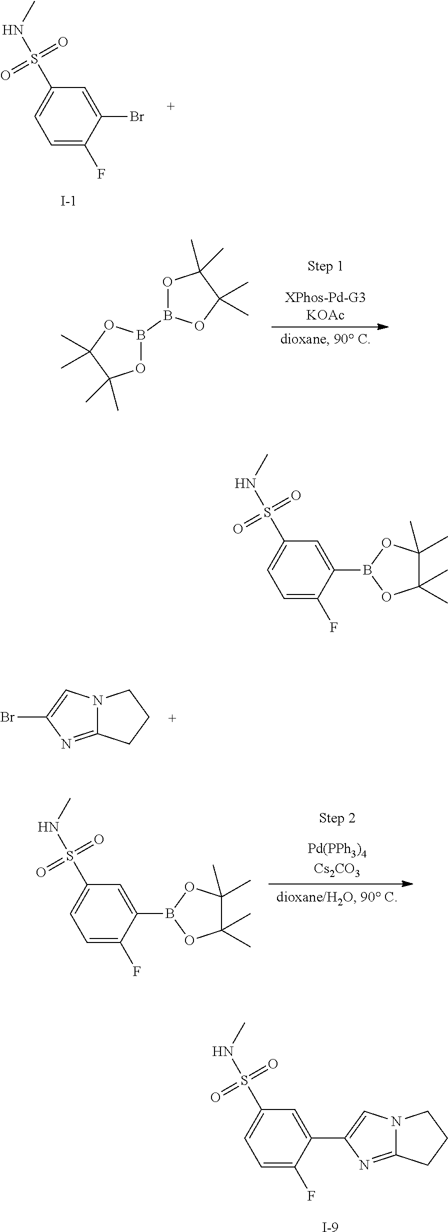

Step 1. A mixture of 3-bromo-4-fluoro-N-methylbenzenesulfonamide (100 mg, 0.37 mmol), bis(pinacolato)diboron (142 mg, 0.56 mmol), XPhos-Pd-G3 (15.8 mg, 0.0192 mmol), and potassium acetate (73.2 mg, 0.751 mmol) were combined in dioxane (1.87 mL). The reaction vessel was purged with a needle of N₂ for 5 min. The vial was sealed and heated to 90° C. for 45 min. The mixture was cooled to RT, concentrated under reduced pressure, and diluted with sat NH₄Cl. The aqueous phase was extracted with EtOAc (×3). The combined organic layers were dried over Na₂SO₄, concentrated under reduced pressure, and dried under high vacuum. The residue was a mixture of the boronic acid and pinacolate boronate ester.

Step 2. A mixture of 4-fluoro-N-methyl-3-(4,4,5,5-tetramethyl-1,3,2-dioxaborolan-2-yl)benzenesulfonamide and the boronic acid (100 mg, 0.32 mmol), 2-bromo-6,7-dihydro-5H-pyrrolo[1,2-α]imidazole (104 mg, 0.56 mmol), Cs₂CO₃ (310 mg, 0.95 mmol), and Pd(PPh₃)₄ (37 mg, 0.032 mmol) in dioxane (3.47 mL) and H₂O (496 μL)was purged with a needle of N₂ for 4 min. The vial was sealed and heated to 90° C. for 60 min. The mixture was cooled to RT and diluted with MeOH (1 mL), filtered, concentrated, and purified by flash silica gel chromatography (3:1 ethyl acetate/EtOH in hexanes, 0-50%) to afford 3-(6,7-dihydro-5H-pyrrolo[1,2-α]imidazol-2-yl)-4-fluoro-N-methylbenzenesulfonamide. MS (ESI) m/z C₁₃H₁₅FN₃O₂S [M+1]+ calc'd 296, found 296.

Intermediate I-10. Preparation of 6-bromo-2-methyl-2,3-dihydroimidazo[2,1-b]oxazole

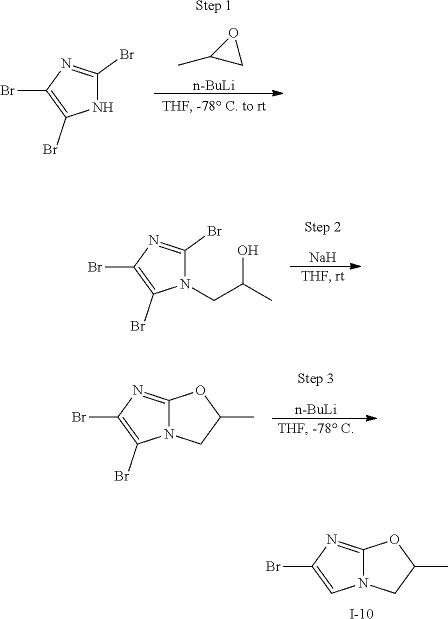

Step 1: To a solution of 2,4,5-tribromo-1H-imidazole (1.0 g, 3.3 mmol) in THF (20 mL), was added dropwise n-butyllithium (0.210 g, 3.28 mmol) at −78° C. After 0.75 h, 2-methyloxirane (1.91 g, 32.8 mmol) was added and the reaction mixture was warmed from −78° C. to 20° C., and left to stir for 48 h. The reaction was quenched with sat. NH₄Cl (50 mL). The aqueous phase was extracted with EtOAc (3×30 mL), and the combined organic fractions were dried and concentrated under reduced pressure. The residue was purified by column chromatography (0-50% EtOAc in petroleum ether) to afford 1-(2,4,5-tribromo-1H-imidazol-1-yl)propan-2-ol. MS (ESI) m/z C₆HsBr₃N₂O [M+1]⁺ calc'd 363, 365, found 363, 365.

Step 2: NaH (66.1 mg, 1.65 mmol) was added to a stirred mixture of 1-(2,4,5-tribromo-1H-imidazol-1-yl)propan-2-ol (300 mg, 0.827 mmol) in DMF (2 mL) at 0° C. and the mixture was warmed with stirring to 25° C. for 16 h. The reaction was quenched with water (20 mL) at 0° C., and the aqueous phase was extracted with EtOAc (3×10 mL). The combined organic fractions were dried and concentrated under reduced pressure to afford crude 5,6-dibromo-2-methyl-2,3-dihydroimidazo[2,1-b]oxazole. MS (ESI) m/z C₆H₇Br₂N₂O [M+1]+ calc'd 283, found 283. ¹H NMR (400 MHz, CDCl₃) δ 5.31-5.46 (m, 1H), 4.21 (dd, J=9.20, 8.02 Hz, 1H), 3.70 (dd, J=9.39, 7.83 Hz, 1H), 1.63 (d, J=6.26 Hz, 3H).

Step 3: n-Butyllithium (0.170 ml, 0.426 mmol) was added to a stirred solution of 5,6-dibromo-2-methyl-2,3-dihydroimidazo[2,1-b]oxazole (100 mg, 0.355 mmol) in THF (4 mL) at −78° C. and the mixture was left to stir at −78° C. for 2 h. The reaction was quenched with NH₄Cl (20 mL) and the aqueous phase was extracted with EtOAc (3×10 mL). The combined organic fractions were dried and concentrated under reduced pressure to afford crude 6-bromo-2-methyl-2,3-dihydroimidazo[2,1-b]oxazole. MS (ESI) m/z C₆HsBrN₂O [M+1]⁺ calc'd 203, 205, found 203, 205.

### Intermediate I-11. Preparation of 2-bromo-6-methyl-6,7-dihydro-5H-pyrrolo[1,2-α]imidazole

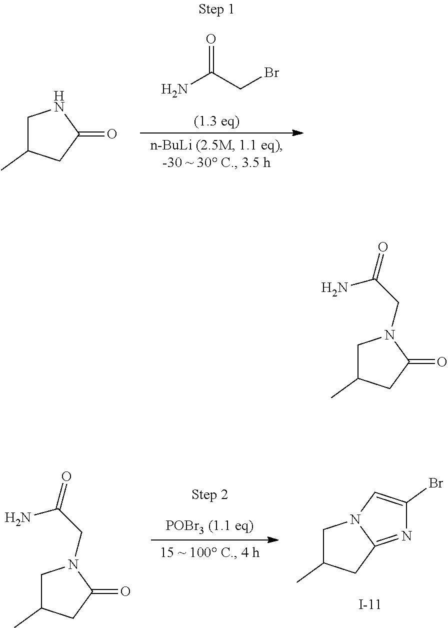

Step 1: To a mixture of 4-methylpyrrolidin-2-one (5.0 g, 50 mmol) in dry THF (50 mL) was added n-BuLi (2.5 M, 22.2 mL, 55.5 mmol) portionwise over a period of 30 min at −30° C. The mixture was stirred at this temperature for 30 min. 2-Bromoacetamide (9.1 g, 66 mmol) in THF (90 mL) was added portionwise over a period of 30 min at −30° C. The mixture was warmed to 15° C. for 2 h. The reaction solution was poured into sat. NH₄Cl (20 mL) at 0° C., and the mixture was concentrated under vacuum. The crude product was purified by preparative HPLC (reverse phase, C₁₈, ACN/water with 0.1% TFA modifier) to afford 2-(4-methyl-2-oxopyrrolidin-1-yl)acetamide. MS (ESI) m/z C₇H₁₃N₂O₂ [M+1]⁺ calc'd 157, found 157.

Step 2: POBr₃ (6.1 g, 21 mmol) was warmed to 60° C., and 2-(4-methyl-2-oxopyrrolidin-1-yl)acetamide (3.0 g, 19 mmol) was added. The mixture was heated to 100° C. for 3 h. The reaction mixture was poured into sat. NaHCO₃ (100 mL) at 15° C., and the aqueous layer was extracted with CH₂Cl₂ (100 mL). The organic layer was washed with brine (100 mL), dried over Na₂SO₄, and concentrated under reduced pressure. The residue was purified by silica gel chromatography (50:1-1:1 Petroleum ether/Ethyl acetate) to afford 2-bromo-6-methyl-6,7-dihydro-5H-pyrrolo[1,2-a]imidazole. MS (ESI) m/z C₇HioBrN₂ [M+1]⁺ calc'd 201, 203, found 201, 203. ¹H (400 MHz, CD₃OD) δ 6.92 (s, 1H), 4.04-4.09 (m, 1H), 3.47-3.51 (m, 1H), 2.88-2.98 (m, 2H), 2.31-2.37 (m, 2H), 1.14 (d, J=6.8 Hz, 3H).

### Intermediate I-12. Preparation of 2-bromo-7-methyl-6,7-dihydro-5H-pyrrolo[1,2-a]imidazole

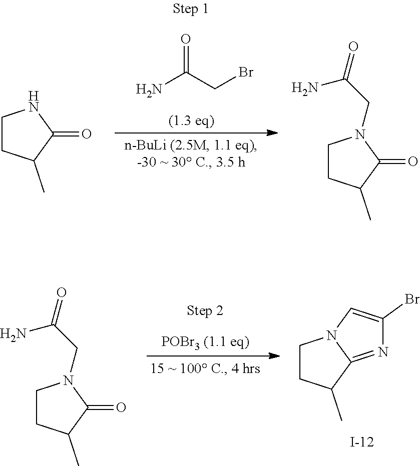

Step 1: To a mixture of 3-methylpyrrolidin-2-one (5.0 g, 50 mmol) in dry THF (50 mL) was added n-BuLi (2.5 M, 22.2 mL, 55.5 mmol) portionwise over a period of 30 min at −30° C. The mixture was stirred at this temperature for 30 min. 2-Bromoacetamide (9.1 g, 66 mmol) in THF (90 mL) was added portionwise over a period of 30 min at −30° C. The mixture was warmed to 15° C. for 2 h. The reaction solution was poured into sat. NH₄Cl (20 mL) at 0° C., and the mixture was concentrated under vacuum. The residue was purified by preparative HPLC (reverse phase, C₁₈, ACN/water with 0.1% TFA modifier) to afford 2-(3-methyl-2-oxopyrrolidin-1-yl)acetamide. MS (ESI) m/z C₇H₁₃N₂O₂ [M+1]⁺ calc'd 157, found 157.

Step 2: POBr₃ (27.5 g, 96.0 mmol) was warmed to 60° C., and 2-(3-methyl-2-oxopyrrolidin-1-yl)acetamide (10.0 g, 64.0 mmol) was added. The mixture was heated to 100° C. for 3 h. The reaction mixture was poured into sat. NaHCO₃ (100 mL) at 15° C., and the aqueous layer was extracted with CH₂Cl₂ (100 mL). The organic layer was washed with brine (100 mL), dried over Na₂SO₄, and concentrated under reduced pressure. The crude product was purified by silica gel chromatography (50:1-0:1 Petroleum ether:Ethyl acetate) to afford 2-bromo-7-methyl-6,7-dihydro-5H-pyrrolo[1,2-a]imidazole. MS (ESI) m/z C₇HioBrN₂ [M+1]⁺ calc'd 201, 203, found 201, 203. ¹H NMR (400 MHz, CD₃OD) δ 7.02 (s, 1H), 4.03-4.09 (m, 1H), 3.90-3.97 (m, 1H), 3.12-3.19 (m, 1H), 2.74-2.82 (m, 1H), 2.09-2.18 (m, 1H), 1.31 (d, J=6.8 Hz, 3H).

### Intermediate I-13. Preparation of 6-bromo-2,3-dihydro-1H-imidazo[1,2-b]pyrazole

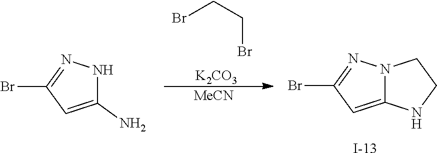

K2CO₃ (341 mg, 2.47 mmol) was added to a stirred mixture of 3-bromo-1H-pyrazol-5-amine (200 mg, 1.24 mmol) and 1,2-dibromoethane (232 mg, 1.24 mmol) in MeCN (10 ml) at 20° C. The mixture was heated with stirring at 80° C. for 16 h. The reaction mixture was cooled to 20° C. and filtered, and the filtrate was concentrated under reduced pressure. The residue was purified by preparative HPLC (reverse phase, C₁₈, ACN/water with 0.05% NH₃ modifier) to afford 6-bromo-2,3-dihydro-1H-imidazo[1,2-b]pyrazole. MS (ESI) m/z C₅H₇BrN₃ [M+1]⁺ calc'd 188, 190, found 188, 190. ¹H NMR (400 MHz, CDCl₃) δ 5.45 (s, 1H), 4.14-4.21 (m, 2H), 3.93-3.99 (m, 2H).

### Intermediate I-14. Preparation of 7-bromo-2,3-dihydropyrazolo[5,1-b]oxazole

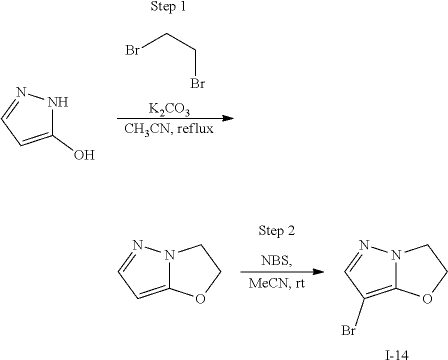

Step 1: K2CO₃ (1.09 g, 7.85 mmol) was added to a stirred mixture of 1H-pyrazol-5-ol (200 mg, 2.38 mmol) and 1,2-dibromoethane (1.48 g, 7.85 mmol) in MeCN (10 mL) at 20° C. The mixture was heated with stirring at 80° C. for 16 h. The reaction mixture was cooled to 20° C. and filtrated, the filtrate was concentrated under reduced pressure. The residue was purified by column chromatography (0-50% EtOAc in petroleum ether) to afford 2,3-dihydropyrazolo[5,1-b]oxazole. ¹H NMR (400 MHz, CDCl₃) δ 7.37 (d, J=1.56 Hz, 1H), 5.35 (d, J=1.96 Hz, 1H), 5.02-5.09 (m, 2H), 4.29 (t, J=7.83 Hz, 2H).

Step 2: To a solution of 2,3-dihydropyrazolo[5,1-b]oxazole (320 mg, 2.91 mmol) in MeCN (10 mL) was added NBS (1.29 g, 7.27 mmol). The reaction mixture was stirred at 25° C. for 48 h.

The reaction mixture was concentrated, and the residue was purified by column chromatography (0-50% EtOAc in petroleum ether) to afford 7-bromo-2,3-dihydropyrazolo[5,1-b]oxazole. MS (ESI) m/z C₅H₆BrN₂₀ [M+1]⁺ calc'd 189, 191, found 189, 191. ¹H NMR (400 MHz, CDCl₃) δ 7.53 (s, 1H), 7.31 (s, 1H), 5.08-5.15 (m, 2H), 4.35 (t, J=8.02 Hz, 2H).

### Intermediate I-15. Preparation of 4-(4-fluorophenoxy)-N-methyl-3-(4,4,5,5-tetramethyl-1,3,2-dioxaborolan-2-yl)benzenesulfonamide

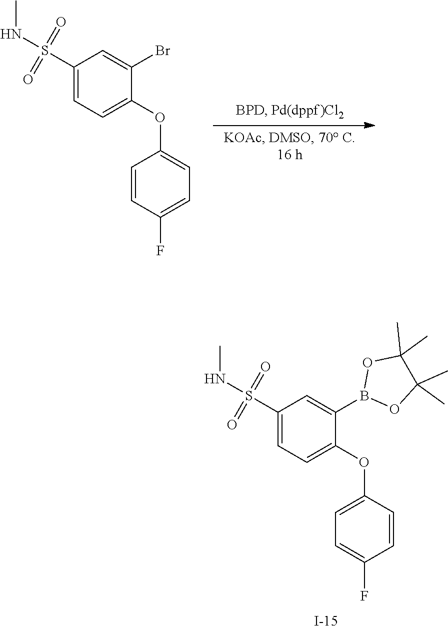

A mixture of 3-bromo-4-(4-fluorophenoxy)-N-methylbenzenesulfonamide (250 mg, 0.694 mmol), KOAc (136 mg, 1.39 mmol), PdCl₂(dppf) (25.4 mg, 0.035 mmol), and bis(pinacolato)diboron (441 mg, 1.74 mmol) in dioxane (15 mL) was degassed and backfilled with N₂ (×3). The mixture was heated to 80° C. for 16 h. The reaction was quenched with H₂O (100 mL), and the aqueous phase was extracted with EtOAc (50 mL×3). The combined organic layers were washed with brine (50 mL), dried over anhydrous Na₂SO₄, filtered, and concentrated under reduced pressure. The residue was purified by flash silica gel chromatography (0-50% EtOAc in petroleum ether) to afford 4-(4-fluorophenoxy)-N-methyl-3-(4,4,5,5-tetramethyl-1,3,2-dioxaborolan-2-yl)benzenesulfonamide. ¹H NMR (400 MHz, CDCl₃) δ 8.25 (d, J=2.74 Hz, 1H), 7.74-7.95 (m, 1H), 7.03-7.08 (m, 2H), 6.94-7.00 (m, 2H), 6.90 (d, J=9.00 Hz, 1H), 2.64-2.72 (m, 3H), 1.28 (s, 12H).

Each of the elaborated boronates presented in Table 2 below were prepared in accordance with the synthetic routes in Intermediate I-15, using procedures analogous to those described above.

TABLE 2

| Intermediate | Structure |
|---|---|
| I-16 | 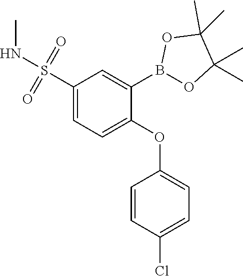 [SMILES: CNS(=O)(=O)c1ccc(Oc2ccc(Cl)cc2)c(B2OC(C)(C)C(C)(C)O2)c1] |
| I-17 | 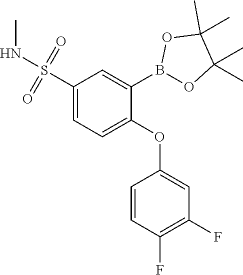 [SMILES: CNS(=O)(=O)c1ccc(Oc2ccc(F)c(F)c2)c(B2OC(C)(C)C(C)(C)O2)c1] |
| I-18 | 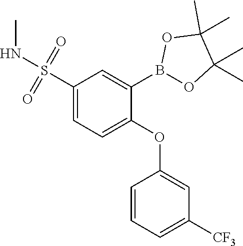 [SMILES: CNS(=O)(=O)c1ccc(Oc2cccc(C(F)(F)F)c2)c(B2OC(C)(C)C(C)(C)O2)c1] |
| I-19 | 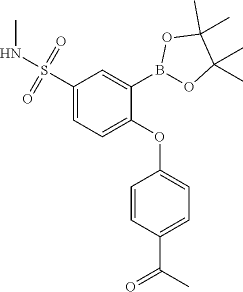 [SMILES: CNS(=O)(=O)c1ccc(Oc2ccc(C(C)=O)cc2)c(B2OC(C)(C)C(C)(C)O2)c1] |

### Intermediate I-20. Preparation of 2-(tributylstannyl)-5,6-dihydro-4H-pyrrolo[1,2-b]pyrazole

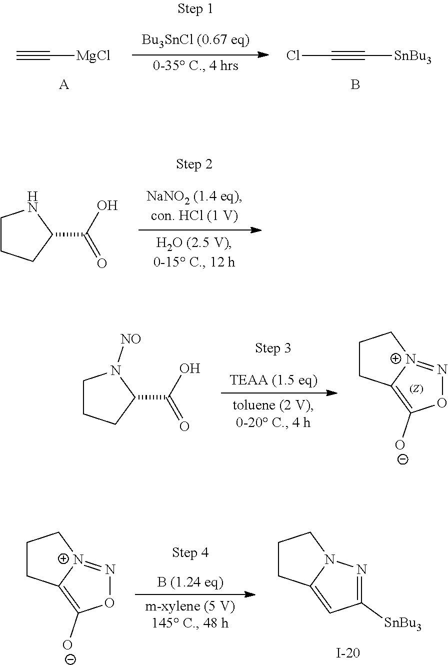

Step 1: To a solution of ethynyl magnesium chloride (0.5 M, 3.87 L, 1.94 mol) was added Bu₃SnCl (420 g, 1.29 mol) dropwise at 0° C. over 30 min. The mixture was stirred at 15° C. for 30 min and at 35° C. for 3 h under an N₂ atmosphere. The reaction was quenched by addition of 15% aq. NH₄Cl (1500 mL), and the aqueous phase was extracted with petroleum ether (2 L×3). The combined organic layers were washed with brine, dried over Na₂SO₄, filtered, and concentrated under reduced pressure to afford tributyl(chloroethynyl)stannane.

Step 2: A mixture of L-proline (300 g, 2.61 mol), NaNO₂ (252 g, 3.65 mol), H₂O (750 mL) was degassed and purged with N₂ (three times). The reaction mixture was cooled to 0° C., and then HCl (12 M, 282 mL) was added dropwise at 0° C. over 30 min. The mixture was stirred in the cold bath for 30 min, and then warmed slowly to 15° C. The mixture was stirred at 15° C. for 12 h under an N₂ atmosphere. The aqueous phase was extracted with EtOAc (700 mL×5). The combined organic layers were washed with brine, dried over Na₂SO₄, filtered, and concentrated under reduced pressure to afford nitroso-L-proline. MS (ESI) m/z C₅H₉N₂O₃ [M+1]⁺ calc'd 145, found 145.

Step 3: To a solution of nitroso-L-proline (347 g, 2.41 mol) in toluene (694 mL) was added dropwise TFAA (759 g, 3.61 mol) at 0° C. over 30 min. The mixture was stirred at 20° C. for 4 h. A mixture of K2CO₃ (532 g), deionized water (520 mL), and CH₂Cl₂ (694 mL) was added dropwise at 0° C., and the mixture was stirred at 20° C. for 1 h. The aqueous phase was extracted with DCM (700 mL×5). The combined organic layers were washed with brine, dried over Na₂SO₄, filtered, and concentrated under reduced pressure. The crude residue was purified by column chromatography (20/1-0/1 Petroleum ether/EtOAc) to afford 5,6-dihydro-4H-pyrrolo[1,2-c][1,2,3]oxadiazol-7-ium-3-olate. MS (ESI) m/z C₅H₇N₂O₂ [M+1]⁺ calc'd 127, found 127. ¹H NMR (400 MHz, CDCl₃) δ 4.45-4.35 (m, 2H), 2.91-2.82 (m, 2H), 2.81-2.70 (m, 2H).

Step 4: To a solution of 5,6-dihydro-4H-pyrrolo[1,2-c][1,2,3]oxadiazol-7-ium-3-olate (254 g, 2.01 mol) in m-xylene (1055 mL) was added tributyl(chloroethynyl)stannane (813 g, 2.50 mol). The mixture was stirred at 145° C. for 48 h. The residue was purified by column chromatography (I/O-15/1 Petroleum ether/EtOAc), and then re-purified by preparative HPLC (reverse phase, C18, ACN/water with 0.04% NH₃ modifier) to afford 2-(tributylstannyl)-5,6-dihydro-4H-pyrrolo[1,2-b]pyrazole. MS (ESI) m/z C₁₈H₃₅N₂Sn [M+1]⁺ calc'd 399, found 399. ¹H NMR (400 MHz, DMSO-d) δ 5.96 (s, 1H), 4.04 (t, J=7.2 Hz, 2H), 2.82-2.73 (m, 2H), 2.59-2.51 (m, 2H), 1.63-0.77 (m, 27H).

### Intermediate I-21. Preparation of 3-(6,7-dihydro-5H-pyrrolo[1,2-α]imidazol-2-yl)-4-hydroxy-N-methylbenzenesulfonamide

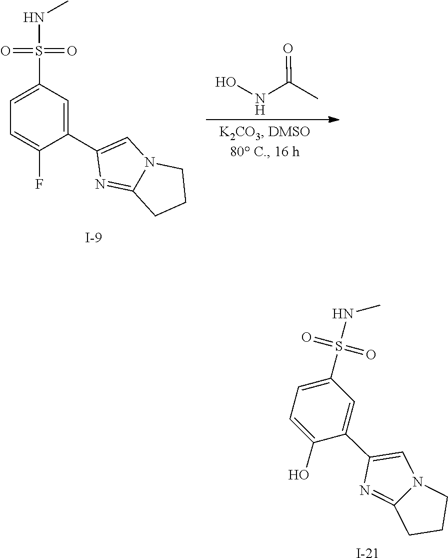

To a solution of 3-(6,7-dihydro-5H-pyrrolo[1,2-α]imidazol-2-yl)-4-fluoro-N-methylbenzenesulfonamide (I-9; 200 mg, 0.677 mmol) and K2CO₃ (468 mg, 3.39 mmol) in DMSO (12 mL) was added acetohydroxamic acid (153 mg, 2.03 mmol) under N₂. The reaction was stirred at 80° C. for 16 h. The reaction mixture was carefully quenched with 2 N HCl until pH ˜3-4. The aqueous phase was extracted with EtOAc (3×25 ml), dried over Na₂SO₄, filtered and concentrated. The residue was purified by flash silica gel chromatography (0-50% EtOAc in petroleum ether) to afford 3-(6,7-dihydro-5H-pyrrolo[1,2-α]imidazol-2-yl)-4-hydroxy-N-methylbenzenesulfonamide. MS (ESI) m/z C₁₃H₁₆N₃O₃S [M+1]⁺ calc'd 294, found 294.

### Intermediate I-22. Preparation of 5-(N-methylsulfamoyl)-2-(4-(trifluoromethyl)phenoxy) benzoic acid

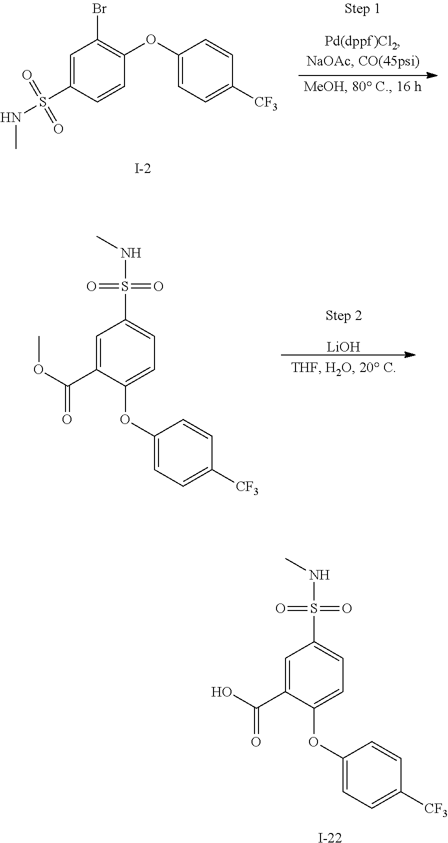

Step 1: To a solution of 3-bromo-N-methyl-4-(4-(trifluoromethyl)phenoxy)benzenesulfonamide (I-2; 200 mg, 0.49 mmol) in MeOH (10 mL) was added PdCl₂(dtbpf) (64 mg, 0.098 mmol) and NaOAc (80 mg, 0.98 mmol) under N₂ atmosphere. The mixture was degassed and backfilled with CO (three times). The resulting mixture was stirred under CO (45 psi) at 80° C. for 16 h.

The mixture was cooled to room temperature and filtered, and the filtrate was concentrated under reduced pressure. The residue was purified by preparative TLC (3:1 EtOAc in Pet. ether) to afford methyl 5-(N-methylsulfamoyl)-2-(4-(trifluoromethyl)phenoxy)benzoate. ¹H NMR (400 MHz, CDCl₃) δ 8.45 (d, J=2.35 Hz, 1H), 7.97 (dd, J=8.80, 2.54 Hz, 1H), 7.66 (d, J=8.22 Hz, 2H), 7.10 (d, J=8.61 Hz, 3H), 4.37 (br d, J=5.09 Hz, 1H), 3.87 (s, 3H), 2.74 (d, J=5.09 Hz, 3H).

Step 2: To a solution of methyl 5-(N-methylsulfamoyl)-2-(4-(trifluoromethyl)phenoxy)benzoate (0.2 g, 0.514 mmol) in THF (2 mL) were added LiOH (0.037 g, 1.5 mmol), water (0.4 mL) and MeOH (0.4 mL). The reaction mixture was stirred at 20° C. for 14 h. The reaction was partitioned with water (5 mL) and ethyl acetate (20 mL). The separated organic layer was washed with brine (10 mL×3), dried over anhydrous Na₂SO₄, filtered and concentrated under reduced pressure. The crude product was purified by preparative HPLC (reverse phase) to afford 5-(N-methylsulfamoyl)-2-(4-(trifluoromethyl)phenoxy)benzoic acid. MS (ESI) m/z C₁₅H₁₃F3NO₅S [M+1]⁺ calc'd 376, found 376. ¹H NMR (400 MHz, DMSO-d₆) δ 8.22 (d, J=1.96 Hz, 1H), 7.90-7.94 (m, 1H), 7.74 (d, J=8.61 Hz, 2H), 7.60 (br d, J=5.09 Hz, 1H), 7.32 (d, J=8.61 Hz, 1H), 7.15 (d, J=8.61 Hz, 2H), 2.45 (d, J=5.09 Hz, 3H).

### Intermediate I-23. Preparation of (5-(N-methylsulfamoyl)-2-(4-(trifluoromethyl)phenoxy)pyridin-3-yl)boronic acid

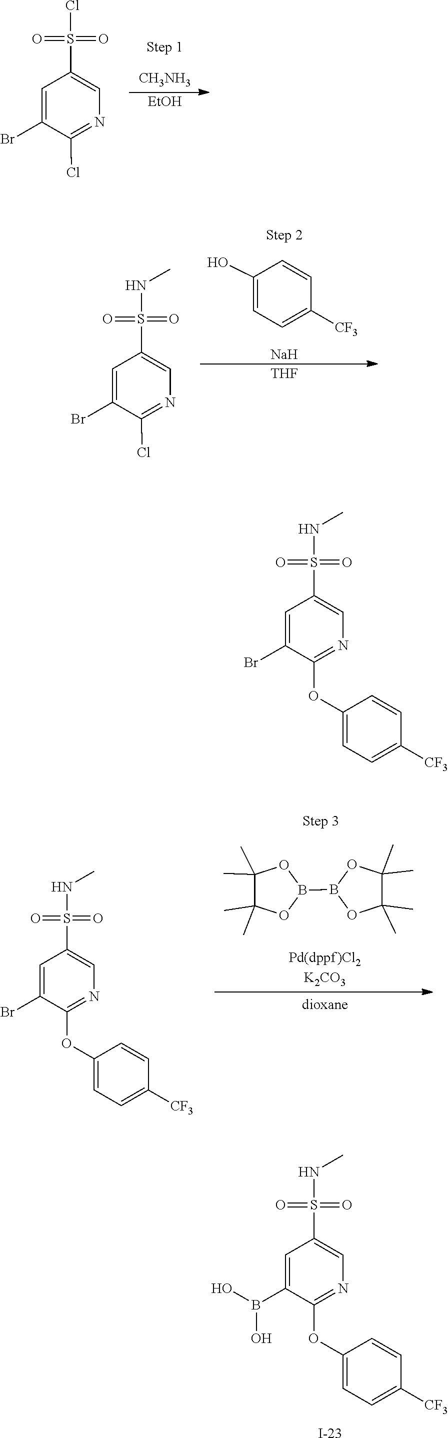

Step 1: To a solution of 5-bromo-6-chloropyridine-3-sulfonyl chloride (300 mg, 1.03 mmol) in CH₂Cl₂ (10 ml) was added methylamine (485 mg, 5.16 mmol) at 0° C. The reaction mixture was stirred at 20° C. for 1 h. The reaction was quenched with H₂O (100 mL). The combined organic layers were washed with brine (20 mL), dried over anhydrous Na₂SO₄, filtered and concentrated under reduced pressure to afford crude 5-bromo-6-chloro-N-methylpyridine-3-sulfonamide. ¹H NMR (400 MHz, CDCl₃) δ 8.78 (d, J=2.35 Hz, 1H), 8.36 (d, J=1.96 Hz, 1H), 2.78 (d, J=5.48 Hz, 3H).

Step 2: 4-(Trifluoromethyl) phenol (179 mg, 1.11 mmol), 5-bromo-6-chloro-N-methylpyridine-3-sulfonamide (200 mg, 0.700 mmol) and Cs₂CO₃ (434 mg, 1.33 mmol) were suspended in DMSO (5 mL). The mixture was heated to 80° C. for 1 h, and then cooled to RT. Water (10 mL) and ethyl acetate (50 mL) was added to the reaction mixture, and the layers were separated. The organic layer was washed with brine and dried over Na₂SO₄. Purification by column chromatography (3:1-1:1 Pet. ether/EtOAc) afforded 5-bromo-N-methyl-6-(4-(trifluoromethyl) phenoxy) pyridine-3-sulfonamide. MS (ESI) m/z C₁₃H₁₁BrF₃N₂O₃S [M+1]⁺ calc'd 411, 413, found 411, 413. ¹H NMR (400 MHz, CDCl₃) δ 8.50 (d, J=2.35 Hz, 1H), 8.38 (d, J=2.35 Hz, 1H), 7.73 (d, J=8.61 Hz, 2H), 7.31 (d, J=8.61 Hz, 2H), 2.75 (d, J=5.09 Hz, 3H).

Step 3: To a solution of Pd(dppf)Cl₂ (24.0 mg, 0.033 mmol) in dioxane (3 mL) were added bis(pinacolato)diboron (150 mg, 0.591 mmol), 5-bromo-N-methyl-6-(4-(trifluoromethyl)phenoxy)pyridine-3-sulfonamide (135 mg, 0.328 mmol) and potassium acetate (97 mg, 0.99 mmol). The reaction mixture was stirred at 80° C. for 1 h under microwave irradiation. The reaction mixture was cooled to room temperature and concentrated to afford crude (5-(N-methylsulfamoyl)-2-(4-(trifluoromethyl) phenoxy) pyridin-3-yl) boronic acid. MS (ESI) m/z C₁₃H₁₂BF₃N₂O₅S [M+1]⁺ calc'd 377, found 377.

### Intermediate 1-24. Preparation of 4-(4-(difluoromethyl)phenoxy)-N-methyl-3-(4,4,5,5-tetramethyl-1,3,2-dioxaborolan-2-yl)benzenesulfonamide

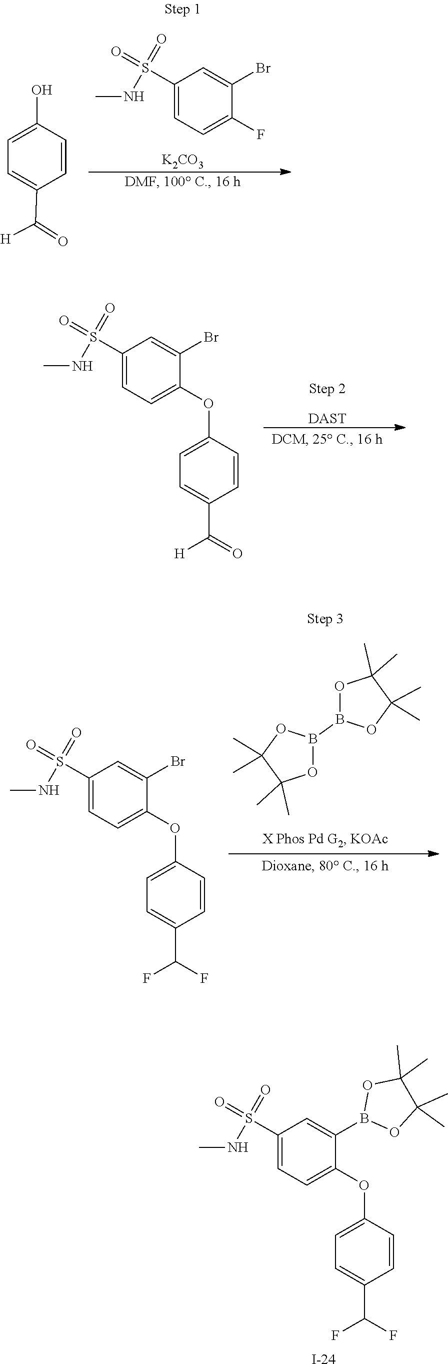

Step 1: K₂CO₃ (3.40 g, 24.6 mmol) was added to a stirred mixture of 4-hydroxybenzaldehyde (1 g, 8.19 mmol) in DMF (40 mL) at RT, and the mixture was stirred at RT for 10 min. 3-Bromo-4-fluoro-N-methylbenzenesulfonamide (2.20 g, 8.19 mmol) was added to the solution, and the reaction mixture was heated with stirring to 90° C. for 16 h. The reaction was quenched with H₂O (100 mL), and the organic layer was extracted with EtOAc (40 mL×3). The combined organic layers were washed with brine (10 mL), dried over anhydrous Na₂SO₄, filtered and concentrated under reduced pressure. The residue was purified by flash silica gel chromatography (0-30% EtOAc in petroleum ether) to afford 3-bromo-4-(4-formylphenoxy)-N-methylbenzenesulfonamide. MS (ESI) m/z C₁₄H₁₃BrNO₄S [M+1]⁺ calc'd 370, 372, found 370, 372. ¹H NMR (500 MHz, CDCl₃) δ 10.15 (s, 1H), 8.36 (d, J=2.1 Hz, 1H), 8.12-8.08 (m, 2H), 7.98 (dd, J=2.3, 8.5 Hz, 1H), 7.43 (s, 1H), 7.32-7.28 (m, 2H), 2.91 (d, J=5.5 Hz, 3H). Step 2: To a stirred solution of 3-bromo-4-(4-formylphenoxy)-N-methylbenzenesulfonamide (370 mg, 0.999 mmol) in DCM (5 mL) was added DAST (0.858 mL, 6.50 mmol) at 25° C. After the addition was finished, the reaction was stirred at 25° C. for 16 h. The reaction was quenched carefully with sat. Na₂CO₃ (10 mL). The mixture was extracted with ethyl acetate (10 mL×3).

The combined organic layers were washed with brine (10 mL), dried over anhydrous Na₂SO₄, filtered and concentrated under reduced pressure. The residue was purified by flash silica gel chromatography (0-5% EtOAc in petroleum ether) to afford 3-bromo-4-(4-(difluoromethyl)phenoxy)-N-methylbenzenesulfonamide. MS (ESI) m/z C₁₄H₁₃BrF₂NO₃S [M+1]⁺ calc'd 392, 394, found 392, 394.

Step 3: A mixture of 3-bromo-4-(4-(difluoromethyl)phenoxy)-N-methylbenzenesulfonamide (45 mg, 0.115 mmol), KOAc (24.8 mg, 0.252 mmol), PdCl₂(dppf) (8.4 mg, 0.011 mmol) and bis(pinacolato)diboron (58.3 mg, 0.229 mmol) in dioxane (7 mL) was degassed and backfilled with N₂ (three times). The mixture was heated to 80° C. for 16 h. The mixture was cooled to room temperature, filtered, and the filtrate was concentrated under reduced pressure to afford crude 4-(4-(difluoromethyl)phenoxy)-N-methyl-3-(4,4,5,5-tetramethyl-1,3,2-dioxaborolan-2-yl)benzenesulfonamide.

### SYNTHESIS OF EXAMPLE COMPOUNDS

### Example 1-1

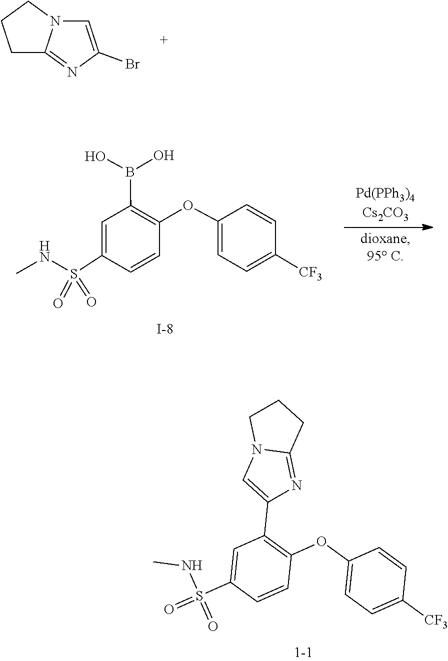

3-(6,7-Dihydro-5H-pyrrolo[1,2-α]imidazol-2-yl)-N-methyl-4-(4-(trifluoromethyl)phenoxy) benzenesulfonamide

(5-(N-Methylsulfamoyl)-2-(4-(trifluoromethyl)phenoxy)phenyl)boronic acid (I-8, 200 mg, 0.53 mmol), 2-bromo-6,7-dihydro-5H-pyrrolo[1,2-a]imidazole (199 mg, 1.07 mmol), Cs₂CO₃ (695 mg, 2.13 mmol), and Pd(PPh₃)₄ (31 mg, 0.027 mmol) were combined. The mixture was diluted in dioxane (5.33 mL), and the reaction vessel was purged with N₂ for 3 min. The vial was sealed and heated to 95° C. for 60 min. The reaction was cooled to RT, filtered through Celite with EtOAc, and concentrated under reduced pressure. The residue was purified by flash silica gel chromatography (3:1 ethyl acetate/EtOH in hexanes, 15-60%) to afford 3-(6,7-dihydro-5H-pyrrolo[1,2-α]imidazol-2-yl)-N-methyl-4-(4-(trifluoromethyl)phenoxy) benzenesulfonamide. MS (ESI) m/z C₂₀H₁₉F₃N₃O₃S [M+1]⁺ calc'd 438, found 438. ¹H NMR (499 MHz, DMSO-d₆) δ 8.62 (d, J=2.4 Hz, 1H), 7.79 (d, J=8.7 Hz, 2H), 7.60 (dd, J=8.5, 2.4 Hz, 1H), 7.54-7.46 (m, 2H), 7.23 (dd, J=19.5, 8.6 Hz, 3H), 3.96 (t, J=7.1 Hz, 2H), 2.78 (t, J=7.5 Hz, 2H), 2.55-2.49 (m, 2H), 2.45 (d, J=4.5 Hz, 3H).

### Example 1-2

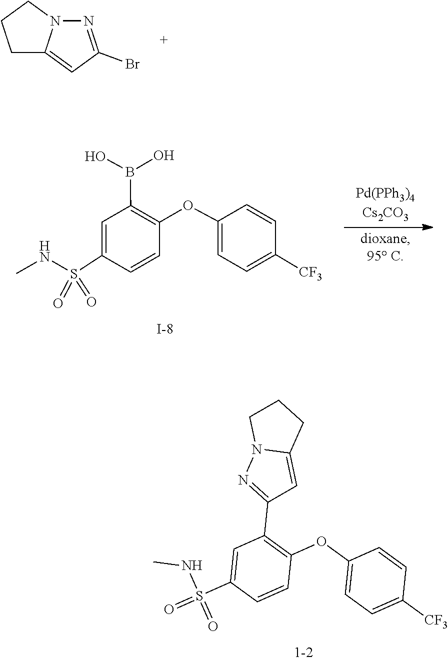

3-(5,6-Dihydro-4H-pyrrolo[1,2-b]pyrazol-2-yl)-N-methyl-4-(4-(trifluoromethyl)phenoxy) benzenesulfonamide

(5-(N-Methylsulfamoyl)-2-(4-(trifluoromethyl)phenoxy)phenyl)boronic acid (I-8, 65 mg, 0.17 mmol), 2-bromo-5,6-dihydro-4H-pyrrolo[1,2-b]pyrazole hydrochloride (77 mg, 0.35 mmol), Cs₂CO₃ (226 mg, 0.69 mmol) and Pd(PPh₃)₄ (20 mg, 0.017 mmol) were combined. The mixture was diluted in dioxane (1.90 mL) and water (271 μl), and the reaction vessel was purged with N₂ for 3 min. The mixture was sealed and heated to 95° C. for 60 min. The mixture was cooled to RT, diluted in MeOH, and filtered. The filtrate was purified by preparative HPLC (reverse phase, C₁₈, ACN/water with 0.05% NH₃ modifier), to afford 3-(5,6-dihydro-4H-pyrrolo[1,2-b]pyrazol-2-yl)-N-methyl-4-(4-(trifluoromethyl)phenoxy)benzenesulfonamide. MS (ESI) m/z C₂₀H₁₉F₃N₃O₃S [M+1]⁺ calc'd 438, found 438. ¹H NMR (499 MHz, DMSO-d₆) δ 8.48 (d, J=2.4 Hz, 1H), 7.77 (d, J=8.7 Hz, 2H), 7.71 (dd, J=8.5, 2.4 Hz, 1H), 7.56 (s, 1H), 7.29 (d, J=8.5 Hz, 1H), 7.21 (d, J=8.6 Hz, 2H), 6.39 (s, 1H), 4.17-4.05 (m, 2H), 2.83 (t, J=7.3 Hz, 2H), 2.54 (d, J=7.1 Hz, 2H), 2.45 (d, J=4.5 Hz, 3H).

### Example 1-3

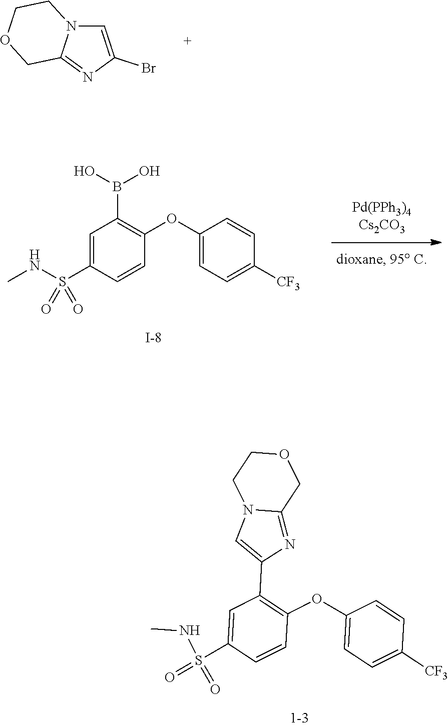

3-(5,6-Dihydro-8H-imidazo[2,1-c][1,4]oxazin-2-yl)-N-methyl-4-(4-(trifluoromethyl)phenoxy) benzenesulfonamide

(5-(N-Methylsulfamoyl)-2-(4-(trifluoromethyl)phenoxy)phenyl)boronic acid (I-8, 50 mg, 0.13 mmol), 2-bromo-5,6-dihydro-8H-imidazo[2,1-c][1,4]oxazine (41 mg, 0.20 mmol), Cs₂CO₃ (130 mg, 0.40 mmol) and Pd(PPh₃)₄ (15 mg, 0.013 mmol) were combined. The mixture was diluted with dioxane (1.46 mL) and water (208 μL), and the reaction mixture was purged with N₂ for 3 min. The mixture was sealed and heated to 90° C. for 60 min. The reaction mixture was cooled to RT, diluted in MeOH, filtered, and concentrated under reduced pressure. The residue was purified by preparative HPLC (reverse phase, C₁₈, ACN/water with 0.1% TFA modifier) to afford 3-(5,6-dihydro-8H-imidazo[2,1-c] [1,4]oxazin-2-yl)-N-methyl-4-(4-(trifluoromethyl)phenoxy) benzenesulfonamide, TFA. MS (ESI) m/z C₂₀H₁₉F₃N₃O₄S [M+1]⁺ calc'd 454, found 454. ¹H NMR (499 MHz, DMSO-d₆) δ 8.60 (d, J=2.4 Hz, 1H), 7.81 (d, J=8.7 Hz, 2H), 7.62 (dd, J=8.5, 2.4 Hz, 1H), 7.58 (s, 1H), 7.51 (d, J=4.0 Hz, 1H), 7.29 (d, J=8.6 Hz, 2H), 7.19 (d, J=8.5 Hz, 1H), 4.81 (s, 2H), 4.09-3.95 (m, 4H), 2.44 (d, J=3.3 Hz, 3H).

### Example 1-4

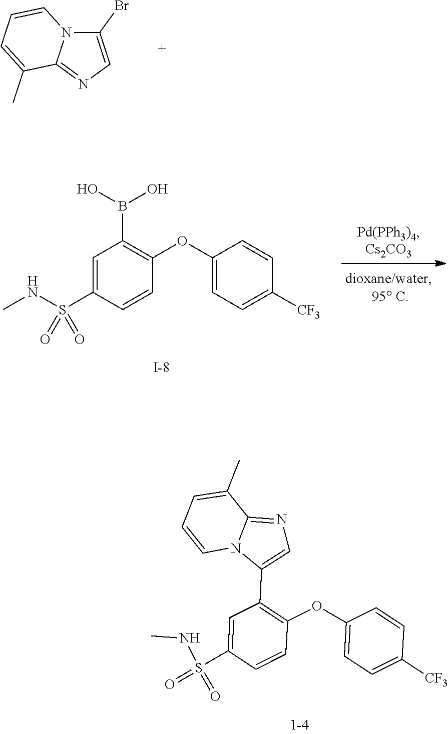

N-Methyl-3-(8-methylimidazo[1,2-a]pyridin-3-yl)-4-(4-(trifluoromethyl) phenoxy)benzenesulfonamide

To (5-(N-Methylsulfamoyl)-2-(4-(trifluoromethyl)phenoxy)phenyl)boronic acid (I-8, 647 mg for 23 reactions, 1 equiv.) in a stock vial was added dioxane (17.5 mL) and Pd(PPh₃)₄ (199 mg, 0.1 equiv) to a total volume of 18.2 mL. In a separate vial was added Cs₂CO₃ (2248 mg for 23 reactions, 4 equiv.) and water (3.04 mL). To pre-dosed reaction vials containing 3-bromo-8-methylimidazo[1,2-a]pyridine (0.15 mmol, 2 equiv.) were added the aforementioned cesium carbonate solution (0.22 mL) followed by the boronic acid stock solution (0.75 mL) at RT. Each reaction vessel was purged with a needle of N₂ for 5 min, and heated to 95° C. for 16 h. The mixture was cooled, diluted with DMSO, filtered and purified by preparative HPLC (reverse phase, C₁₈, ACN/water with 0.1% TFA modifier) to afford N-methyl-3-(8-methylimidazo[1,2-a]pyridin-3-yl)-4-(4-(trifluoromethyl) phenoxy)benzenesulfonamide. MS (ESI) m/z C₂₂H₁₉F3N₃O₃S [M+1]⁺ calc'd 462, found 462. ¹H NMR (499 MHz, DMSO-d₆) δ 1H NMR (500 MHz, DMSO-d₆) δ 8.47 (d, J=6.0 Hz, 1H), 8.29 (s, 1H), 8.04 (s, 1H), 7.97 (d, J=8.8 Hz, 1H), 7.77 (d, J=8.5 Hz, 2H), 7.68-7.53 (m, 2H), 7.32 (dd, J=14.9, 8.5 Hz, 3H), 7.26 (s, 1H), 2.59 (s, 3H), 2.55 (s, 3H).

Compounds in Table 3 below were prepared from common Intermediates 1-8, 1-24 or from Table 2 using the methods described in Example 1-1-1-4.

TABLE 3

| | | | Exact |
|---|---|---|---|
| | | | Mass |
| Ex. No. | Structure | IUPAC Name | [M + H]+ |
| 1-5 | 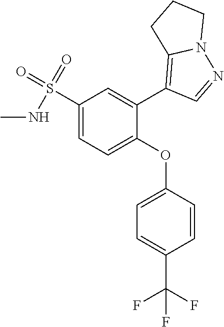 [SMILES: CNS(=O)(=O)c1ccc(Oc2ccc(C(F)(F)F)cc2)c(-c2cnn3c2CCC3)c1] | 3-(5,6-dihydro-4H- pyrrolo[1,2-b]pyrazol- 3-yl)-N-methyl-4-[4- (trifluoromethyl)phenoxy] benzene-1-sulfonamide | Calc'd 438, found 438 |
| 1-6 | 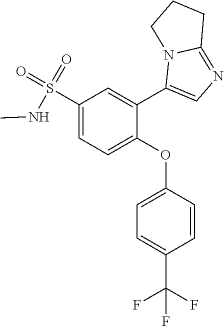 [SMILES: CNS(=O)(=O)c1ccc(Oc2ccc(C(F)(F)F)cc2)c(-c2cnc3n2CCC3)c1] | 3-(6,7-dihydro-5H- pyrrolo[1,2-a]imidazol- 3-yl)-N-methyl-4-[4- (trifluoromethyl)phenoxy] benzene-1-sulfonamide | Calc'd 438, found 438 |
| 1-7 | 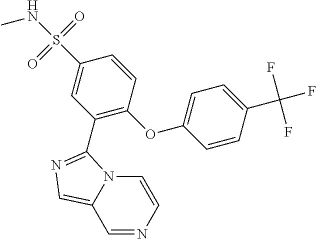 [SMILES: CNS(=O)(=O)c1ccc(Oc2ccc(C(F)(F)F)cc2)c(-c2ncc3cnccn23)c1] | 3-(imidazo[1,5- a]pyrazin-3-yl)-N- methyl-4-[4- (trifluoromethyl)phenoxy] benzene-1-sulfonamide | Calc'd 449, found 449 |
| 1-8 | 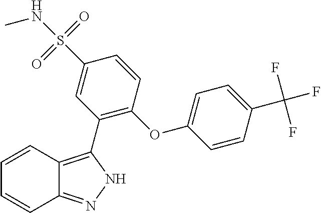 [SMILES: CNS(=O)(=O)c1ccc(Oc2ccc(C(F)(F)F)cc2)c(-c2[nH]nc3ccccc23)c1] | 3-(2H-indazol-3-yl)-N- methyl-4-[4- (trifluoromethyl)phenoxy] benzene-1-sulfonamide | Calc'd 448, found 448 |
| 1-9 | 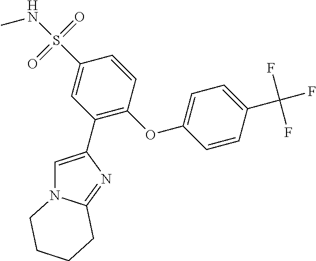 [SMILES: CNS(=O)(=O)c1ccc(Oc2ccc(C(F)(F)F)cc2)c(-c2cn3c(n2)CCCC3)c1] | N-methyl-3-(5,6,7,8- tetrahydroimidazo[1,2- a]pyridin-2-yl)-4-[4- (trifluoromethyl)phenoxy] benzene-1-sulfonamide | Calc'd 452, found 452 |
| 1-10 | 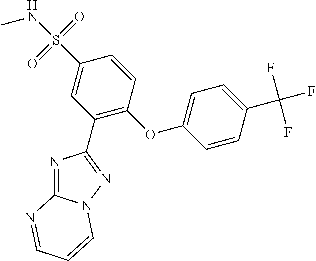 [SMILES: CNS(=O)(=O)c1ccc(Oc2ccc(C(F)(F)F)cc2)c(-c2nc3ncccn3n2)c1] | N-methyl-3- ([1,2,4]triazolo[1,5- a]pyrimidin-2-yl)-4-[4- (trifluoromethyl)phenoxy] benzene-1-sulfonamide | Calc'd 450, found 450 |
| 1-11 | 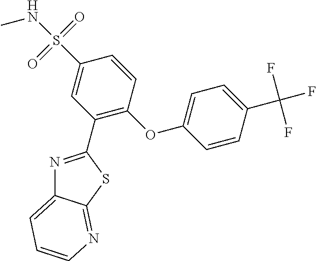 [SMILES: CNS(=O)(=O)c1ccc(Oc2ccc(C(F)(F)F)cc2)c(-c2nc3cccnc3s2)c1] | N-methyl-3- ([1,3]thiazolo[5,4- b]pyridin-2-yl)-4-[4- (trifluoromethyl)phenoxy] benzene-1-sulfonamide | Calc'd 466, found 466 |
| 1-12 | 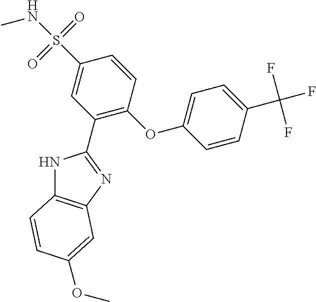 [SMILES: CNS(=O)(=O)c1ccc(Oc2ccc(C(F)(F)F)cc2)c(-c2nc3cc(OC)ccc3[nH]2)c1] | 3-(5-methoxy-1H- benzimidazol-2-yl)-N- methyl-4-[4- (trifluoromethyl)phenoxy] benzene-1-sulfonamide | Calc'd 478, found 478 |
| 1-13 | 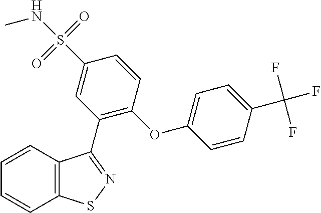 [SMILES: CNS(=O)(=O)c1ccc(Oc2ccc(C(F)(F)F)cc2)c(-c2nsc3ccccc23)c1] | 3-(1,2-benzothiazol-3- yl)-N-methyl-4-[4- (trifluoromethyl)phenoxy] benzene-1-sulfonamide | Calc'd 465, found 465 |
| 1-14 | 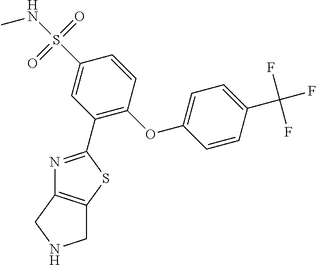 [SMILES: CNS(=O)(=O)c1ccc(Oc2ccc(C(F)(F)F)cc2)c(-c2nc3c(s2)CNC3)c1] | 3-(5,6-dihydro-4H- pyrrolo[3,4- d][1,3]thiazol-2-yl)-N- methyl-4-[4- (trifluoromethyl)phenoxy] benzene-1-sulfonamide | Calc'd 456, found 456 |
| 1-15 | 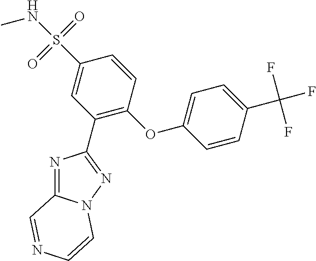 [SMILES: CNS(=O)(=O)c1ccc(Oc2ccc(C(F)(F)F)cc2)c(-c2nc3cnccn3n2)c1] | N-methyl-3- ([1,2,4]triazolo[1,5- a]pyrazin-2-yl)-4-[4- (trifluoromethyl)phenoxy] benzene-1-sulfonamide | Calc'd 450, found 450 |
| 1-16 | 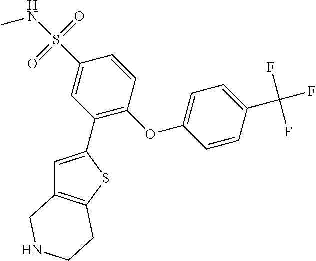 [SMILES: CNS(=O)(=O)c1ccc(Oc2ccc(C(F)(F)F)cc2)c(-c2cc3c(s2)CCNC3)c1] | N-methyl-3-(4,5,6,7- tetrahydrothieno[3,2- c]pyridin-2-yl)-4-[4- (trifluoromethyl)phenoxy] benzene-1-sulfonamide | Calc'd 469, found 469 |
| 1-17 | 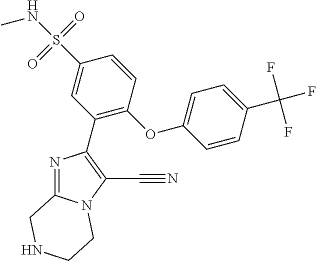 [SMILES: CNS(=O)(=O)c1ccc(Oc2ccc(C(F)(F)F)cc2)c(-c2nc3n(c2C#N)CCNC3)c1] | 3-(3-cyano-5,6,7,8- tetrahydroimidazo[1,2- a]pyrazin-2-yl)-N- methyl-4-[4- (trifluoromethyl)phenoxy] benzene-1-sulfonamide | Calc'd 478, found 478 |
| 1-18 | 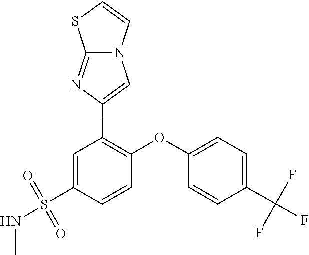 [SMILES: CNS(=O)(=O)c1ccc(Oc2ccc(C(F)(F)F)cc2)c(-c2cn3ccsc3n2)c1] | 3-(imidazo[2,1-b] [1,3]thiazol-6-yl)-N- methyl-4-[4- (trifluoromethyl)phenoxy] benzene-1-sulfonamide | Calc'd 454, found 454 |
| 1-19 | 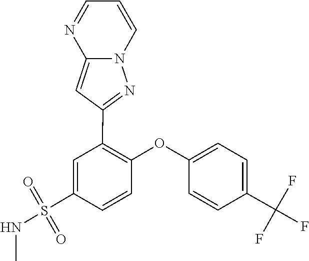 [SMILES: CNS(=O)(=O)c1ccc(Oc2ccc(C(F)(F)F)cc2)c(-c2cc3ncccn3n2)c1] | N-methyl-3- (pyrazolo[1,5-a] pyrimidin-2-yl)-4-[4- (trifluoromethyl)phenoxy] benzene-1-sulfonamide | Calc'd 449, found 449 |
| 1-20 | 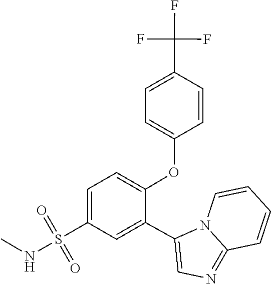 [SMILES: CNS(=O)(=O)c1ccc(Oc2ccc(C(F)(F)F)cc2)c(-c2cnc3ccccn23)c1] | 3-(imidazo[1,2- a]pyridin-3-yl)-N- methyl-4-[4- (trifluoromethyl)phenoxy] benzene-1-sulfonamide | Calc'd 448, found 448 |
| 1-21 | 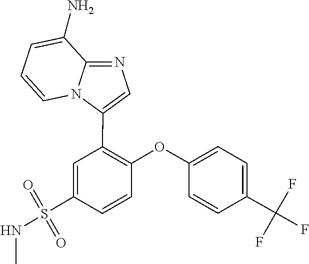 [SMILES: CNS(=O)(=O)c1ccc(Oc2ccc(C(F)(F)F)cc2)c(-c2cnc3c(N)cccn23)c1] | 3-(8- aminoimidazo[1,2- a]pyridin-3-yl)-N- methyl-4-[4- (trifluoromethyl)phenoxy] benzene-1-sulfonamide | Calc'd 463, found 463 |
| 1-22 | 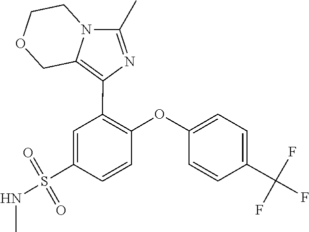 [SMILES: CNS(=O)(=O)c1ccc(Oc2ccc(C(F)(F)F)cc2)c(-c2nc(C)n3c2COCC3)c1] | N-methyl-3-(3-methyl- 5,6-dihydro-8H- imidazo[5,1-c] [1,4]oxazin-1-yl)-4-[4- (trifluoromethyl)phenoxy] benzene-1-sulfonamide | Calc'd 468, found 468 |
| 1-23 | 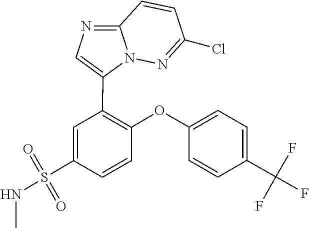 [SMILES: CNS(=O)(=O)c1ccc(Oc2ccc(C(F)(F)F)cc2)c(-c2cnc3ccc(Cl)nn23)c1] | 3-(6- chloroimidazo[1,2- b]pyridazin-3-yl)-N- methyl-4-[4- (trifluoromethyl)phenoxy] benzene-1-sulfonamide | Calc'd 483, found 483 |
| 1-24 | 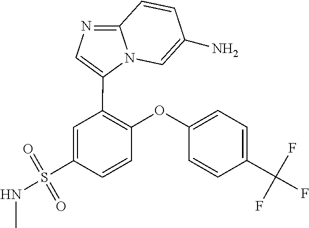 [SMILES: CNS(=O)(=O)c1ccc(Oc2ccc(C(F)(F)F)cc2)c(-c2cnc3ccc(N)cn23)c1] | 3-(6- aminoimidazo[1,2- a]pyridin-3-yl)-N- methyl-4-[4- (trifluoromethyl)phenoxy] benzene-1-sulfonamide | Calc'd 463, found 463 |
| 1-25 | 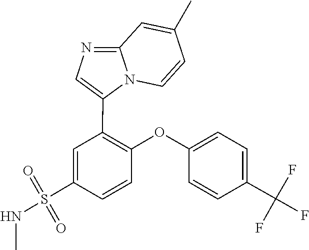 [SMILES: CNS(=O)(=O)c1ccc(Oc2ccc(C(F)(F)F)cc2)c(-c2cnc3cc(C)ccn23)c1] | N-methyl-3-(7- methylimidazo[1,2- a]pyridin-3-yl)-4-[4- (trifluoromethyl)phenoxy] benzene-1-sulfonamide | Calc'd 462, found 462 |
| 1-26 | 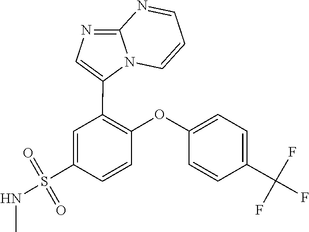 [SMILES: CNS(=O)(=O)c1ccc(Oc2ccc(C(F)(F)F)cc2)c(-c2cnc3ncccn23)c1] | 3-(imidazo[1,2- a]pyrimidin-3-yl)-N- methyl-4-[4- (trifluoromethyl)phenoxy] benzene-1-sulfonamide | Calc'd 449, found 449 |
| 1-27 | 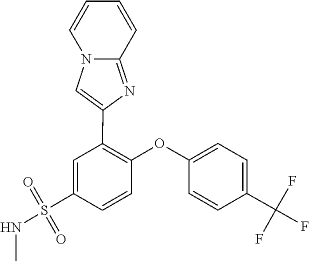 [SMILES: CNS(=O)(=O)c1ccc(Oc2ccc(C(F)(F)F)cc2)c(-c2cn3ccccc3n2)c1] | 3-(imidazo[1,2- a]pyridin-2-yl)-N- methyl-4-[4- (trifluoromethyl)phenoxy] benzene-1-sulfonamide | Calc'd 448, found 448 |
| 1-28 | 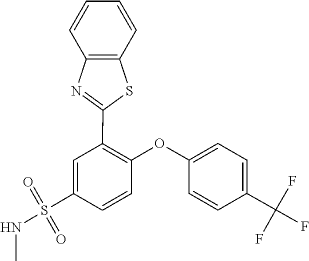 [SMILES: CNS(=O)(=O)c1ccc(Oc2ccc(C(F)(F)F)cc2)c(-c2nc3ccccc3s2)c1] | 3-(1,3-benzothiazol-2- yl)-N-methyl-4-[4- (trifluoromethyl)phenoxy] benzene-1-sulfonamide | Calc'd 465, found 465 |
| 1-29 | 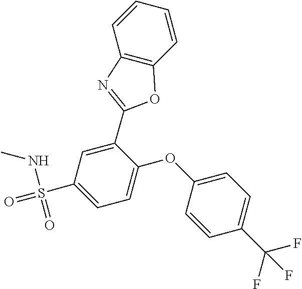 [SMILES: CNS(=O)(=O)c1ccc(Oc2ccc(C(F)(F)F)cc2)c(-c2nc3ccccc3o2)c1] | 3-(1,3-benzoxazol-2- yl)-N-methyl-4-[4- (trifluoromethyl)phenoxy] benzene-1-sulfonamide | Calc'd 449, found 449 |
| 1-30 | 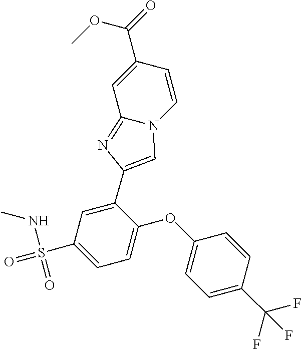 [SMILES: CNS(=O)(=O)c1ccc(Oc2ccc(C(F)(F)F)cc2)c(-c2cn3ccc(C(=O)OC)cc3n2)c1] | methyl 2-{5- (methylsulfamoyl)-2-[4- (trifluoromethyl)phenoxy] phenyl}imidazo[1,2-a] pyridine-7-carboxylate | Calc'd 506, found 506 |
| 1-31 | 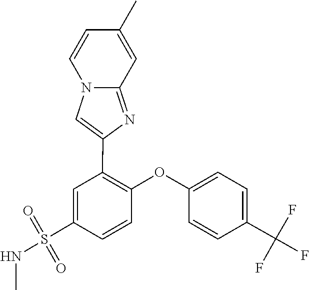 [SMILES: CNS(=O)(=O)c1ccc(Oc2ccc(C(F)(F)F)cc2)c(-c2cn3ccc(C)cc3n2)c1] | N-methyl-3-(7- methylimidazo[1,2- a]pyridin-2-yl)-4-[4- (trifluoromethyl)phenoxy] benzene-1-sulfonamide | Calc'd 462, found 462 |
| 1-32 | 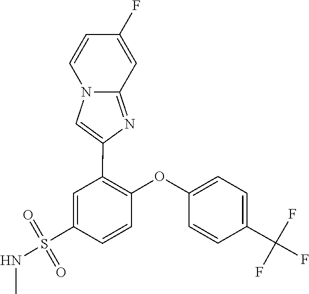 [SMILES: CNS(=O)(=O)c1ccc(Oc2ccc(C(F)(F)F)cc2)c(-c2cn3ccc(F)cc3n2)c1] | 3-(7-fluoroimidazo[1,2- a]pyridin-2-yl)-N- methyl-4-[4- (trifluoromethyl)phenoxy] benzene-1-sulfonamide | Calc'd 466, found 466 |
| 1-33 | 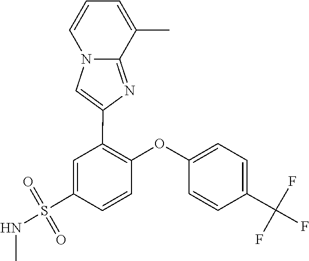 [SMILES: CNS(=O)(=O)c1ccc(Oc2ccc(C(F)(F)F)cc2)c(-c2cn3cccc(C)c3n2)c1] | N-methyl-3-(8- methylimidazo[1,2- a]pyridin-2-yl)-4-[4- (trifluoromethyl)phenoxy] benzene-1-sulfonamide | Calc'd 462, found 462 |
| 1-34 | 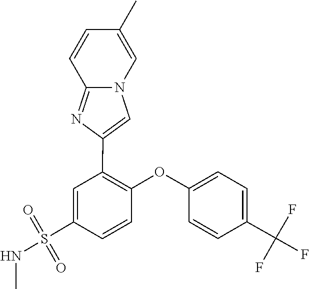 [SMILES: CNS(=O)(=O)c1ccc(Oc2ccc(C(F)(F)F)cc2)c(-c2cn3cc(C)ccc3n2)c1] | N-methyl-3-(6- methylimidazo[1,2- a]pyridin-2-yl)-4-[4- (trifluoromethyl)phenoxy] benzene-1-sulfonamide | Calc'd 462, found 462 |
| 1-35 | 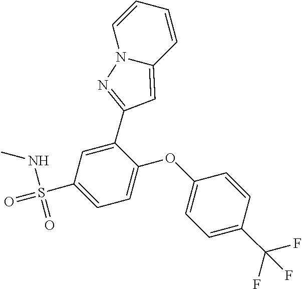 [SMILES: CNS(=O)(=O)c1ccc(Oc2ccc(C(F)(F)F)cc2)c(-c2cc3ccccn3n2)c1] | N-methyl-3- (pyrazolo[1,5- a]pyridin-2-yl)-4-[4- (trifluoromethyl)phenoxy] benzene-1-sulfonamide | Calc'd 448, found 448 |
| 1-36 | 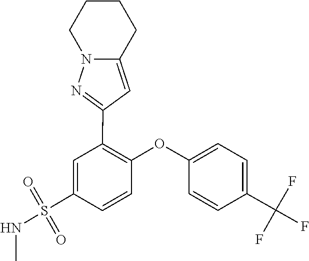 [SMILES: CNS(=O)(=O)c1ccc(Oc2ccc(C(F)(F)F)cc2)c(-c2cc3n(n2)CCCC3)c1] | N-methyl-3-(4,5,6,7- tetrahydropyrazolo[1,5- a]pyridin-2-yl)-4-[4- (trifluoromethyl)phenoxy] benzene-1-sulfonamide | Calc'd 452, found 452 |
| 1-37 | 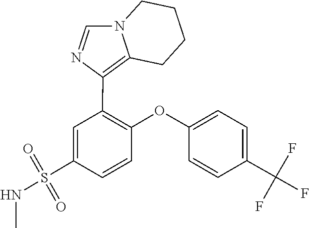 [SMILES: CNS(=O)(=O)c1ccc(Oc2ccc(C(F)(F)F)cc2)c(-c2ncn3c2CCCC3)c1] | N-methyl-3-(5,6,7,8- tetrahydroimidazo[1,5- a]pyridin-1-yl)-4-[4- (trifluoromethyl)phenoxy] benzene-1-sulfonamide | Calc'd 452, found 452 |
| 1-38 | 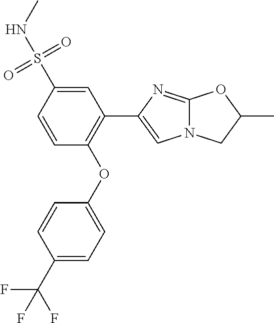 [SMILES: CNS(=O)(=O)c1ccc(Oc2ccc(C(F)(F)F)cc2)c(-c2cn3c(n2)OC(C)C3)c1] | (R)- or (S)-N-methyl- 3-(2-methyl-2,3- dihydroimidazo[2,1-b] [1,3]oxazol-6-yl)-4-[4- (trifluoromethyl)phenoxy] benzene-1-sulfonamide | Calc'd 454, found 454 |
| 1-39 | 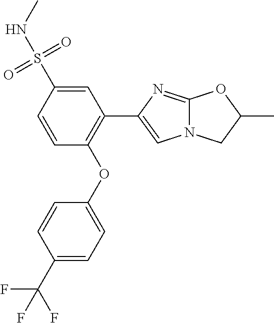 [SMILES: CNS(=O)(=O)c1ccc(Oc2ccc(C(F)(F)F)cc2)c(-c2cn3c(n2)OC(C)C3)c1] | (S)- or (R)-N-methyl-3- (2-methyl-2,3- dihydroimidazo[2,1- b][1,3]oxazol-6-yl)-4-[4- (trifluoromethyl)phenoxy] benzene-1-sulfonamide | Calc'd 454, found 454 |
| 1-40 | 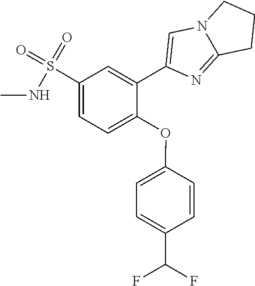 [SMILES: CNS(=O)(=O)c1ccc(Oc2ccc(C(F)F)cc2)c(-c2cn3c(n2)CCC3)c1] | 4-[4- (difluoromethyl)phenoxy]- 3-(6,7-dihydro-5H- pyrrolo[1,2-a]imidazol- 2-yl)-N- methylbenzene-1- sulfonamide | Calc'd 420, found 420 |
| 1-41 | 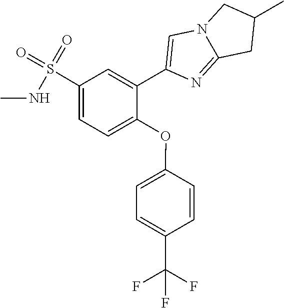 | (R)- or (S)-N-methyl-3- (6-methyl-6,7-dihydro- 5H-pyrrolo[1,2- a]imidazol-2-yl)-4-[4- (trifluoromethyl)phenoxy] benzene-1-sulfonamide | Calc'd 452, found 452 |
| 1-42 | 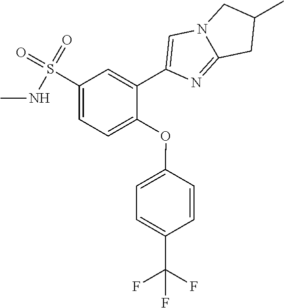 | (S)- or (R)-N-methyl-3- (6-methyl-6,7-dihydro- 5H-pyrrolo[1,2- a]imidazol-2-yl)-4-[4- (trifluoromethyl)phenoxy] benzene-1-sulfonamide | Calc'd 452, found 452 |
| 1-43 | 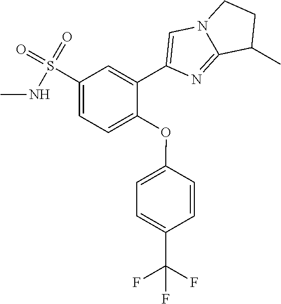 | (R)- or (S)-N-methyl-3- (7-methyl-6,7-dihydro- 5H-pyrrolo[1,2- a]imidazol-2-yl)-4-[4- (trifluoromethyl)phenoxy] benzene-1-sulfonamide | Calc'd 452, found 452 |
| 1-44 | 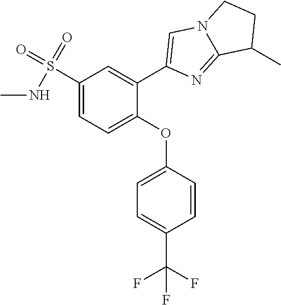 | (S)- or (R)-N-methyl-3- (7-methyl-6,7-dihydro- 5H-pyrrolo[1,2- a]imidazol-2-yl)-4-[4- (trifluoromethyl)phenoxy] benzene-1-sulfonamide | Calc'd 452, found 452 |
| 1-45 | 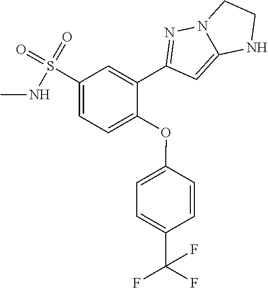 [SMILES: CNS(=O)(=O)c1ccc(Oc2ccc(C(F)(F)F)cc2)c(-c2cc3n(n2)CCN3)c1] | 3-(2,3-dihydro-1H- imidazo[1,2-b]pyrazol- 6-yl)-N-methyl-4-[4- (trifluoromethyl)phenoxy] benzene-1-sulfonamide | Calc'd 439, found 439 |
| 1-46 | 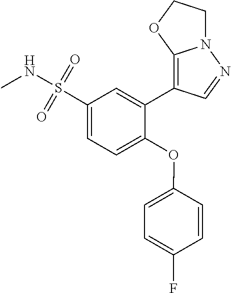 [SMILES: CNS(=O)(=O)c1ccc(Oc2ccc(F)cc2)c(-c2cnn3c2OCC3)c1] | 3-(2,3- dihydropyrazolo[5,1- b]oxazol-7-yl)-4-(4- fluorophenoxy)-N- methylbenzenesulfonamide | Calc'd 390, found 390 |
| 1-47 | 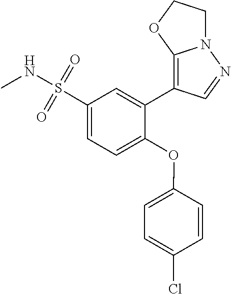 [SMILES: CNS(=O)(=O)c1ccc(Oc2ccc(Cl)cc2)c(-c2cnn3c2OCC3)c1] | 4-(4-chlorophenoxy)-3- (2,3- dihydropyrazolo[5,1- b]oxazol-7-yl)-N- methylbenzenesulfonamide | Calc'd 406, found 406 |
| 1-48 | 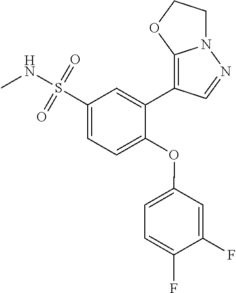 [SMILES: CNS(=O)(=O)c1ccc(Oc2ccc(F)c(F)c2)c(-c2cnn3c2OCC3)c1] | 4-(3,4- difluorophenoxy)-3- (2,3- dihydropyrazolo[5,1- b]oxazol-7-yl)-N- methylbenzenesulfonamide | Calc'd 408, found 408 |
| 1-49 | 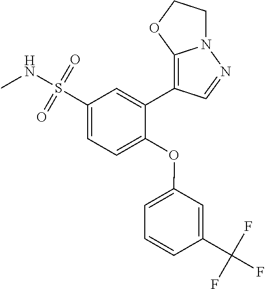 [SMILES: CNS(=O)(=O)c1ccc(Oc2cccc(C(F)(F)F)c2)c(-c2cnn3c2OCC3)c1] | 3-(2,3- dihydropyrazolo[5,1- b]oxazol-7-yl)-N- methyl-4-(3- (trifluoromethyl)phenoxy) benzenesulfonamide | Calc'd 440, found 440 |
| 1-50 | 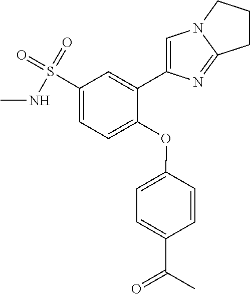 [SMILES: CNS(=O)(=O)c1ccc(Oc2ccc(C(C)=O)cc2)c(-c2cn3c(n2)CCC3)c1] | 4-(4-acetylphenoxy)-3- (6,7-dihydro-5H- pyrrolo[1,2-a]imidazol- 2-yl)-N- methylbenzene-1- sulfonamide | Calc'd 412, found 412 |

### Example 2

### Example 2-1

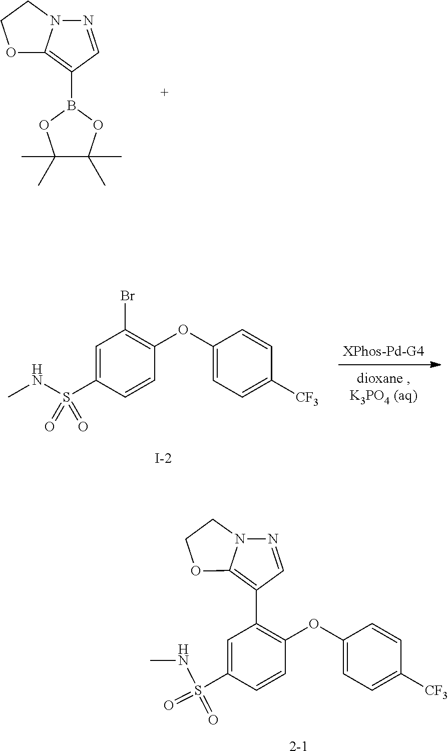

3-(2,3-Dihydropyrazolo[5,1-b]oxazol-7-yl)-N-methyl-4-(4-(trifluoromethyl) phenoxy) benzenesulfonamide

A 24 mL stock solution of dioxane containing 3-bromo-N-methyl-4-(4-(trifluoromethyl)phenoxy)benzenesulfonamide (I-2, 985 mg, 2.4 mmol, 0.1 M) and XPhos-Pd-G4 (207 mg, 0.24 mmol, 0.01 M) was prepared. To 7-(4,4,5,5-tetramethyl-1,3,2-dioxaborolan-2-yl)-2,3-dihydropyrazolo[5,1-b]oxazole (26 mg, 0.11 mmol) was added 1 mL of the aforementioned stock solution (0.1 mmol I-2, 0.01 mmol XPhos-Pd-G4). To the resulting solution was added K₃PO₄ (2 M in water, 0.1 mL, 0.2 mmol), and the resulting biphasic mixture was heated to 100° C. overnight, then cooled to RT. The reaction mixture was stirred with MgSO₄, diluted with DMSO, and then filtered. The crude product solution was purified by preparative HPLC (reverse phase, C₁₈, ACN/water with 0.1% TFA modifier) to afford 3-(2,3-dihydropyrazolo[5,1-b]oxazol-7-yl)-N-methyl-4-(4-(trifluoromethyl)phenoxy) benzenesulfonamide. MS (ESI) m/z C₁₉H₁₇F3N₃O₄S [M+1]‘ calc’d 440, found 440. ¹H NMR (500 MHz, DMSO-d₆) δ 8.03 (d, J=2.3 Hz, 1H), 7.75 (d, J=8.7 Hz, 2H), 7.69 (s, 1H), 7.59 (dd, J=8.5, 2.3 Hz, 1H), 7.52 (q, J=4.9 Hz, 1H), 7.27 (d, J=8.5 Hz, 1H), 7.18 (d, J=8.6 Hz, 2H), 5.24 (t, J=8.1 Hz, 2H), 4.31 (t, J=8.1 Hz, 2H), 2.45 (d, J=5.0 Hz, 3H).

### Example 2-2

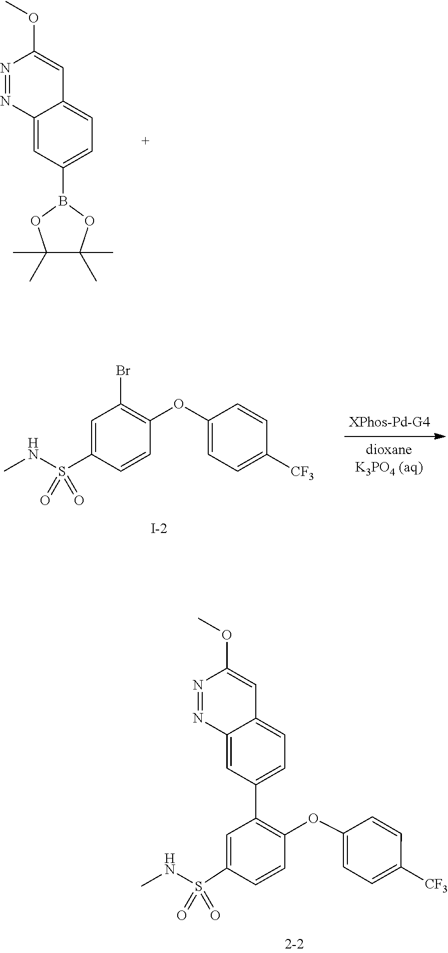

3-(3-Methoxycinnolin-7-yl)-N-methyl-4-[4-(trifluoromethyl)phenoxy]benzene-1-sulfonamide,

A 24 mL stock solution of dioxane containing 3-bromo-N-methyl-4-(4-(trifluoromethyl)phenoxy)benzenesulfonamide (I-2, 985 mg, 2.4 mmol, 0.1 M) and XPhos-Pd-G4 (207 mg, 0.24 mmol, 0.01 M) was prepared. To 3-methoxy-7-(4,4,5,5-tetramethyl-1,3,2-dioxaborolan-2-yl)cinnoline (32 mg, 0.11 mmol) was added 1 mL of the aforementioned stock solution (0.1 mmol I-2, 0.01 mmol XPhos-Pd-G4). To the resulting solution was added K₃PO₄ (2 M in water, 0.1 mL, 0.2 mmol), and the resulting biphasic mixture was heated to 100° C. overnight, then cooled to RT. The reaction mixture was stirred with MgSO₄, diluted with DMSO, and then filtered. The crude product solution was purified by preparative HPLC (reverse phase, C₁₈, ACN/water with 0.1% TFA modifier) to afford 3-(3-methoxycinnolin-7-yl)-N-methyl-4-[4-(trifluoromethyl)phenoxy]benzene-1-sulfonamide. MS (ESI) m/z C₂₃H₁₉F₃N₃O₄S [M+1]⁺ calc'd 490, found 490. ¹H NMR (500 MHz, DMSO-d₆) δ 8.49 (s, 1H), 8.07 (d, J=2.0 Hz, 1H), 7.98 (q, J=8.8 Hz, 2H), 7.88 (dd, J=8.6, 2.0 Hz, 1H), 7.74 (d, J=8.6 Hz, 2H), 7.71 (s, 1H), 7.56 (t, J=4.9 Hz, 1H), 7.33 (d, J=8.6 Hz, 1H), 7.30 (d, J=8.4 Hz, 2H), 4.17 (s, 3H), 2.54 (s, 3H).

Compounds in Table 4 below were prepared from common Intermediate 1-2 or 1-24 bromide using the method described in Example 2-1.

TABLE 4

| | | | Exact |
|---|---|---|---|
| | | | Mass |
| Example | Structure | IUPAC Name | [M + H]+ |
| 2-3 | 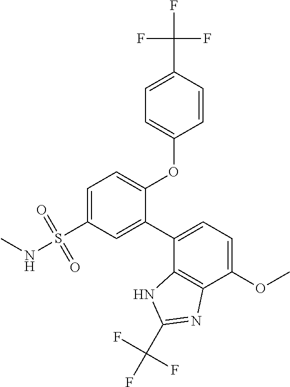 | 3-[4-methoxy-2- (trifluoromethyl)- 1H- benzimidazol-7- yl]-N-methyl-4-[4- (trifluoromethyl) phenoxy]benzene- 1-sulfonamide | Calc'd 546, found 546 |
| 2-4 | 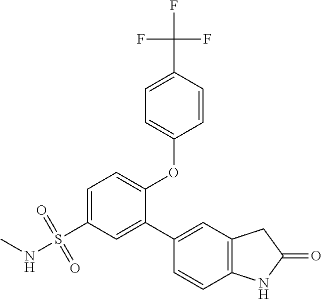 [SMILES: CNS(=O)(=O)c1ccc(Oc2ccc(C(F)(F)F)cc2)c(-c2ccc3c(c2)CC(=O)N3)c1] | N-methyl-3-(2- oxo-2,3-dihydro- 1H-indol-5-yl)- 4-[4- (trifluoromethyl) phenoxy]benzene- 1-sulfonamide | Calc'd 463, found 463 |
| 2-5 | 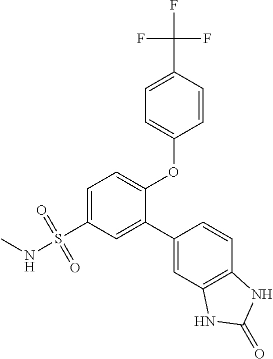 [SMILES: CNS(=O)(=O)c1ccc(Oc2ccc(C(F)(F)F)cc2)c(-c2ccc3[nH]c(=O)[nH]c3c2)c1] | N-methyl-3-(2- oxo-2,3-dihydro- 1H- benzimidazol-5- yl)-4-[4- (trifluoromethyl) phenoxy]benzene- 1-sulfonamide | Calc'd 464, found 464 |
| 2-6 | 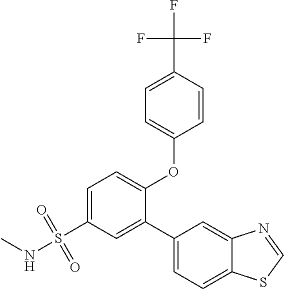 [SMILES: CNS(=O)(=O)c1ccc(Oc2ccc(C(F)(F)F)cc2)c(-c2ccc3scnc3c2)c1] | 3-(1,3- benzothiazol-5- yl)-N-methyl-4-[4- (trifluoromethyl) phenoxy]benzene- 1-sulfonamide | Calc'd 465, found 465 |
| 2-7 | 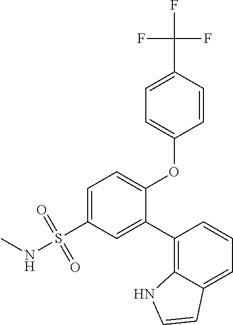 [SMILES: CNS(=O)(=O)c1ccc(Oc2ccc(C(F)(F)F)cc2)c(-c2cccc3cc[nH]c23)c1] | 3-(1H-indol-7- yl)-N-methyl-4-[4- (trifluoromethyl) phenoxy]benzene- 1-sulfonamide | Calc'd 447, found 447 |
| 2-8 | 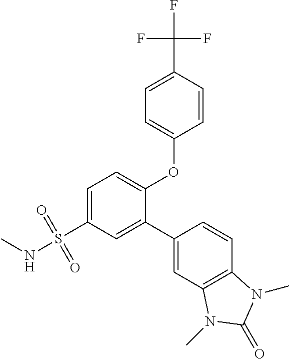 | 3-(1,3-dimethyl- 2-oxo-2,3- dihydro-1H- benzimidazol-5- yl)-N-methyl-4-[4- (trifluoromethyl) phenoxy]benzene- 1-sulfonamide | Calc'd 492, found 492 |
| 2-9 | 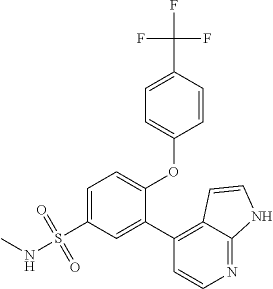 [SMILES: CNS(=O)(=O)c1ccc(Oc2ccc(C(F)(F)F)cc2)c(-c2ccnc3[nH]ccc23)c1] | N-methyl-3-(1H- pyrrolo[2,3- b]pyridin-4-yl)- 4-[4- (trifluoromethyl) phenoxy]benzene- 1-sulfonamide | Calc'd 448, found 448 |
| 2-10 | 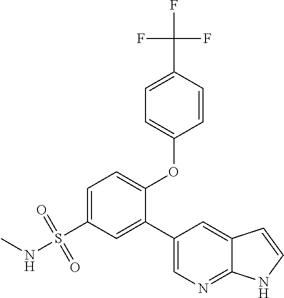 [SMILES: CNS(=O)(=O)c1ccc(Oc2ccc(C(F)(F)F)cc2)c(-c2cnc3[nH]ccc3c2)c1] | N-methyl-3-(1H- pyrrolo[2,3- b]pyridin-5-yl)- 4-[4- (trifluoromethyl) phenoxy]benzene- 1-sulfonamide | Calc'd 448, found 448 |
| 2-11 | 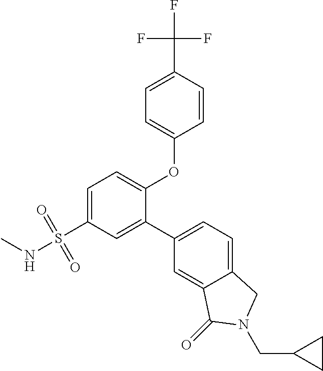 [SMILES: CNS(=O)(=O)c1ccc(Oc2ccc(C(F)(F)F)cc2)c(-c2ccc3c(c2)C(=O)N(CC2CC2)C3)c1] | 3-[2- (cyclopropylmethyl)- 3-oxo-2,3- dihydro-1H- isoindol-5-yl]-N- methyl-4-[4- (trifluoromethyl) phenoxy]benzene- 1-sulfonamide | Calc'd 517, found 517 |
| 2-12 | 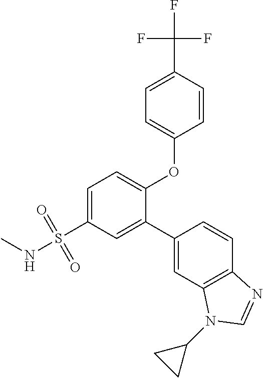 [SMILES: CNS(=O)(=O)c1ccc(Oc2ccc(C(F)(F)F)cc2)c(-c2ccc3ncn(C4CC4)c3c2)c1] | 3-(1- cyclopropyl-1H- benzimidazol-6- yl)-N-methyl-4-[4- (trifluoromethyl) phenoxy]benzene- 1-sulfonamide | Calc'd 488, found 488 |
| 2-13 | 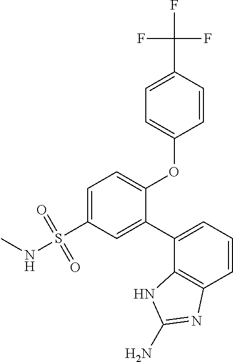 [SMILES: CNS(=O)(=O)c1ccc(Oc2ccc(C(F)(F)F)cc2)c(-c2cccc3nc(N)[nH]c23)c1] | 3-(2-amino-1H- benzimidazol-7- yl)-N-methyl-4-[4- (trifluoromethyl) phenoxy]benzene- 1-sulfonamide | Calc'd 463, found 463 |
| 2-14 | 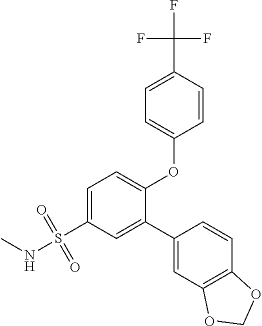 [SMILES: CNS(=O)(=O)c1ccc(Oc2ccc(C(F)(F)F)cc2)c(-c2ccc3c(c2)OCO3)c1] | 3-(2H-1,3- benzodioxol-5- yl)-N-methyl-4-[4- (trifluoromethyl) phenoxy]benzene- 1-sulfonamide | Calc'd 452, found 452 |
| 2-15 | 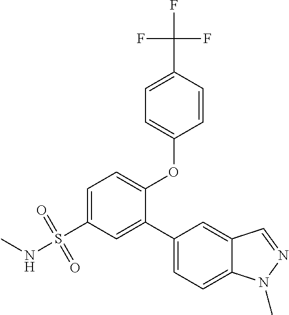 [SMILES: CNS(=O)(=O)c1ccc(Oc2ccc(C(F)(F)F)cc2)c(-c2ccc3c(cnn3C)c2)c1] | N-methyl-3-(1- methyl-1H- indazol-5-yl)-4-[4- (trifluoromethyl) phenoxy]benzene- 1-sulfonamide | Calc'd 462, found 462 |
| 2-16 | 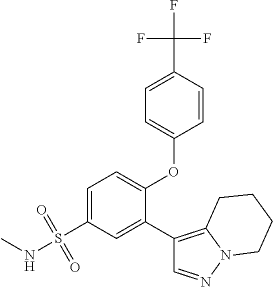 [SMILES: CNS(=O)(=O)c1ccc(Oc2ccc(C(F)(F)F)cc2)c(-c2cnn3c2CCCC3)c1] | N-methyl-3- (4,5,6,7- tetrahydropyrazolo [1,5-a]pyridin- 3-yl)-4-[4- (trifluoromethyl) phenoxy]benzene- 1-sulfonamide | Calc'd 452, found 452 |
| 2-17 | 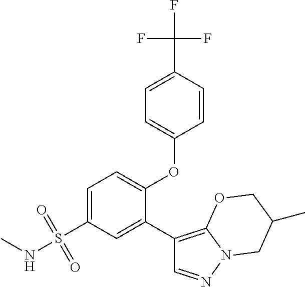 | N-methyl-3-(6- methyl-6,7- dihydro-5H- pyrazolo[5,1- b][1,3]oxazin-3- yl)-4-[4- (trifluoromethyl) phenoxy]benzene- 1-sulfonamide | Calc'd 468, found 468 |
| 2-18 | 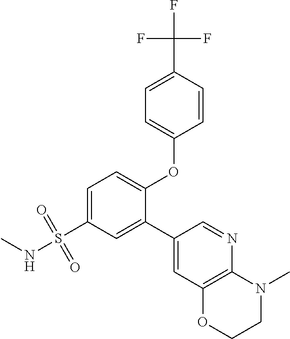 | N-methyl-3-(4- methyl-3,4- dihydro-2H- pyrido[3,2- b][1,4]oxazin-7- yl)-4-[4- (trifluoromethyl) phenoxy]benzene- 1-sulfonamide | Calc'd 480, found 480 |
| 2-19 | 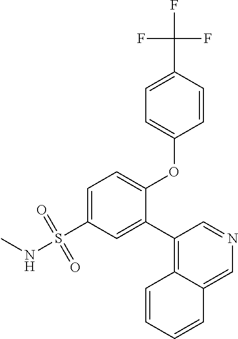 [SMILES: CNS(=O)(=O)c1ccc(Oc2ccc(C(F)(F)F)cc2)c(-c2cncc3ccccc23)c1] | 3-(isoquinolin-4- yl)-N-methyl-4-[4- (trifluoromethyl) phenoxy]benzene- 1-sulfonamide | Calc'd 459, found 459 |
| 2-20 | 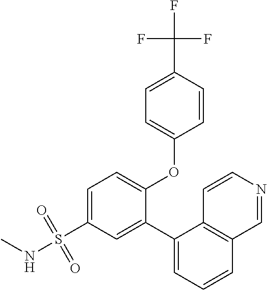 [SMILES: CNS(=O)(=O)c1ccc(Oc2ccc(C(F)(F)F)cc2)c(-c2cccc3cnccc23)c1] | 3-(isoquinolin-5- yl)-N-methyl-4-[4- (trifluoromethyl) phenoxy]benzene- 1-sulfonamide | Calc'd 459, found 459 |
| 2-21 | 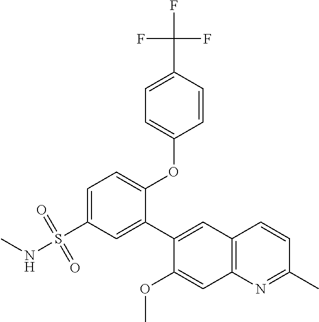 | 3-(7-methoxy-2- methylquinolin- 6-yl)-N-methyl-4-[4- (trifluoromethyl) phenoxy]benzene- 1-sulfonamide | Calc'd 503, found 503 |
| 2-22 | 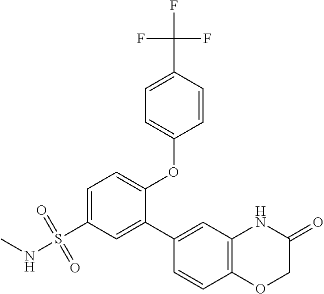 [SMILES: CNS(=O)(=O)c1ccc(Oc2ccc(C(F)(F)F)cc2)c(-c2ccc3c(c2)NC(=O)CO3)c1] | N-methyl-3-(3- oxo-3,4-dihydro- 2H-1,4- benzoxazin-6- yl)-4-[4- (trifluoromethyl) phenoxy]benzene- 1-sulfonamide | Calc'd 479, found 479 |
| 2-23 | 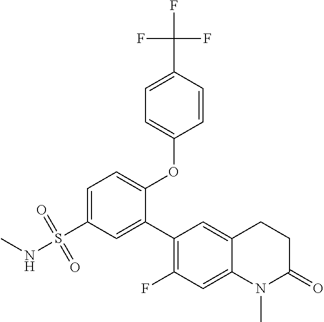 [SMILES: CNS(=O)(=O)c1ccc(Oc2ccc(C(F)(F)F)cc2)c(-c2cc3c(cc2F)N(C)C(=O)CC3)c1] | 3-(7-fluoro-1- methyl-2-oxo- 1,2,3,4- tetrahydroquinolin- 6-yl)-N- methyl-4-[4- (trifluoromethyl) phenoxy]benzene- 1-sulfonamide | Calc'd 509, found 509 |
| 2-24 | 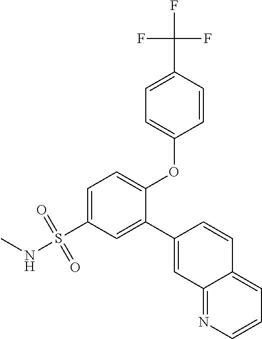 [SMILES: CNS(=O)(=O)c1ccc(Oc2ccc(C(F)(F)F)cc2)c(-c2ccc3cccnc3c2)c1] | N-methyl-3- (quinolin-7-yl)- 4-[4- (trifluoromethyl) phenoxy]benzene- 1-sulfonamide | Calc'd 459, found 459 |
| 2-25 | 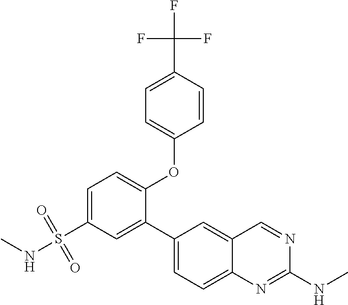 | N-methyl-3-[2- (methylamino) quinazolin-6-yl]- 4-[4- (trifluoromethyl) phenoxy]benzene- 1-sulfonamide | Calc'd 489, found 489 |
| 2-26 | 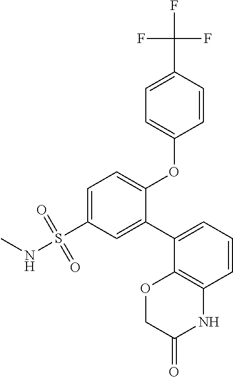 [SMILES: CNS(=O)(=O)c1ccc(Oc2ccc(C(F)(F)F)cc2)c(-c2cccc3c2OCC(=O)N3)c1] | N-methyl-3-(3- oxo-3,4-dihydro- 2H-1,4- benzoxazin-8- yl)-4-[4- (trifluoromethyl) phenoxy]benzene- 1-sulfonamide | Calc'd 479, found 479 |
| 2-27 | 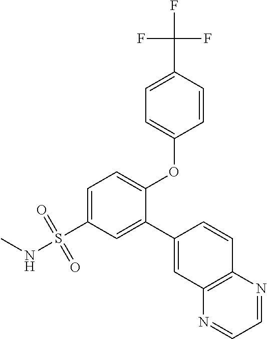 [SMILES: CNS(=O)(=O)c1ccc(Oc2ccc(C(F)(F)F)cc2)c(-c2ccc3nccnc3c2)c1] | N-methyl-3- (quinoxalin-6- yl)-4-[4- (trifluoromethyl) phenoxy]benzene- 1-sulfonamide | Calc'd 460, found 460 |
| 2-28 | 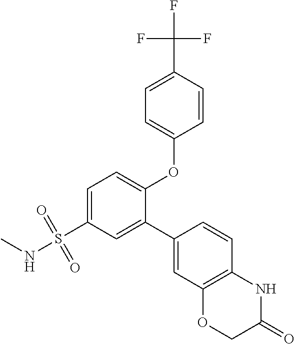 [SMILES: CNS(=O)(=O)c1ccc(Oc2ccc(C(F)(F)F)cc2)c(-c2ccc3c(c2)OCC(=O)N3)c1] | N-methyl-3-(3- oxo-3,4-dihydro- 2H-1,4- benzoxazin-7- yl)-4-[4- (trifluoromethyl) phenoxy]benzene- 1-sulfonamide | Calc'd 479, found 479 |
| 2-29 | 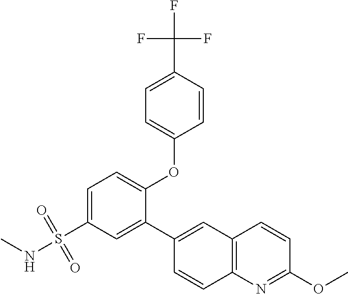 | 3-(2- methoxyquinolin- 6-yl)-N-methyl- 4-[4- (trifluoromethyl) phenoxy]benzene- 1-sulfonamide | Calc'd 489, found 489 |
| 2-30 | 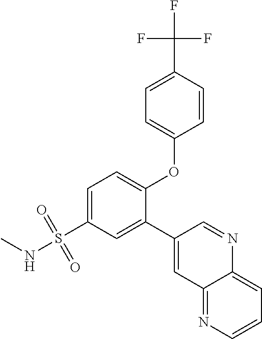 [SMILES: CNS(=O)(=O)c1ccc(Oc2ccc(C(F)(F)F)cc2)c(-c2cnc3cccnc3c2)c1] | N-methyl-3-(1,5- naphthyridin-3- yl)-4-[4- (trifluoromethyl) phenoxy]benzene- 1-sulfonamide | Calc'd 460, found 466 |
| 2-31 | 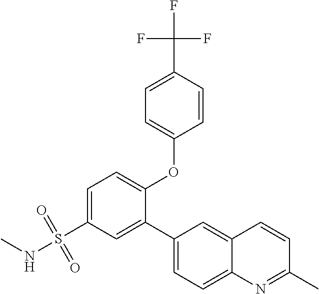 | N-methyl-3-(2- methylquinolin- 6-yl)-4-[4- (trifluoromethyl) phenoxy]benzene- 1-sulfonamide | Calc'd 473, found 473 |
| 2-32 | 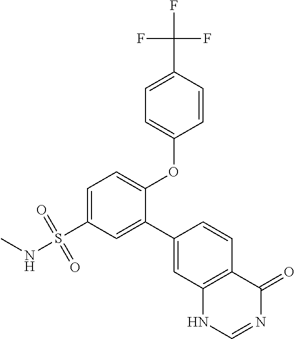 [SMILES: CNS(=O)(=O)c1ccc(Oc2ccc(C(F)(F)F)cc2)c(-c2ccc3c(=O)nc[nH]c3c2)c1] | N-methyl-3-(4- oxo-1,4- dihydroquinazolin- 7-yl)-4-[4- (trifluoromethyl) phenoxy]benzene- 1-sulfonamide | Calc'd 476, found 476 |
| 2-33 | 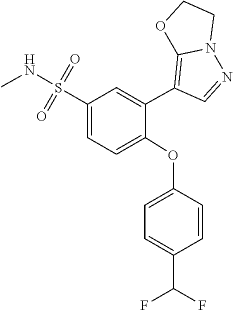 [SMILES: CNS(=O)(=O)c1ccc(Oc2ccc(C(F)F)cc2)c(-c2cnn3c2OCC3)c1] | (difluoromethyl) phenoxy]-3-(2,3- dihydropyrazolo[5,1- b][1,3]oxazol-7- yl)-N- methylbenzene- 1-sulfonamide | Calc'd 422, found 422 |

### Example 3-1

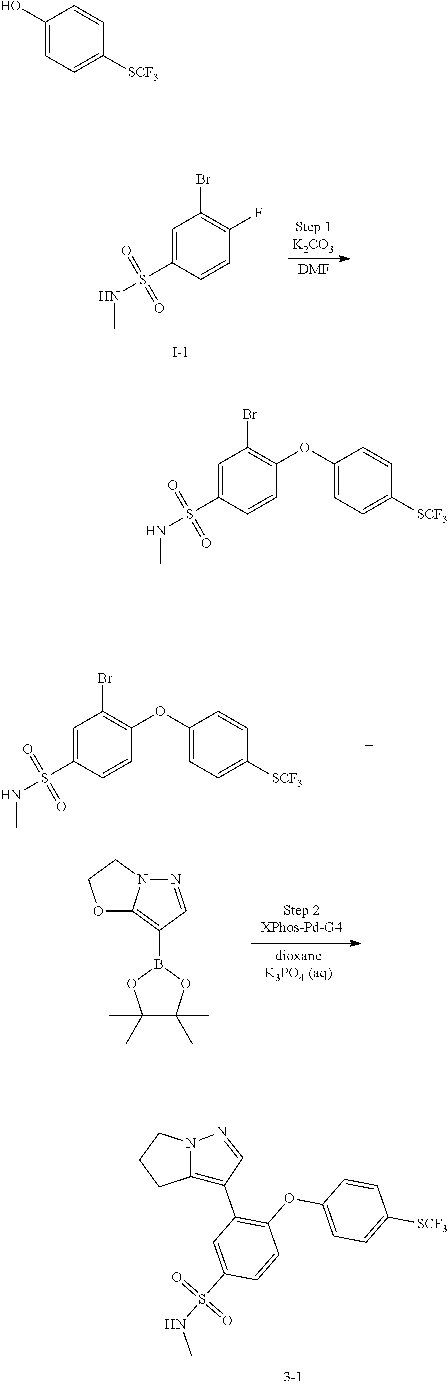

3-(5,6-Dihydro-4H-pyrrolo[1,2-b]pyrazol-3-yl)-N-methyl-4-(4-((trifluoromethyl) thio)phenoxy)benzenesulfonamide

Step 1. A 16 mL stock solution of DMF containing 3-bromo-4-fluoro-N-methylbenzenesulfonamide (I-1, 644 mg, 2.4 mmol, 0.15 M) was prepared. To 4-((trifluoromethyl)thio)phenol (36 mg, 0.11 mmol) was added 0.67 mL of the aforementioned stock solution (0.1 mmol I-1). To the resulting solution was added K₂CO₃ (42 mg, 0.3 mmol). The resulting suspension was heated to 100° C. in a microwave reactor for 90 min. The reaction mixture was filtered and used as a crude solution in DMF in the next reaction. MS (ESI) m/z C₁₄H₁₂BrF₃NO₃S₂ [M+1]⁺ calc'd 442, found 442.

Step 2. A 6 mL stock solution of DMF containing XPhos-Pd-G4 (207 mg, 0.24 mmol, 0.04 M) was prepared. A 6 mL stock solution of DMF containing 7-(4,4,5,5-tetramethyl-1,3,2-dioxaborolan-2-yl)-2,3-dihydropyrazolo[5,1-b]oxazole (618 mg, 2.64 mmol, 0.44 M) was prepared. To 0.25 mL of the aforementioned XPhos-Pd-G4 stock solution (0.01 mmol XPhos-Pd-G4) were added the crude solution from Step 1 and 0.25 mL of the aforementioned 7-(4,4,5,5-tetramethyl-1,3,2-dioxaborolan-2-yl)-2,3-dihydropyrazolo[5,1-b]oxazole stock solution (0.11 mmol 7-(4,4,5,5-tetramethyl-1,3,2-dioxaborolan-2-yl)-2,3-dihydropyrazolo[5,1-b]oxazole). To the resulting solution was added aqueous K₃PO₄ (2 M in water, 0.15 mL, 0.3 mmol) and the resulting biphasic mixture was heated to 100° C. overnight, then cooled to RT. The reaction mixture was stirred with MgSO₄, diluted with DMSO, and then filtered. The crude product solution was purified by preparative HPLC (reverse phase, C₁₈, ACN/water with 0.1% TFA modifier) to afford 3-(5,6-dihydro-4H-pyrrolo[1,2-b]pyrazol-3-yl)-N-methyl-4-(4-((trifluoromethyl)thio)phenoxy) benzenesulfonamide. MS (ESI) m/z C₂₀H₁₉F₃N₃O₃S2 [M+1]+ calc'd 470, found 470. ¹H NMR (500 MHz, DMSO-d₆) δ 7.90 (s, 1H), 7.78 (s, 1H), 7.73 (d, J=8.4 Hz, 2H), 7.66-7.61 (m, 1H), 7.52 (q, J=4.8 Hz, 1H), 7.23 (d, J=8.5 Hz, 1H), 7.17 (d, J=8.6 Hz, 2H), 4.08 (t, J=7.2 Hz, 2H), 3.02 (t, J=7.3 Hz, 2H), 2.57 (q, J=7.3 Hz, 2H), 2.54 (s, 3H).

Compounds in Table 5 below were prepared from common Intermediate I-1 using the method described in Example 3-1.

TABLE 5

| | | | Exact |
|---|---|---|---|
| | | | Mass |
| Example | Structure | IUPAC Name | [M + H]+ |
| 3-2 | 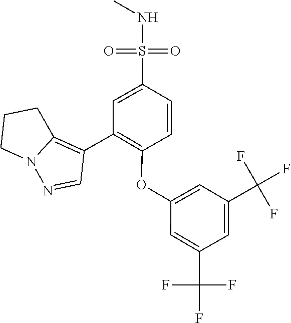 [SMILES: CNS(=O)(=O)c1ccc(Oc2cc(C(F)(F)F)cc(C(F)(F)F)c2)c(-c2cnn3c2CCC3)c1] | 4-[3,5- bis(trifluoromethyl) phenoxy]-3-(5,6-dihydro-4H- pyrrolo[1,2-b]pyrazol-3- yl)-N-methylbenzene-1- sulfonamide | Calc'd 506, found 506 |
| 3-3 | 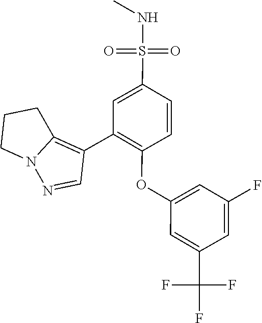 [SMILES: CNS(=O)(=O)c1ccc(Oc2cc(F)cc(C(F)(F)F)c2)c(-c2cnn3c2CCC3)c1] | 3-(5,6-dihydro-4H- pyrrolo[1,2-b]pyrazol-3- yl)-4-[3-fluoro-5- (trifluoromethyl)phenoxy]- N-methylbenzene-1- sulfonamide | Calc'd 456, found 456 |
| 3-4 | 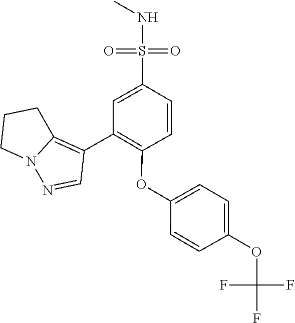 [SMILES: CNS(=O)(=O)c1ccc(Oc2ccc(OC(F)(F)F)cc2)c(-c2cnn3c2CCC3)c1] | 3-(5,6-dihydro-4H- pyrrolo[1,2-b]pyrazol-3- yl)-N-methyl-4-[4- (trifluoromethoxy)phenoxy] benzene-1-sulfonamide | Calc'd 454, found 454 |
| 3-5 | 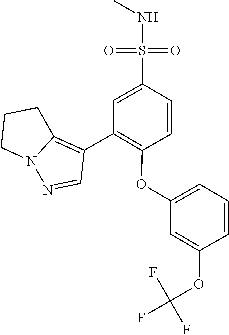 [SMILES: CNS(=O)(=O)c1ccc(Oc2cccc(OC(F)(F)F)c2)c(-c2cnn3c2CCC3)c1] | 3-(5,6-dihydro-4H- pyrrolo[1,2-b]pyrazol-3- yl)-N-methyl-4-[3- (trifluoromethoxy)phenoxy] benzene-1-sulfonamide | Calc'd 454, found 454 |
| 3-6 | 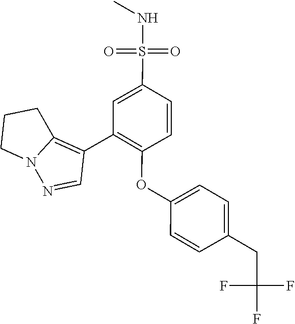 [SMILES: CNS(=O)(=O)c1ccc(Oc2ccc(CC(F)(F)F)cc2)c(-c2cnn3c2CCC3)c1] | 3-(5,6-dihydro-4H- pyrrolo[1,2-b]pyrazol-3- yl)-N-methyl-4-[4-(2,2,2- trifluoroethyl)phenoxy] benzene-1-sulfonamide | Calc'd 452, found 452 |

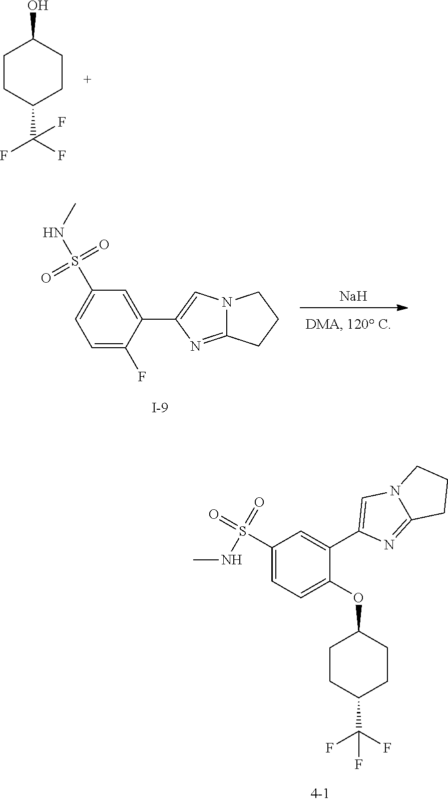

3-(6,7-Dihydro-5H-pyrrolo[1,2-α]imidazol-2-yl)-N-methyl-4-(((1r,4r)-4-(trifluoromethyl) cyclohexyl)oxy) benzenesulfonamide

To sodium hydride (60 wt % in mineral oil) (17 mg, 0.42 mmol) in DMA (0.5 mL) was added trans-4-(trifluoromethyl)cyclohexan-1-ol (85 mg, 0.51 mmol), and the mixture was stirred at RT for 30 min. 3-(6,7-Dihydro-5H-pyrrolo[1,2-α]imidazol-2-yl)-4-fluoro-N-methylbenzene sulfonamide (I-9, 25 mg, 0.085 mmol) in dry DMA (212 μl) was added in one portion and the mixture was heated to 120° C. overnight. The mixture was cooled to RT, diluted in DMA/MeOH, and filtered. The crude product solution was purified by preparative HPLC (reverse phase, C18, ACN/water with 0.05% NH₃ modifier) to afford 3-(6,7-dihydro-5H-pyrrolo[1,2-α]imidazol-2-yl)-N-methyl-4-(((1R,4R)-4-(trifluoromethyl)cyclohexyl)oxy) benzenesulfonamide. MS (ESI) m z C₂₀H₂₅F₃N₃O₃S [M+1]⁺ calc'd 444, found 444. ¹H NMR (499 MHz, DMSO-d₆) δ 8.49 (s, 1H), 7.56 (s, 1H), 7.50 (s, 1H), 7.33 (d, J=11.7 Hz, 2H), 4.62 (s, 1H), 4.01 (s, 2H), 2.78 (s, 2H), 2.57-2.52 (m, 3H), 2.38 (s, 3H), 2.24 (s, 2H), 1.96 (s, 2H), 1.65-1.43 (m, 4H).

### Example 4-2

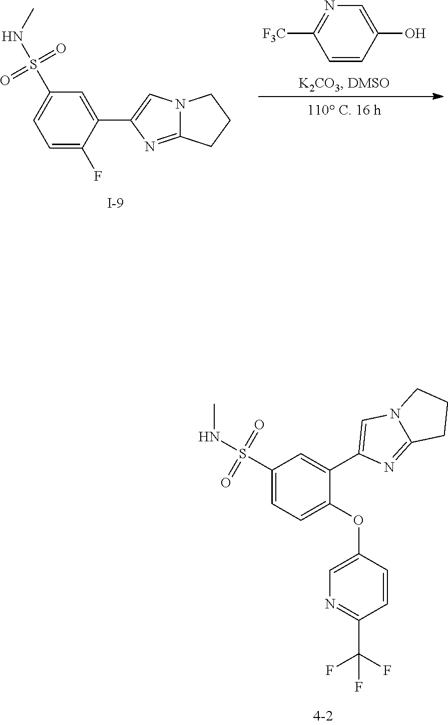

3-(6,7-dihydro-5H-pyrrolo[1,2-α]imidazol-2-yl)-N-methyl-4-((6-(trifluoromethyl)pyridin-3-yl)oxy)benzenesulfonamide

To a solution of 3-(6,7-dihydro-5H-pyrrolo[1,2-α]imidazol-2-yl)-4-fluoro-N-methylbenzenesulfonamide (100 mg, 0.339 mmol)) and 6-(trifluoromethyl)pyridin-3-ol (138 mg, 0.847 mmol) in DMSO (3 ml) was added K₂CO₃ (234 mg, 1.69 mmol) under N₂. The reaction mixture was stirred at 120° C. for 16 h. The residue was cooled to room temperature and purified by preparative HPLC (reverse phase C-18 column), eluting with Acetonitrile/Water+0.1% TFA to give 3-(6,7-dihydro-5H-pyrrolo[1,2-α]imidazol-2-yl)-N-methyl-4-((6-(trifluoromethyl)pyridin-3-yl)oxy)benzenesulfonamide as a solid. MS (ESI) m/z C₁₉H₁₈F₃N₄O₃S [M+H]⁺ calc'd 439, found 439. ¹H NMR (400 MHz, METHANOL-d₄) δ ppm 2.60 (s, 3H) 2.74-2.85 (m, 2H) 3.23 (br t, J=7.43 Hz, 2H) 4.29 (br t, J=7.04 Hz, 2H) 7.28 (d, J=8.61 Hz, 1H) 7.75 (br d, J=8.61 Hz, 1H) 7.89 (dt, J=8.61, 2.35 Hz, 2H) 7.96 (s, 1H) 8.30 (d, J=1.56 Hz, 1H) 8.61 (s, 1H).

Compounds in Table 6 below were prepared from common Intermediate I-9 using the method described in Example 4-2.

TABLE 6

| | | | Exact |
|---|---|---|---|
| | | | Mass |
| Example | Structure | IUPAC Name | [M + H]+ |
| 4-3 | 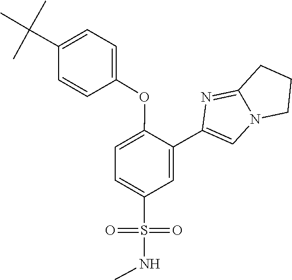 [SMILES: CNS(=O)(=O)c1ccc(Oc2ccc(C(C)(C)C)cc2)c(-c2cn3c(n2)CCC3)c1] | 4-(4-tert- butylphenoxy)-3-(6,7- dihydro-5H- pyrrolo[1,2-a]imidazol- 2-yl)-N- methylbenzene-1- sulfonamide | Calc'd 426, found 426 |
| 4-4 | 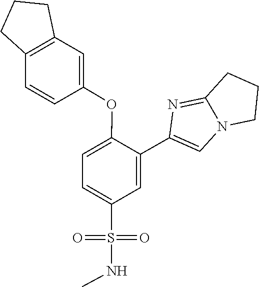 [SMILES: CNS(=O)(=O)c1ccc(Oc2ccc3c(c2)CCC3)c(-c2cn3c(n2)CCC3)c1] | 4-[(2,3-dihydro-1H- inden-5-yl)oxy]-3-(6,7- dihydro-5H- pyrrolo[1,2-a]imidazol- 2-yl)-N- methylbenzene-1- sulfonamide | Calc'd 410, found 410 |
| 4-5 | 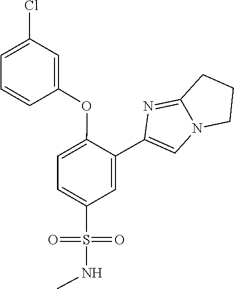 [SMILES: CNS(=O)(=O)c1ccc(Oc2cccc(Cl)c2)c(-c2cn3c(n2)CCC3)c1] | 4-(3-chlorophenoxy)-3- (6,7-dihydro-5H- pyrrolo[1,2-a]imidazol- 2-yl)-N- methylbenzene-1- sulfonamide | Calc'd 404, found 404 |
| 4-6 | 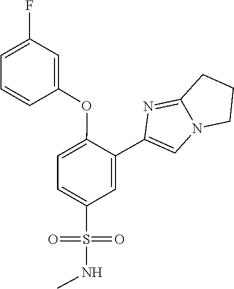 [SMILES: CNS(=O)(=O)c1ccc(Oc2cccc(F)c2)c(-c2cn3c(n2)CCC3)c1] | 3-(6,7-dihydro-5H- pyrrolo[1,2-a]imidazol- 2-yl)-4-(3- fluorophenoxy)-N- methylbenzene-1- sulfonamide | Calc'd 388, found 388 |
| 4-7 | 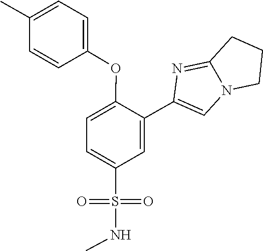 [SMILES: CNS(=O)(=O)c1ccc(Oc2ccc(C)cc2)c(-c2cn3c(n2)CCC3)c1] | 3-(6,7-dihydro-5H- pyrrolo[1,2-a]imidazol- 2-yl)-N-methyl-4-(4- methylphenoxy)benzene- 1-sulfonamide | Calc'd 384, found 384 |
| 4-8 | 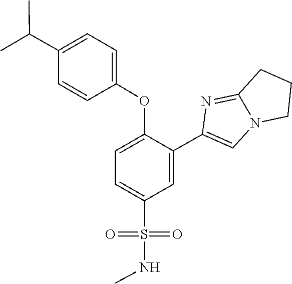 [SMILES: CNS(=O)(=O)c1ccc(Oc2ccc(C(C)C)cc2)c(-c2cn3c(n2)CCC3)c1] | 3-(6,7-dihydro-5H- pyrrolo[1,2-a]imidazol- 2-yl)-N-methyl-4-[4- (propan-2- yl)phenoxy]benzene-1- sulfonamide | Calc'd 412, found 412 |
| 4-9 | 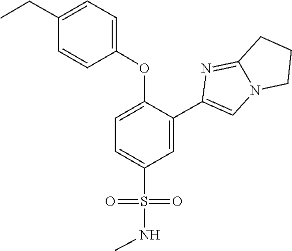 [SMILES: CCc1ccc(Oc2ccc(S(=O)(=O)NC)cc2-c2cn3c(n2)CCC3)cc1] | 3-(6,7-dihydro-5H- pyrrolo[1,2-a]imidazol- 2-yl)-4-(4- ethylphenoxy)-N- methylbenzene-1- sulfonamide | Calc'd 398, found 398 |
| 4-10 | 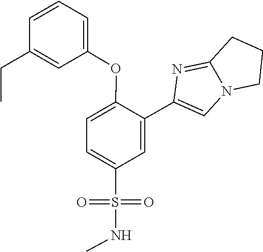 [SMILES: CCc1cccc(Oc2ccc(S(=O)(=O)NC)cc2-c2cn3c(n2)CCC3)c1] | 3-(6,7-dihydro-5H- pyrrolo[1,2-a]imidazol- 2-yl)-4-(3- ethylphenoxy)-N- methylbenzene-1- sulfonamide | Calc'd 398, found 398 |
| 4-11 | 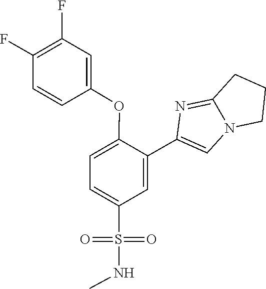 [SMILES: CNS(=O)(=O)c1ccc(Oc2ccc(F)c(F)c2)c(-c2cn3c(n2)CCC3)c1] | 4-(3,4- difluorophenoxy)-3- (6,7-dihydro-5H- pyrrolo[1,2-a]imidazol- 2-yl)-N- methylbenzene-1- sulfonamide | Calc'd 406, found 406 |
| 4-12 | 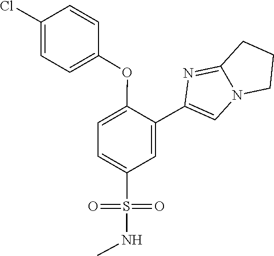 [SMILES: CNS(=O)(=O)c1ccc(Oc2ccc(Cl)cc2)c(-c2cn3c(n2)CCC3)c1] | 4-(4-chlorophenoxy)-3- (6,7-dihydro-5H- pyrrolo[1,2-a]imidazol- 2-yl)-N- methylbenzene-1- sulfonamide | Calc'd 404, found 404 |
| 4-13 | 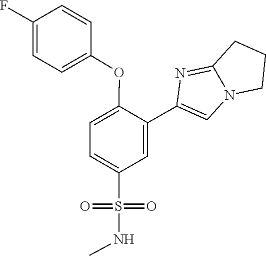 [SMILES: CNS(=O)(=O)c1ccc(Oc2ccc(F)cc2)c(-c2cn3c(n2)CCC3)c1] | 3-(6,7-dihydro-5H- pyrrolo[1,2-a]imidazol- 2-yl)-4-(4- fluorophenoxy)-N- methylbenzene-1- sulfonamide | Calc'd 388, found 388 |
| 4-14 | 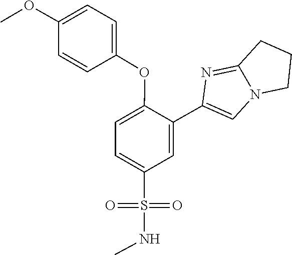 [SMILES: CNS(=O)(=O)c1ccc(Oc2ccc(OC)cc2)c(-c2cn3c(n2)CCC3)c1] | 3-(6,7-dihydro-5H- pyrrolo[1,2-a]imidazol- 2-yl)-4-(4- methoxyphenoxy)-N- methylbenzene-1- sulfonamide | Calc'd 400, found 400 |
| 4-15 | 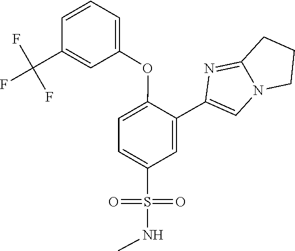 [SMILES: CNS(=O)(=O)c1ccc(Oc2cccc(C(F)(F)F)c2)c(-c2cn3c(n2)CCC3)c1] | 3-(6,7-dihydro-5H- pyrrolo[1,2-a]imidazol- 2-yl)-N-methyl-4-[3- (trifluoromethyl) phenoxy]benzene-1- sulfonamide | Calc'd 438, found 438 |
| 4-16 | 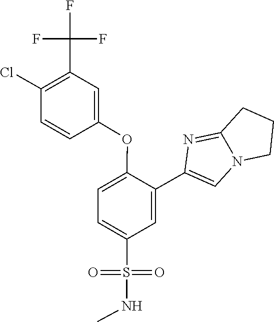 [SMILES: CNS(=O)(=O)c1ccc(Oc2ccc(Cl)c(C(F)(F)F)c2)c(-c2cn3c(n2)CCC3)c1] | 4-[4-chloro-3- (trifluoromethyl)phenoxy]- 3-(6,7-dihydro-5H- pyrrolo[1,2-a]imidazol- 2-yl)-N- methylbenzene-1- sulfonamide | Calc'd 472, found 472 |
| 4-17 | 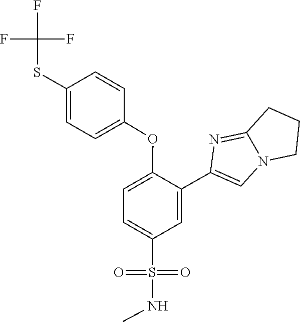 [SMILES: CNS(=O)(=O)c1ccc(Oc2ccc(SC(F)(F)F)cc2)c(-c2cn3c(n2)CCC3)c1] | 3-(6,7-dihydro-5H- pyrrolo[1,2-a]imidazol- 2-yl)-N-methyl-4-{4- [(trifluoromethyl) sulfanyl] phenoxy}benzene-1- sulfonamide | Calc'd 470, found 470 |
| 4-18 | 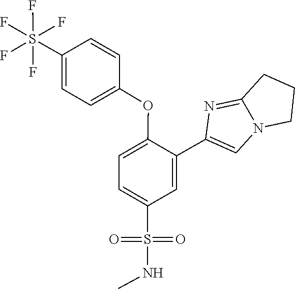 [SMILES: CNS(=O)(=O)c1ccc(Oc2ccc(S(F)(F)(F)(F)F)cc2)c(-c2cn3c(n2)CCC3)c1] | 3-(6,7-dihydro-5H- pyrrolo[1,2-a]imidazol- 2-yl)-N-methyl-4-[4- (pentafluoro- lambda~6 ~- sulfanyl)phenoxy] benzene-1-sulfonamide | Calc'd 469, found 469 |
| 4-19 | 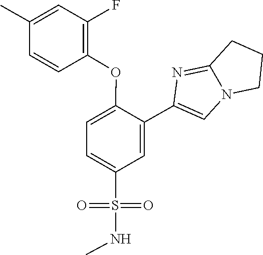 [SMILES: CNS(=O)(=O)c1ccc(Oc2ccc(C)cc2F)c(-c2cn3c(n2)CCC3)c1] | 3-(6,7-dihydro-5H- pyrrolo[1,2-a]imidazol- 2-yl)-4-(2-fluoro-4- methylphenoxy)-N- methylbenzene-1- sulfonamide | Calc'd 402, found 402 |
| 4-20 | 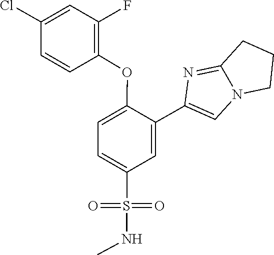 [SMILES: CNS(=O)(=O)c1ccc(Oc2ccc(Cl)cc2F)c(-c2cn3c(n2)CCC3)c1] | 4-(4-chloro-2- fluorophenoxy)-3-(6,7- dihydro-5H- pyrrolo[1,2-a]imidazol- 2-yl)-N- methylbenzene-1- sulfonamide | Calc'd 422, found 422 |
| 4-21 | 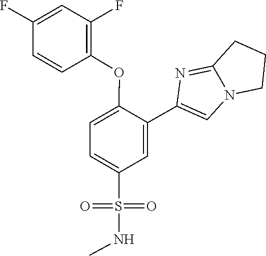 [SMILES: CNS(=O)(=O)c1ccc(Oc2ccc(F)cc2F)c(-c2cn3c(n2)CCC3)c1] | 4-(2,4- difluorophenoxy)-3- (6,7-dihydro-5H- pyrrolo[1,2-a]imidazol- 2-yl)-N- methylbenzene-1- sulfonamide | Calc'd 406, found 406 |
| 4-22 | 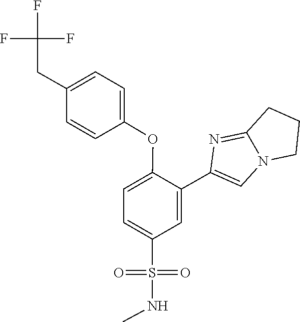 [SMILES: CNS(=O)(=O)c1ccc(Oc2ccc(CC(F)(F)F)cc2)c(-c2cn3c(n2)CCC3)c1] | 4-(2,4- difluorophenoxy)-3- (6,7-dihydro-5H- pyrrolo[1,2-a]imidazol- 2-yl)-N- methylbenzene-1- sulfonamide | Calc'd 406, found 406 |

### Example 5-1

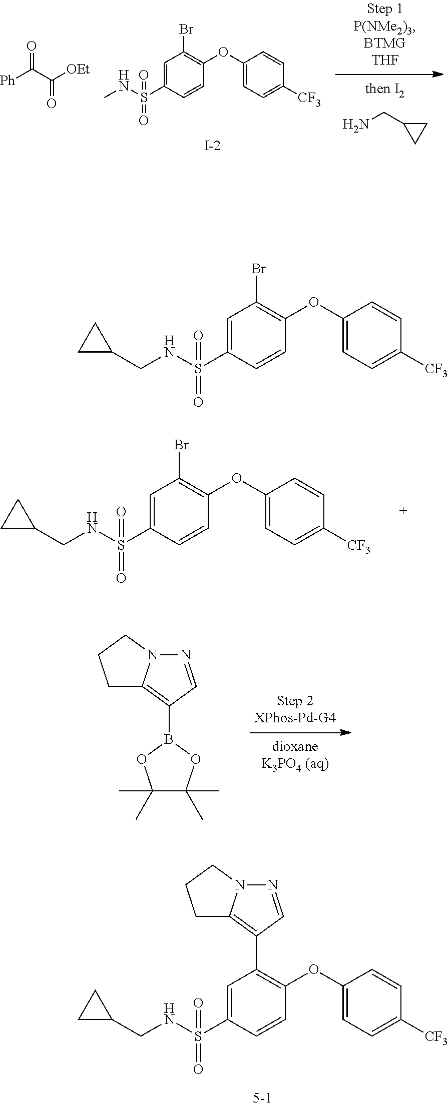

N-(Cyclopropylmethyl)-3-(5,6-dihydro-4H-pyrrolo[1,2-b]pyrazol-3-yl)-4-(4-(trifluoromethyl) phenoxy) benzenesulfonamide

Step 1. The procedure below was adapted from the following paper: Fier, P. S.; Kim, S.; Maloney, K. M. J Am. Chem. Soc. 2019, 141, 18416-18420. To 3-bromo-N-methyl-4-(4-(trifluoromethyl) phenoxy)benzenesulfonamide (I-2, 41 mg, 0.1 mmol) were added THF (0.5 mL), ethyl benzoyl formate (17 μL, 0.11 mmol), and then tris(dimethylamino)phosphine (22 μL, 0.12 mmol). The reaction vessel was purged with N₂. The solution was stirred for 1 h at RT, at which point BTMG (21 mg, 0.13 mmol) was added and the solution was subsequently heated to 65° C., then cooled to RT. To the reaction mixture was added iodine (28 mg, 0.11 mmol) and (aminomethyl)cyclopropane (21 mg, 0.3 mmol), and the resulting solution was stirred at RT overnight. The reaction mixture was diluted with EtOAc and treated with sat Na₂S₂O₃ (aq). The organic layer was separated by passage through a phase separator, and the aqueous phase was extracted with EtOAc (25 mL×2). The combined organic layers were concentrated to dryness under reduced pressure to afford 3-bromo-N-(cyclopropylmethyl)-4-(4-(trifluoromethyl)phenoxy)benzenesulfonamide. MS (ESI) m/z C₁₇H₁₆BrF₃NO₃S [M+1]⁺ calc'd 450, found 450.

Step 2. To a scintillation vial were added crude 3-bromo-N-(cyclopropylmethyl)-4-(4-(trifluoromethyl) phenoxy) benzenesulfonamide from Step 1, XPhos-Pd-G4 (9 mg, 0.01 mmol), 3-(4,4,5,5-tetramethyl-1,3,2-dioxaborolan-2-yl)-5,6-dihydro-4H-pyrrolo[1,2-b]pyrazole (26 mg, 0.11 mmol), dioxane (1 mL), and K₃PO₄ (2 M in water, 0.15 mL, 0.3 mmol). The resulting biphasic mixture was heated to 80° C. overnight, then cooled to RT. The reaction mixture was diluted with DMSO, and then filtered. The crude product solution was purified by preparative HPLC (reverse phase, C₁₈, ACN/water with 0.1% TFA modifier) to afford N-(cyclopropylmethyl)-3-(5,6-dihydro-4H-pyrrolo[1,2-b]pyrazol-3-yl)-4-(4-(trifluoromethyl)phenoxy) benzenesulfonamide, TFA. MS (ESI) m/z C₂₃H₂₃F3N₃O₃S [M+1]+ calc'd 478, found 478. ¹H NMR (600 MHz, DMSO-d₆) δ 7.88 (d, J=2.2 Hz, 1H), 7.73 (t, J=5.9 Hz, 1H), 7.71 (s, 1H), 7.69 (d, J=8.7 Hz, 2H), 7.58 (dd, J=8.5, 2.2 Hz, 1H), 7.14 (dd, J=8.6, 3.3 Hz, 3H), 4.02 (t, J=7.3 Hz, 2H), 2.95 (t, J=7.3 Hz, 2H), 2.64 (t, J=6.4 Hz, 2H), 2.46-2.40 (m, 2H), 0.81-0.71 (m, 1H), 0.34-0.27 (m, 2H), 0.04 (q, J=4.7 Hz, 2H).

### Example 6-1

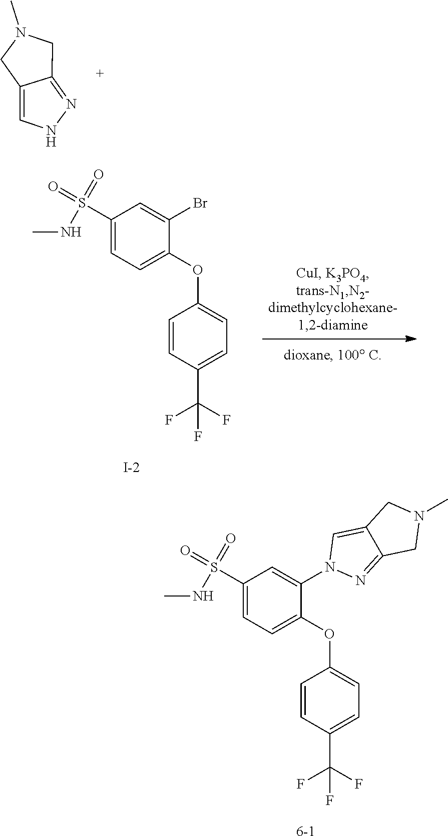

N-Methyl-3-(5-methyl-5,6-dihydropyrrolo[3,4-c]pyrazol-2(4H)-yl)-4-(4-(trifluoromethyl) phenoxy)benzenesulfonamide

To 5-methyl-2,4,5,6-tetrahydropyrrolo[3,4-c]pyrazole hydrochloride (23 mg, 0.15 mmol) was added 3-bromo-N-methyl-4-(4-(trifluoromethyl)phenoxy)benzenesulfonamide (I-2, 72 mg, 0.18 mmol), K₃PO₄ (62 mg, 0.29 mmol), and copper(I) iodide (8.4 mg, 0.044 mmol) in dry dioxane (2.44 mL). trans-N₁,N₂-Dimethylcyclohexane-1,2-diamine (9.2 μl, 0.058 mmol) was quickly added to the solution, and the mixture was degassed by sparging with N₂ for 3 min. The mixture was heated overnight at 100° C. The mixture was cooled to RT, diluted in MeOH, treated with NH₄Cl (˜15 mg) and stirred for 10 min. The mixture was diluted in MeOH and filtered. The crude product solution was purified by preparative HPLC (reverse phase, C₁₈, ACN/water with 0.1% TFA modifier) to afford N-methyl-3-(5-methyl-5,6-dihydropyrrolo[3,4-c]pyrazol-2(4H)-yl)-4-(4-(trifluoromethyl)phenoxy)benzenesulfonamide. MS (ESI) m/z C₂₀H₂₀F₃N₄O₃S [M+1]+ calc'd 453, found 453. ¹H NMR (499 MHz, DMSO-d₆) δ 11.06 (s, 1H), 8.25 (s, 1H), 8.16 (d, J=2.3 Hz, 1H), 7.84 (d, J=8.7 Hz, 2H), 7.79 (dd, J=8.7, 2.3 Hz, 1H), 7.67 (d, J=5.0 Hz, 1H), 7.38 (d, J=8.7 Hz, 2H), 4.69 (br s, 2H), 4.39-3.91 (m, 2H), 3.08 (s, 3H), 2.47 (d, J=5.0 Hz, 3H).

Compounds in Table 7 below were prepared from common Intermediate I-2 using the method described in Example 6-1.

TABLE 7

| | | | Exact |
|---|---|---|---|
| | | | Mass |
| Example | Structure | IUPAC Name | [M + H]+ |
| 6-2 | 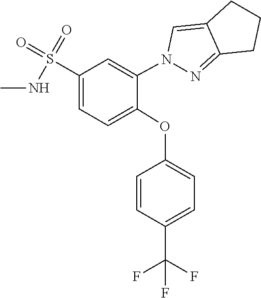 [SMILES: CNS(=O)(=O)c1ccc(Oc2ccc(C(F)(F)F)cc2)c(-n2cc3c(n2)CCC3)c1] | 3-(5,6- dihydrocyclopenta[c] pyrazol-2(4H)-yl)-N- methyl-4-[4- (trifluoromethyl) phenoxy]benzene-1- sulfonamide | Calc'd 438, found 438 |

### Example 7-1

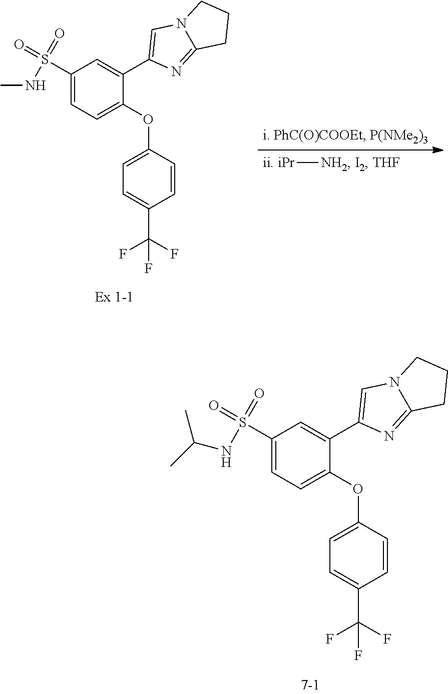

3-(6,7-Dihydro-5H-pyrrolo[1,2-α]imidazol-2-yl)-N-isopropyl-4-(4-(trifluoromethyl)phenoxy)benzenesulfonamide

A mixture of 3-(6,7-dihydro-5H-pyrrolo[1,2-α]imidazol-2-yl)-N-methyl-4-(4-(trifluoromethyl)phenoxy)benzenesulfonamide (Ex. 1-1; 100 mg, 0.23 mmol), anhydrous THF (2 mL), ethyl benzoylformate (40 uL, 0.25 mmol), and P(NMe₂)₃ (0.050 mL, 0.27 mmol) was aged at 25° C. for 30 min. Next, 2-tert-butyl-1,1,3,3-tetramethylguanidine (48.9 mg, 0.29 mmol) was added, and the resulting mixture is heated at 65° C. for 4 h. The reaction mixture was cooled to 25° C. and treated with propan-2-amine (0.059 mL, 0.69 mmol) and 12 (63.8 mg, 0.25 mmol). The resulting mixture was aged at 25° C. for 1 h. The reaction mixture was concentrated under reduced pressure and purified by preparative HPLC (reverse phase, C18, ACN/water with 0.1% TFA modifier) to afford 3-(6,7-dihydro-5H-pyrrolo[1,2-α]imidazol-2-yl)-N-isopropyl-4-(4-(trifluoromethyl)phenoxy)benzenesulfonamide. MS (ESI) m/z C₂₂H₂₃F3N₃O₃S [M+1]⁺ calc'd 466, found 466. ¹H NMR (500 MHz, CDCl₃) δ 8.63 (s, 1H), 7.83 (dd, J=2.0, 8.5 Hz, 1H), 7.70 (d, J=8.5 Hz, 2H), 7.62 (s, 1H), 7.14 (d, J=8.5 Hz, 2H), 6.86 (d, J=8.7 Hz, 1H), 5.92 (br s, 1H), 4.31 (br t, J=7.2 Hz, 2H), 3.52-3.43 (m, 1H), 3.40 (br t, J=7.7 Hz, 2H), 2.85 (br t, J=7.3 Hz, 2H), 1.08 (d, J=6.6 Hz, 6H).

Compounds in Table 8 below were prepared from Ex. 1-1 using the method described in Example 7-1.

TABLE 8

| | | | Exact |
|---|---|---|---|
| | | | Mass |
| Example | Structure | IUPAC Name | [M + H]+ |
| 7-2 | 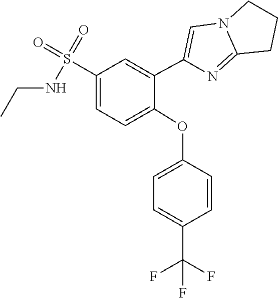 [SMILES: CCNS(=O)(=O)c1ccc(Oc2ccc(C(F)(F)F)cc2)c(-c2cn3c(n2)CCC3)c1] | 3-(6,7-dihydro- 5H-pyrrolo[1,2- a]imidazol-2-yl)- N-ethyl-4-[4- (trifluoromethyl) phenoxy]benzene- 1-sulfonamide | Calc'd 452, found 452 |
| 7-3 | 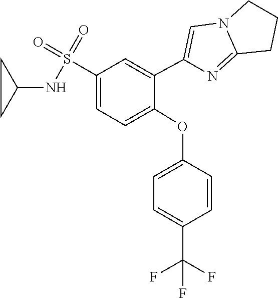 [SMILES: O=S(=O)(NC1CC1)c1ccc(Oc2ccc(C(F)(F)F)cc2)c(-c2cn3c(n2)CCC3)c1] | N-cyclopropyl- 3-(6,7-dihydro- 5H-pyrrolo[1,2- a]imidazol-2-yl)- 4-(4- (trifluoromethyl) phenoxy)benzene- sulfonamide | calc'd 464, found 464 |

### Example 8-1

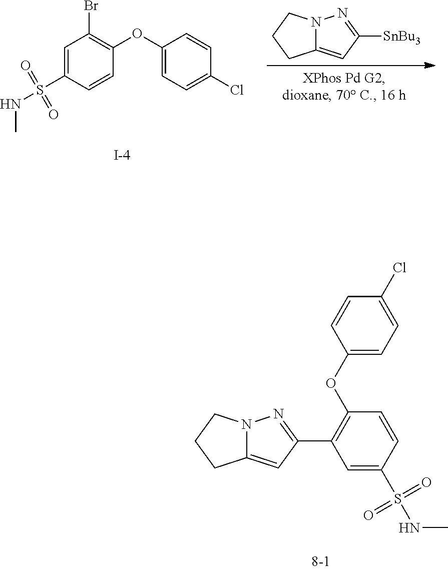

4-(4-Chlorophenoxy)-3-(5,6-dihydro-4H-pyrrolo[1,2-b]pyrazol-2-yl)-N-methylbenzene-1-sulfonamide

XPhos Pd G2 (31.3 mg, 0.040 mmol) was added to a stirred mixture of 3-bromo-4-(4-chlorophenoxy)-N-methylbenzenesulfonamide (I-4; 150 mg, 0.398 mmol) and 2-(tributylstannyl)-5,6-dihydro-4H-pyrrolo[1,2-b]pyrazole (158 mg, 0.398 mmol) in dioxane (1.5 ml) at room temperature and the mixture was heated with stirring at 70° C. for 16 h. The mixture was cooled, filtered, and the residue was purified by preparative HPLC (reverse phase C-18 column), eluting with Acetonitrile/Water+0.1% TFA, to give 4-(4-chlorophenoxy)-3-(5,6-dihydro-4H-pyrrolo[1,2-b]pyrazol-2-yl)-N-methylbenzenesulfonamide. MS (ESI) calc'd for C₁₉H₁₈ClN₃O₃S [M+H]+ calc'd 404, found 404. ¹H NMR (400 MHz, CD₃OD) δ 8.36 (d, J=2.45 Hz, 1H), 7.71 (dd, J=2.32, 8.68 Hz, 1H), 7.31-7.42 (m, 2H), 7.05 (d, J=8.80 Hz, 1H), 6.98-7.02 (m, 2H), 6.47 (s, 1H), 4.16 (t, J=7.21 Hz, 2H), 2.87-2.93 (m, 2H), 2.60-6.62 (m, 2H), 2.55 (s, 3H).

Compounds in Table 9 were prepared from common Intermediate 1-4, 1-23 bromide and I-24 bromide using the method described in Example 8-1.

TABLE 9

| | | | Exact |
|---|---|---|---|
| | | | Mass |
| Example | Structure | IUPAC Name | [M + H]+ |
| 8-2 | 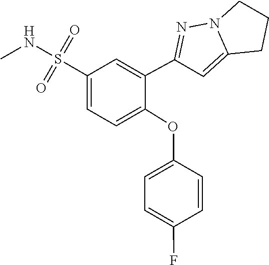 [SMILES: CNS(=O)(=O)c1ccc(Oc2ccc(F)cc2)c(-c2cc3n(n2)CCC3)c1] | 3-(5,6-dihydro-4H- pyrrolo[1,2-b]pyrazol-2- yl)-4-(4- fluorophenoxy)-N- methylbenzene-1- sulfonamide | Calc'd 388, found 388 |
| 8-3 | 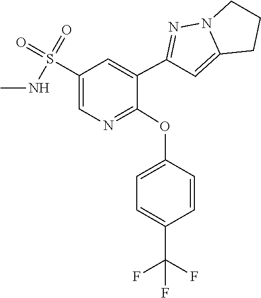 [SMILES: CNS(=O)(=O)c1cnc(Oc2ccc(C(F)(F)F)cc2)c(-c2cc3n(n2)CCC3)c1] | 5-(5,6-dihydro-4H- pyrrolo[1,2-b]pyrazol-2- yl)-N-methyl-6-[4- (trifluoromethyl) phenoxy]pyridine-3- sulfonamide | Calc'd 439, found 439 |
| 8-4 | 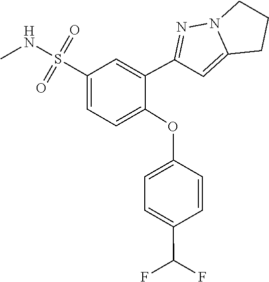 [SMILES: CNS(=O)(=O)c1ccc(Oc2ccc(C(F)F)cc2)c(-c2cc3n(n2)CCC3)c1] | 4-[4- (difluoromethyl)phenoxy]- 3-(5,6-dihydro-4H- pyrrolo[1,2-b]pyrazol-2- yl)-N-methylbenzene-1- sulfonamide | Calc'd 420, found 420 |

### Example 9-1

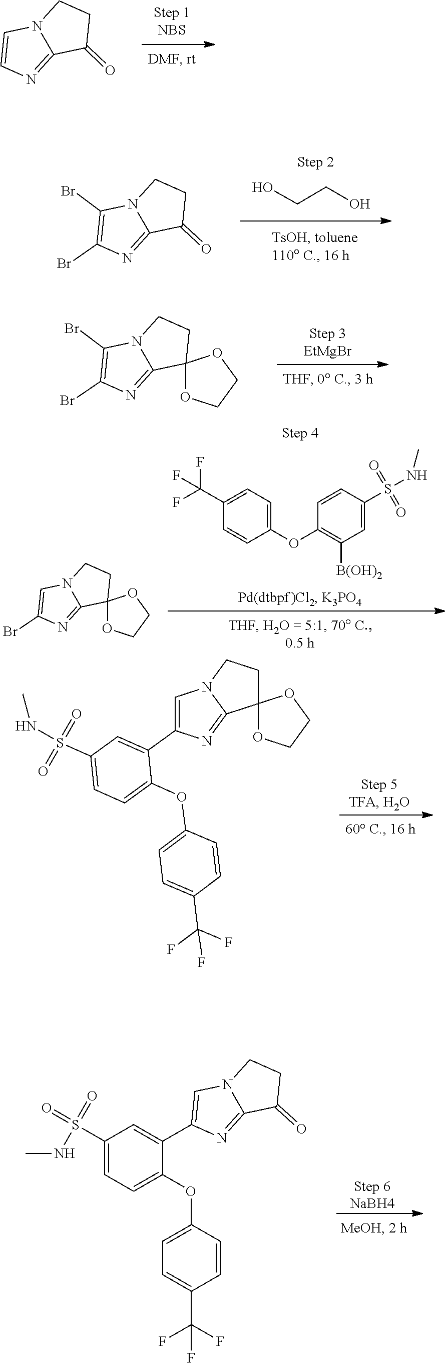

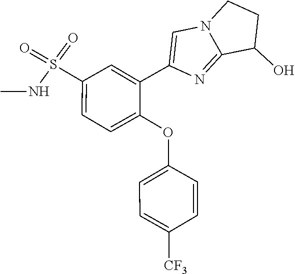 [SMILES: CNS(=O)(=O)c1ccc(Oc2ccc(C(F)(F)F)cc2)c(-c2cn3c(n2)C(O)CC3)c1]

3-(7-Hydroxy-6,7-dihydro-5H-pyrrolo[1,2-α]imidazol-2-yl)-N-methyl-4-(4-(trifluoromethyl) phenoxy)benzenesulfonamide

Step 1: To a solution of 5,6-dihydro-7H-pyrrolo[1,2-α]imidazol-7-one (0.3 g, 2.5 mmol) in DMF (3 ml) was added NBS (1.09 g, 6.14 mmol). The reaction mixture was stirred at 20° C. for 16 h. The reaction mixture was concentrated. The residue was diluted with EtOAc (30 mL) and washed with brine (10 mL×3), dried (Na₂SO₄), filtered and the solvent was evaporated under reduced pressure. The crude was purified by column chromatography (SiO₂, Pet. ether: EtOAc=0-50%) to give 2,3-dibromo-5,6-dihydro-7H-pyrrolo[1,2-α]imidazol-7-one. MS (ESI) m/z C₆H₄Br₂N₂O [M+H]⁺ calc'd 281, found 281.

Step 2: To a solution of 2,3-dibromo-5,6-dihydro-7H-pyrrolo[1,2-α]imidazol-7-one (196 mg, 0.700 mmol) in toluene (3 ml) at 20° C. were added ethane-1,2-diol (174 mg, 2.80 mmol) and 4-methylbenzenesulfonic acid (24.1 mg, 0.140 mmol). The mixture was stirred at 110° C. for 14 h. The reaction mixture was cooled and concentrated. The residue was diluted with EtOAc (20 mL) and washed with brine(5 mL×3), dried (Na₂SO₄), concentrated to give crude 2,3-dibromo-5,6-dihydrospiro[pyrrolo[1,2-a]imidazole-7,2′-[1,3]dioxolane]. MS (ESI) m/z C₈H₉Br₂N₂O₂ [M+1]⁺ calc'd 323, 325, found 323, 325.

Step 3: 2,3-Dibromo-5,6-dihydrospiro[pyrrolo[1,2-a]imidazole-7,2′-[1,3]dioxolane] (280 mg, 0.864 mmol) was dissolved in anhydrous THF (3 ml) under N₂. The mixture was cooled to 0° C., and ethylmagnesium bromide (2.02 ml, 6.05 mmol) was added dropwise to the reaction. The reaction was stirred at 0° C. for 3 h. The reaction was quenched with saturated NH₄Cl (20 mL) and extracted with EtOAc (3×8 mL). The combined organics were dried (MgSO₄) and concentrated in vacuo to give crude 2-bromo-5,6-dihydrospiro[pyrrolo[1,2-a]imidazole-7,2′-[1,3]dioxolane]. MS (ESI) m/z C₈H₁₀BrN₂O₂ [M+1]⁺ calc'd 245, found 245.

Step 4: PdCl₂(DTBPF) (26.1 mg, 0.040 mmol) was added to a stirred mixture of potassium phosphate (340 mg, 1.60 mmol), 2-bromo-5,6-dihydrospiro[pyrrolo[1,2-a]imidazole-7,2′-[1,3]dioxolane] (196 mg, 0.800 mmol) and (5-(N-methylsulfamoyl)-2-(4-(trifluoromethyl)phenoxy)phenyl)boronic acid (300 mg, 0.800 mmol) in THF (5 ml) and water (1 mL) at 25° C. under N₂ and the mixture was heated stirring at 70° C. for 0.5 h. The mixture was cooled, diluted with EtOAc (15 mL), dried (Na₂SO₄), filtered and the solvent was evaporated under reduced pressure. The crude was purified by silica gel column flash chromatography, eluting with petroleum ether/EtOAc=1:1 to afford N-methyl-3-(2-methyl-2,3-dihydroimidazo[2,1-b]oxazol-6-yl)-4-(4-(trifluoromethyl)phenoxy)benzenesulfonamide. MS (ESI) m/z C₂₂H₂₁F₃N₃O₅S [M+H]⁺ calc'd 496, found 496.

Step 5: Water (0.1 mL) was added to a stirring mixture of 3-(5,6-dihydrospiro[pyrrolo[1,2-a]imidazole-7,2′-[1,3]dioxolan]-2-yl)-N-methyl-4-(4-(trifluoromethyl)phenoxy) benzenesulfonamide (267 mg, 0.539 mmol) in TFA (3 ml) at 20° C. and the mixture was heated to 60° C. for 16 h. The reaction mixture was cooled to 20° C. and was concentrated in vacuo. The residue was diluted with EtOAc (30 mL) and washed with brine (15 mL×4) to give crude N-methyl-3-(7-oxo-6,7-dihydro-5H-pyrrolo[1,2-α]imidazol-2-yl)-4-(4-(trifluoromethyl)phenoxy) benzenesulfonamide. MS (ESI) m/z C₂₀H₁₇F₃N₃O₄S [M+1]⁺ calc'd 452, found 452. ¹H NMR (400 MHz, CDCl₃) δ 8.88 (s, 1H), 7.80 (s, 2H), 7.69 (m, 2H), 7.17 (br d, J=9.00 Hz, 2H), 7.01 (d, J=8.22 Hz, 1H), 6.60-4.62 (m, 1H), 4.43 (t, J=5.87 Hz, 2H), 3.23 (t, J=5.48 Hz, 2H), 2.73 (d, J=5.09 Hz, 3H).

Step 6: To a solution of N-methyl-3-(7-oxo-6,7-dihydro-5H-pyrrolo[1,2-α]imidazol-2-yl)-4-(4-(trifluoromethyl)phenoxy)benzenesulfonamide (40 mg, 0.089 mmol) in MeOH (2 ml) was added sodium tetrahydroborate (3.4 mg, 0.089 mmol). The reaction mixture was stirred at 28° C. for 16 h. The reaction mixture was quenched with water (10 mL) and extracted with EtOAc (3×6 mL) The combined organics were dried (MgSO₄), concentrated in vacuo and purified by prep-HPLC to give 3-(7-hydroxy-6,7-dihydro-5H-pyrrolo[1,2-α]imidazol-2-yl)-N-methyl-4-(4-(trifluoromethyl)phenoxy)benzenesulfonamide. MS (ESI) m/z C₂₀H₁₉F₃N₃O₄S [M+H]⁺ calc'd 454, found 454. ¹H NMR (400 MHz, CD₃OD) δ ppm 8.35 (s, 1H), 7.93 (s, 1H), 7.87 (dd, J=8.61, 2.35 Hz, 1H), 7.78 (d, J=8.22 Hz, 2H), 7.31 (d, J=8.22 Hz, 2H), 7.20 (d, J=8.61 Hz, 1H), 5.42 (br s, 1H), 4.41 (br s, 1H), 4.23 (m, 1H), 3.11 (br dd, J=13.1, 6.06 Hz, 1H), 2.61 (s, 3H), 2.57 (br d, J=4.70 Hz, 1H).

### Example 9-2

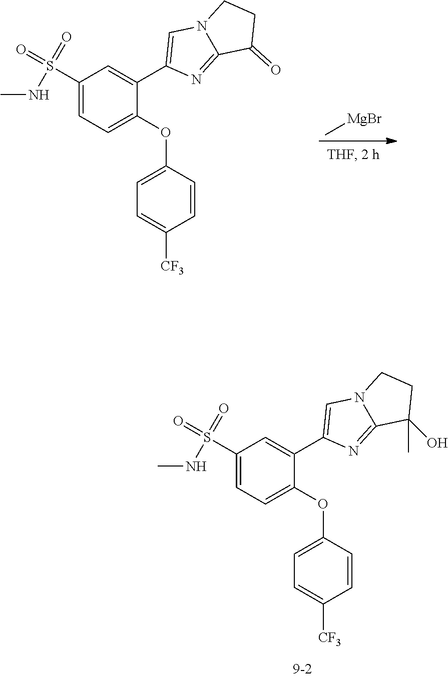

3-(7-Hydroxy-7-methyl-6,7-dihydro-5H-pyrrolo[1,2-α]imidazol-2-yl)-N-methyl-4-(4-(trifluoromethyl)phenoxy)benzenesulfonamide

To a solution of N-methyl-3-(7-oxo-6,7-dihydro-5H-pyrrolo[1,2-α]imidazol-2-yl)-4-(4-(trifluoromethyl)phenoxy)benzenesulfonamide (Ex. 9-1, Step 5; 40 mg, 0.089 mmol) in THF (4 ml) was added methylmagnesium bromide (0.089 ml, 0.266 mmol) at 0° C. The reaction mixture was stirred at 0° C. for 3 h. The reaction was quenched with NH₄Cl (5 mL) and extracted with EtOAc (3×10 mL) The combined organics were dried, concentrated and purified by prep-HPLC to give 3-(7-hydroxy-7-methyl-6,7-dihydro-5H-pyrrolo[1,2-α]imidazol-2-yl)-N-methyl-4-(4-(trifluoromethyl)phenoxy)benzenesulfonamide. MS (ESI) m/z C₂₁H₂₁F3N₃O₄S [M+H]⁺ calc'd 468, found 468. ¹H NMR (400 MHz, CD₃OD) δ 8.35 (d, J=2.35 Hz, 1H), 7.92 (s, 1H), 7.87 (dd, J=8.80, 2.15 Hz, 1H), 7.77 (d, J=8.61 Hz, 2H), 7.31 (d, J=8.61 Hz, 2H), 7.20 (d, J=9.00 Hz, 1H), 4.38-4.40 (m, 1H), 4.24-4.31 (m, 1H), 2.82-2.90 (m, 1H), 2.71-2.78 (m, 1 H), 2.60 (s, 3H), 1.77 (s, 3H).

### Example 10-1

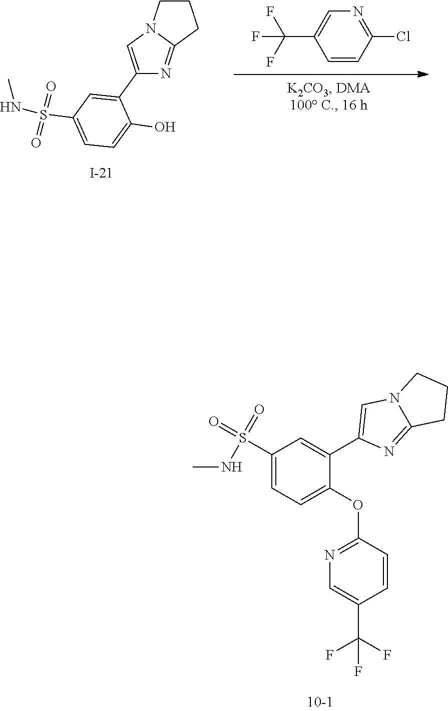

3-(6,7-Dihydro-5H-pyrrolo[1,2-α]imidazol-2-yl)-N-methyl-4-((5-(trifluoromethyl)pyridin-2-yl)oxy)benzenesulfonamide

To a solution of 3-(6,7-dihydro-5H-pyrrolo[1,2-α]imidazol-2-yl)-4-hydroxy-N-methylbenzenesulfonamide (I-21; 50.0 mg, 0.170 mmol) and 2-chloro-5-(trifluoromethyl)pyridine (46 mg, 0.26 mmol) in DMA (1 mL) was added K₂CO₃ (47 mg, 0.34 mmol) under N₂. The reaction mixture was stirred at 100° C. for 16 h. The mixture was cooled and was purified by preparative HPLC (reverse phase, C₁₈, ACN/water with 0.1% TFA modifier) to afford 3-(6,7-dihydro-5H-pyrrolo[1,2-α]imidazol-2-yl)-N-methyl-4-((5-(trifluoromethyl)pyridin-2-yl)oxy) benzenesulfonamide. MS (ESI) m/z C₁₉H₁₈F₃N₄O₃S [M+1]⁺ calc'd 439, found 439. ¹H NMR (400 MHz, CD₃OD) δ 8.49 (d, J=2.35 Hz, 1H), 8.38-8.42 (m, 1H), 8.14 (dd, J=9.00, 2.35 Hz, 1H), 7.73 (dd, J=8.61, 2.35 Hz, 1H), 7.40 (s, 1H), 7.26-7.35 (m, 1H), 3.99 (t, J=7.04 Hz, 2H), 2.82-2.87 (m, 2H), 2.62 (s, 3H), 2.53-2.61 (m, 2H).

### Example 11-1

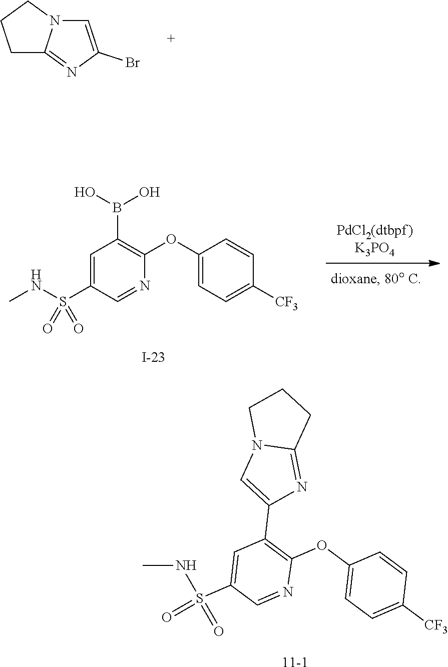

5-(6,7-Dihydro-5H-pyrrolo[1,2-α]imidazol-2-yl)-N-methyl-6-(4-(trifluoromethyl)phenoxy)pyridine-3-sulfonamide.

To 2-bromo-6,7-dihydro-5H-pyrrolo[1,2-a]imidazole (134 mg, 0.72 mmol) in dioxane (2 mL) and water (0.4 mL) was added (5-(N-methylsulfamoyl)-2-(4-(trifluoromethyl)phenoxy)pyridin-3-yl)boronic acid (I-23, 135 mg, 0.36 mmol), K₃PO₄ (229 mg, 1.08 mmol), and PdCl₂(dtbpf) (70.2 mg, 0.11 mmol). The reaction mixture was heated to 80° C. for 1 h under microwave irradiation.

The reaction was cooled to room temperature, and the crude product solution was purified by preparative HPLC (reverse phase, C18, ACN/water with 0.1% TFA modifier) to afford 5-(6,7-dihydro-5H-pyrrolo[1,2-α]imidazol-2-yl)-N-methyl-6-(4-(trifluoromethyl)phenoxy)pyridine-3-sulfonamide. MS (ESI) m/z C₁₉H₁₈F3N₄O₃S [M+1]⁺ calc'd 439, found 439. ¹H NMR (500 MHz, CD₃OD) δ 8.59 (d, J=2.5 Hz, 1H), 8.51 (d, J=2.5 Hz, 1H), 8.11 (s, 1H), 7.80 (d, J=9.0 Hz, 2H), 7.45 (d, J=8.5 Hz, 2H), 4.33 (t, J=7.3 Hz, 2H), 3.26 (t, J=7.5 Hz, 2H), 2.87-2.79 (m, 2H), 2.62 (s, 3H).

Reverse Phase Prep-HPLC Methods:

TFA Modifier

Reverse-phase preparative-HPLC [Waters SunFire OBD C18, 19 mm×150 mm(5 μm); gradient elution, ACN/H₂O/0.1% TFA]. Electrospray (ESI) Mass-triggered fraction collected was employed using positive ion polarity scanning to monitor for the target mass.

HPLC Gradient:

| Time | % | Mobile Phase Flowrate | Modifier Flowrate |
|---|---|---|---|
| (min) | Acetonitrile | (mL/Min) | (mL/min) |
| 0.0 | 2 | 25 | 0.25 |
| 3.0 | 2 | 35 | 0.35 |
| 33.0 | 95 | 35 | 0.35 |
| 33.1 | 100 | 40 | 0.4 |
| 36.1 | 100 | 50 | 0.5 |
| 36.8/end | 2 | 25 | 0.2 |

NH₄OH Modifier

Reverse-phase preparative-HPLC [Waters XBridge OBD C18, 19 mm×150 mm(5 μm); gradient elution, ACN/H₂O/0.1% NH₄₀H]. Electrospray (ESI) Mass-triggered fraction collected was employed using positive ion polarity scanning to monitor for the target mass

| Time | % | Mobile Phase Flowrate | Modifier Flowrate |
|---|---|---|---|
| (min) | Acetonitrile | (mL/Min) | (mL/min) |
| 0.0 | 2 | 25 | 0.25 |
| 3.0 | 2 | 35 | 0.35 |
| 33.0 | 95 | 35 | 0.35 |
| 33.1 | 100 | 40 | 0.4 |
| 36.1 | 100 | 50 | 0.5 |
| 36.8/end | 2 | 25 | 0.2 |

YAP/TEAD Cellular Activity Assay

Quantitation of Compound Potency on the Inhibition of TEAD-Luc Activity in TEAD Reporter-MCF7 Cell Line.

The TEAD Reporter-MCF7 cell line containing the firefly luciferase gene under the control of TEAD responsive elements stably integrated into the human breast cancer cell line was purchased from BPS Bioscience (Cat #60618). The cells were cultured with complete culture medium (EMEM 88%, 10% Non-essential amino acids, 1 mM sodium pyruvate, 10% fetal bovine serum, 10 ng/mL insulin and 400 ug/mL G418 sulfate) prior to the assay. For the assay, the cells were harvested, resuspended in the complete culture medium without G418, and seeded into white solid bottom 384-well cell culture microplates in 25 uL with 10,000 cells per well. The plates were incubated at 37° C. in a CO₂ incubator for 20-24 hours and the compounds were then transferred from Echo LDV plates directly into the 384-well white tissue culture plates with an Echo. The plates were incubated at 37° C. in a CO₂ incubator for 24 hours and equal volume of ONE-Glo™ EX Luciferase reagent (Promega, Cat #E8150) was added. After mixing on a shaker at room temperature for 10 minutes, the luciferase activity was measured with an Envision. The percentage inhibition and EC50 values of compounds were calculated with Spotfire.

Required Materials
1. TEAD reporter-MCF-7 Cells (BPS Bioscience, #60618)
2. Complete Cell Culture Medium (without selection antibiotics)
   EMEM 89% (ATCC, #30-2003)
   FBS 10% (Hyclone, #SH30088.03)
   1% Non essential amino acids (Gibco,
   1 mM sodium pyruvate (Gibco,
   Pen/Strep, 1% (Gibco, #15140-122)
   10 ng/mL insulin (Sigma, #10516)
3. Complete Cell Culture Medium (with selection antibiotics)
   EMEM 89% (ATCC, #30-2003)
   F1% Non essential amino acids (Gibco,
   1 mM sodium pyruvate (Gibco,
   BS 10% (Hyclone, #SH30088.03)
   10 ng/mL insulin (Sigma, #10516)
   400 ug/mL G418 sulfate (Agilent, #200049-21)
4. Miscellaneous tissue culture equipment and reagents.
5. White solid 384-well tissue culture plate (Greiner, Cat #781080)
6. One Glo EX luciferase assay system (Promega, #E8150)

The following table tabulates the biological data disclosed for the current invention. The biological data was collected using the methodology described above. For each compound, TEAD-Luc EC₅₀ values are listed in nanomolar (nM) concentration units.

Table of MCF7 Cellular Assay Data

| Example # | MCF7 (nM) |
|---|---|
| 1-1 | 6.0 |
| 1-2 | 5.1 |
| 1-3 | 36.2 |
| 1-4 | 6026.0 |
| 1-5 | 106.3 |
| 1-6 | 3493.0 |
| 1-7 | 5350.0 |
| 1-8 | 2868.0 |
| 1-9 | 12.8 |
| 1-10 | 454.0 |
| 1-11 | 237.6 |
| 1-12 | 5436.0 |
| 1-13 | 414.1 |
| 1-14 | 2635.0 |
| 1-15 | 540.4 |
| 1-16 | 4024.0 |
| 1-17 | 1072.0 |
| 1-18 | 34.1 |
| 1-19 | 60.6 |
| 1-20 | 7142.0 |
| 1-21 | 9280.0 |
| 1-22 | 1823.0 |
| 1-23 | 3338.0 |
| 1-24 | 5391.0 |
| 1-25 | 3272.0 |
| 1-26 | 335.4 |
| 1-27 | 28.4 |
| 1-28 | 66.6 |
| 1-29 | 860.7 |
| 1-30 | 7799 |
| 1-31 | 40.6 |
| 1-32 | 62.4 |
| 1-33 | 236.1 |
| 1-34 | 88.5 |
| 1-35 | 99.4 |
| 1-36 | 4.6 |
| 1-37 | 414 |
| 1-38 | 28.4 |
| 1-39 | 7.4 |
| 1-40 | 27 |
| 1-41 | 26.1 |
| 1-42 | 6.4 |
| 1-43 | 8.1 |
| 1-44 | 33.6 |
| 1-45 | 64.4 |
| 1-46 | 1865 |
| 1-47 | 104 |
| 1-48 | 1059 |
| 1-49 | 315 |
| 1-50 | 416 |
| 2-1 | 26.0 |
| 2-2 | 50.5 |
| 2-3 | 4920.0 |
| 2-4 | 5484.0 |
| 2-5 | 4539.0 |
| 2-6 | 210.3 |
| 2-7 | 5615.0 |
| 2-8 | 197.7 |
| 2-9 | 2184.0 |
| 2-10 | 2417.0 |
| 2-11 | 338.4 |
| 2-12 | 2863.0 |
| 2-13 | 3648.0 |
| 2-14 | 246.6 |
| 2-15 | 335.9 |
| 2-16 | 630.6 |
| 2-17 | 590.0 |
| 2-18 | 605.5 |
| 2-19 | 8855.0 |
| 2-20 | 4782.0 |
| 2-21 | 9783.0 |
| 2-22 | 92.6 |
| 2-23 | 3640.0 |
| 2-24 | 273.4 |
| 2-25 | 232.4 |
| 2-26 | 3178.0 |
| 2-27 | 409.8 |
| 2-28 | 5228.0 |
| 2-29 | 89.7 |
| 2-30 | 675.0 |
| 2-31 | 662.6 |
| 2-32 | 7574.0 |
| 2-33 | 356 |
| 3-1 | 447.1 |
| 3-2 | 9136.0 |
| 3-3 | 2082.0 |
| 3-4 | 618.5 |
| 3-5 | 3092.0 |
| 3-6 | 507.9 |
| 4-1 | 52.6 |
| 4-2 | 261 |
| 4-3 | 5.7 |
| 4-4 | 32.3 |
| 4-5 | 103 |
| 4-6 | 146 |
| 4-7 | 23.8 |
| 4-8 | 5.5 |
| 4-9 | 15.1 |
| 4-10 | 21.6 |
| 4-11 | 206 |
| 4-12 | 14.1 |
| 4-13 | 206.8 |
| 4-14 | 45.7 |
| 4-15 | 22.3 |
| 4-16 | 5.2 |
| 4-17 | 14 |
| 4-18 | 15.2 |
| 4-19 | 15.3 |
| 4-20 | 15 |
| 4-21 | 55.9 |
| 4-22 | 25.3 |
| 5-1 | 4412.0 |
| 6-1 | 6687.0 |
| 6-2 | 68.5 |
| 7-1 | 138 |
| 7-2 | 35.2 |
| 7-3 | 17.8 |
| 8-1 | 10.4 |
| 8-2 | 21.5 |
| 8-3 | 1.3 |
| 8-4 | 46.6 |
| 9-1 | 907 |
| 9-2 | 304 |
| 10-1 | 49.8 |
| 11-1 | 8.7 |

1. A compound of Formula I: 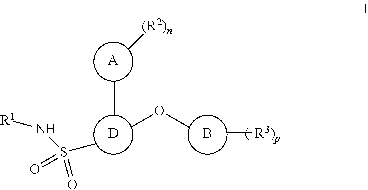
or a pharmaceutically acceptable salt thereof, wherein;
Ring A is selected from a bicyclic ring structure: 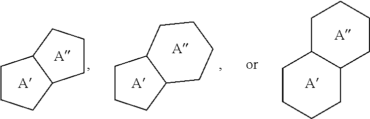
where the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring B is selected from phenyl, pyridinyl, 2,3-dihydro-1H-inden-5-yl or cyclohexyl;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R²is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆ alkyl, OR, CF₃, or halo;
where when R³ is C₄-C₆ alkyl, it can cyclize with Ring B to form a bicyclic ring;
n is selected from 0, 1, 2, 3 or 4;
p is selected from 1, 2 or 3; and
z is selected from 0, 1 or 2.

2. The compound of claim 1, or a pharmaceutically acceptable thereof, wherein:
Ring A is selected from 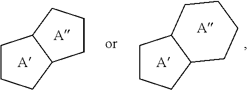 [SMILES: C1CC2CCCC2C1 ; C1CCC2CCCC2C1]
where the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring B is selected from phenyl, pyridinyl, 2,3-dihydro-1H-inden-5-yl or cyclohexyl;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R²is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆ alkyl, OR, CF₃, or halo;
n is selected from 0, 1, 2, 3 or 4;
p is selected from 1, 2 or 3; and
z is selected from 0, 1 or 2.

3. The compound of claim 1, having the structure of Formula IA 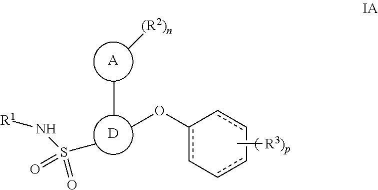
or a pharmaceutically acceptable salt thereof, wherein;
---- represents a bond that may be present or absent;
Ring A is selected from a bicyclic ring structure: 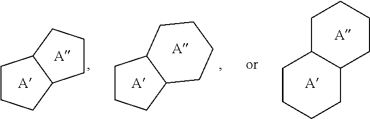
where the A′ ring may contain one or more heteroatoms selected from N, O or S atoms and the ring may be aromatic or aliphatic and the A″ ring may contain one or more heteroatoms selected from N, O or S atoms and the ring may be aromatic or aliphatic and at least one of A′ or A″ contains at least one heteroatom;
Ring D is selected from phenyl or pyridinyl;
R is independently selected from H, C₁₋₆alkyl or halo;
R¹ is selected from H, C₃-C₁₀ cycloalkyl, or C₁₋₆alkyl, wherein said alkyl is optionally substituted with one to three groups selected from C₁₋₆alkyl, C₃-C₁₀ cycloalkyl, CF₃, or (CR₂)_{z}OR;
R²is independently selected from OR, oxo, halo, —C₁₋₆alkyl, NR₂, CN, —C(O)OR, or C₃-C₁₀ cycloalkyl, where said alkyl is optionally substituted with one to three groups selected from —C₁₋₆alkyl, C₃-C₁₀ cycloalkyl, —C(O)OR, OR or halo;
R³ is independently selected from —C₁₋₆alkyl, CF₃, CHF₂, halo, OR, OCF₃, SCF₃, SF₅, or C(O)R, where said alkyl is optionally substituted with one to three groups from —C₁₋₆ alkyl, OR, CF₃, or halo;
n is selected from 0, 1, 2, 3 or 4;
p is selected from 1, 2 or 3; and
z is selected from 0, 1 or 2.

4. The compound of claim 1, or a pharmaceutically acceptable salt thereof, wherein Ring A is selected from dihydro-pyrrolo-pyrazolyl; dihydro-imidazo-oxazinyl; imidazo-pyridinyl; dihydro-pyrroloimidazolyl; imidazo-pyrazinyl; indazolyl; tetrahydroimidazo-pyridinyl; triazolopyrimidinyl; thiazolo-pyridinyl; benzimidazolyl; benzothiazolyl; dihydro-pyrrolo-thiazolyl; triazolo-pyrazinyl; tetrahydrothieno-pyridinyl; tetrahydroimidazo-pyrazinyl; imidazo-thiazolyl; pyrazolo-pyrimidinyl; dihydro-imidazo-oxazinyl; imidazo-pyridazinyl; imidazo-pyrimidinyl; benzothiazolyl; benzoxazolyl; pyrazolo-pyridinyl; tetrahydropyrazolo-pyridinyl; tetrahydroimidazo-pyridinyl; dihydroimidazo-oxazolyl; dihydro-imidazo-pyrazolyl; dihydropyrazolo-oxazolyl; cinnolinyl; dihydro-indolyl; dihydro-benzimidazolyl; indolyl; dihydro-benzimidazolyl; pyrrolo-pyridinyl; dihydro-isoindolyl; benzodioxolyl; indazolyl; dihydro-pyrazolo-oxazinyl; isoquinolinyl; quinolinyl; quinazolinyl; quinoxalinyl; dihydro-benzoxazinyl; tetrahydroquinolinyl; dihydro-benzoxazinyl; naphthyridinyl; dihydroquinazolinyl; dihydropyrazolooxazolyl; dihydropyrrolopyrazolyl; dihydrocyclopentapyrazolyl; dihydropyrrolopyrazolyl; dihydro-pyrroloimidazolyl; or azaspiroheptenyl.

5. The compound of claim 1, or a pharmaceutically acceptable salt thereof, wherein Ring A is selected from dihydropyrroloimidazolyl, dihydroimidazolyloxazinyl, dihydropyrazolooxazolyl, or dihydropyrrolopyrazolyl.

6. The compound of claim 1, or a pharmaceutically acceptable salt thereof, wherein Ring B is selected from phenyl, cyclohexyl or pyridinyl.

7. The compound of claim 1, or a pharmaceutically acceptable salt thereof, wherein R¹ is selected from C₃-C₁₀ cycloalkyl or C₁₋₆alkyl, wherein said alkyl is optionally substituted with one to three groups selected from C₁₋₆ alkyl, C₃-C₁₀ cycloalkyl, CF₃, or (CR₂)_{z}OR.

8. A compound selected from

| Ex. No. | Name |
|---|---|
| 1-1 | 3-(6,7-Dihydro-5H-pyrrolo[1,2-a]imidazol-2-yl)-N-methyl-4-(4- |
| | (trifluoromethyl)phenoxy)benzenesulfonamide |
| 1-2 | 3-(5,6-Dihydro-4H-pyrrolo[1,2-b]pyrazol-2-yl)-N-methyl-4-(4- |
| | (trifluoromethyl)phenoxy)benzenesulfonamide |
| 1-3 | 3-(5,6-Dihydro-8H-imidazo[2,1-c][1,4]oxazin-2-yl)-N-methyl- |
| | 4-(4-(trifluoromethyl)phenoxy)benzenesulfonamide |
| 1-4 | N-Methyl-3-(8-methylimidazo[1,2-a]pyridin-3-yl)-4-(4- |
| | (trifluoromethyl)phenoxy)benzenesulfonamide |
| 1-5 | 3-(5,6-dihydro-4H-pyrrolo[1,2-b]pyrazol-3-yl)-N-methyl-4-[4- |
| | (trifluoromethyl)phenoxy]benzene-1-sulfonamide |
| 1-6 | 3-(6,7-dihydro-5H-pyrrolo[1,2-a]imidazol-3-yl)-N-methyl-4-[4- |
| | (trifluoromethyl)phenoxy]benzene-1-sulfonamide |
| 1-7 | 3-(imidazo[1,5-a]pyrazin-3-yl)-N-methyl-4-[4- |
| | (trifluoromethyl)phenoxy]benzene-1-sulfonamide |
| 1-8 | 3-(2H-indazol-3-yl)-N-methyl-4-[4-(trifluoromethyl)phenoxy] |
| | benzene-1-sulfonamide |
| 1-9 | N-methyl-3-(5,6,7,8-tetrahydroimidazo[1,2-a]pyridin-2-yl)-4-[4- |
| | (trifluoromethyl)phenoxy]benzene-1-sulfonamide |
| 1-10 | N-methyl-3-([1,2,4]triazolo[1,5-a]pyrimidin-2-yl)-4-[4- |
| | (trifluoromethyl)phenoxy]benzene-1-sulfonamide |
| 1-11 | N-methyl-3-([1,3]thiazolo[5,4-b]pyridin-2-yl)-4-[4- |
| | (trifluoromethyl)phenoxy]benzene-1-sulfonamide |
| 1-12 | 3-(5-methoxy-1H-benzimidazol-2-yl)-N-methyl-4-[4- |
| | (trifluoromethyl)phenoxy]benzene-1-sulfonamide |
| 1-13 | 3-(1,2-benzothiazol-3-yl)-N-methyl-4-[4- |
| | (trifluoromethyl)phenoxy]benzene-1-sulfonamide |
| 1-14 | 3-(5,6-dihydro-4H-pyrrolo[3,4-d][1,3]thiazol-2-yl)-N-methyl- |
| | 4-[4-(trifluoromethyl)phenoxy]benzene-1-sulfonamide |
| 1-15 | N-methyl-3-([1,2,4]triazolo[1,5-a]pyrazin-2-yl)-4-[4- |
| | (trifluoromethyl)phenoxy]benzene-1-sulfonamide |
| 1-16 | N-methyl-3-(4,5,6,7-tetrahydrothieno[3,2-c]pyridin-2-yl)-4-[4- |
| | (trifluoromethyl)phenoxy]benzene-1-sulfonamide |
| 1-17 | 3-(3-cyano-5,6,7,8-tetrahydroimidazo[1,2-a]pyrazin-2-yl)-N- |
| | methyl-4-[4-(trifluoromethyl)phenoxy]benzene-1-sulfonamide |
| 1-18 | 3-(imidazo[2,1-b][1,3]thiazol-6-yl)-N-methyl-4-[4- |
| | (trifluoromethyl)phenoxy]benzene-1-sulfonamide |
| 1-19 | N-methyl-3-(pyrazolo[1,5-a]pyrimidin-2-yl)-4-[4- |
| | (trifluoromethyl)phenoxy]benzene-1-sulfonamide |
| 1-20 | 3-(imidazo[1,2-a]pyridin-3-yl)-N-methyl-4-[4- |
| | (trifluoromethyl)phenoxy]benzene-1-sulfonamide |
| 1-21 | 3-(8-aminoimidazo[1,2-a]pyridin-3-yl)-N-methyl-4-[4- |
| | (trifluoromethyl)phenoxy]benzene-1-sulfonamide |
| 1-22 | N-methyl-3-(3-methyl-5,6-dihydro-8H-imidazo[5,1-c][1,4] |
| | oxazin-1-yl)-4-[4-(trifluoromethyl)phenoxy]benzene-1- |
| | sulfonamide |
| 1-23 | 3-(6-chloroimidazo[1,2-b]pyridazin-3-yl)-N-methyl-4-[4- |
| | (trifluoromethyl)phenoxy]benzene-1-sulfonamide |
| 1-24 | 3-(6-aminoimidazo[1,2-a]pyridin-3-yl)-N-methyl-4-[4- |
| | (trifluoromethyl)phenoxy]benzene-1-sulfonamide |
| 1-25 | N-methyl-3-(7-methylimidazo[1,2-a]pyridin-3-yl)-4-[4- |
| | (trifluoromethyl)phenoxy]benzene-1-sulfonamide |
| 1-26 | 3-(imidazo[1,2-a]pyrimidin-3-yl)-N-methyl-4-[4- |
| | (trifluoromethyl)phenoxy]benzene-1-sulfonamide |
| 1-27 | 3-(imidazo[1,2-a]pyridin-2-yl)-N-methyl-4-[4- |
| | (trifluoromethyl)phenoxy]benzene-1-sulfonamide |
| 1-28 | 3-(1,3-benzothiazol-2-yl)-N-methyl-4-[4- |
| | (trifluoromethyl)phenoxy]benzene-1-sulfonamide |
| 1-29 | 3-(1,3-benzoxazol-2-yl)-N-methyl-4-[4- |
| | (trifluoromethyl)phenoxy]benzene-1-sulfonamide |
| 1-30 | Methyl-2-{5-(methylsulfamoyl)-2-[4- |
| | (trifluoromethyl)phenoxy]phenyl}imidazo[1,2-a]pyridine-7- |
| | carboxylate |
| 1-31 | N-methyl-3-(7-methylimidazo[1,2-a]pyridin-2-yl)-4-[4- |
| | (trifluoromethyl)phenoxy]benzene-1-sulfonamide |
| 1-32 | 3-(7-fluoroimidazo[1,2-a]pyridin-2-yl)-N-methyl-4-[4- |
| | (trifluoromethyl)phenoxy]benzene-1-sulfonamide |
| 1-33 | N-methyl-3-(8-methylimidazo[1,2-a]pyridin-2-yl)-4-[4- |
| | (trifluoromethyl)phenoxy]benzene-1-sulfonamide |
| 1-34 | N-methyl-3-(6-methylimidazo[1,2-a]pyridin-2-yl)-4-[4- |
| | (trifluoromethyl)phenoxy]benzene-1-sulfonamide |
| 1-35 | N-methyl-3-(pyrazolo[1,5-a]pyridin-2-yl)-4-[4- |
| | (trifluoromethyl)phenoxy]benzene-1-sulfonamide |
| 1-36 | N-methyl-3-(4,5,6,7-tetrahydropyrazolo[1,5-a]pyridin-2-yl)-4-[4- |
| | (trifluoromethyl)phenoxy]benzene-1-sulfonamide |
| 1-37 | N-methyl-3-(5,6,7,8-tetrahydroimidazo[1,5-a]pyridin-1-yl)-4-[4- |
| | (trifluoromethyl)phenoxy]benzene-1-sulfonamide |
| 1-38 | (R)- or (S)-N-methyl-3-(2-methyl-2,3-dihydroimidazo[2,1-b][1,3] |
| | oxazol-6-yl)-4-[4-(trifluoromethyl)phenoxy]benzene-1- |
| | sulfonamide |
| 1-39 | (S)- or (R)-N-methyl-3-(2-methyl-2,3-dihydroimidazo[2,1-b][1,3] |
| | oxazol-6-yl)-4-[4-(trifluoromethyl)phenoxy]benzene-1- |
| | sulfonamide |
| 1-40 | 4-[4-(difluoromethyl)phenoxy]-3-(6,7-dihydro-5H-pyrrolo[1,2- |
| | a]imidazol-2-yl)-N-methylbenzene-1-sulfonamide |
| 1-41 | (R)- or (S)-N-methyl-3-(6-methyl-6,7-dihydro-5H-pyrrolo[1,2- |
| | a]imidazol-2-yl)-4-[4-(trifluoromethyl)phenoxy]benzene-1- |
| | sulfonamide |
| 1-42 | (S)- or (R)-N-methyl-3-(6-methyl-6,7-dihydro-5H-pyrrolo[1,2- |
| | a]imidazol-2-yl)-4-[4-(trifluoromethyl)phenoxy]benzene-1- |
| | sulfonamide |
| 1-43 | (R)- or (S)-N-methyl-3-(7-methyl-6,7-dihydro-5H-pyrrolo[1,2- |
| | a]imidazol-2-yl)-4-[4-(trifluoromethyl)phenoxy]benzene-1- |
| | sulfonamide |
| 1-44 | (S)- or (R)-N-methyl-3-(7-methyl-6,7-dihydro-5H-pyrrolo[1,2- |
| | a]imidazol-2-yl)-4-[4-(trifluoromethyl)phenoxy]benzene-1- |
| | sulfonamide |
| 1-45 | 3-(2,3-dihydro-1H-imidazo[1,2-b]pyrazol-6-yl)-N-methyl-4-[4- |
| | (trifluoromethyl)phenoxy]benzene-1-sulfonamide |
| 1-46 | 3-(2,3-dihydropyrazolo[5,1-b]oxazol-7-yl)-4-(4-fluorophenoxy)- |
| | N-methylbenzenesulfonamide |
| 1-47 | 4-(4-chlorophenoxy)-3-(2,3-dihydropyrazolo[5,1-b]oxazol-7-yl)- |
| | N-methylbenzenesulfonamide |
| 1-48 | 4-(3,4-difluorophenoxy)-3-(2,3-dihydropyrazolo[5,1-b]oxazol- |
| | 7-yl)-N-methylbenzenesulfonamide |
| 1-49 | 3-(2,3-dihydropyrazolo[5,1-b]oxazol-7-yl)-N-methyl-4-(3- |
| | (trifluoromethyl)phenoxy)benzenesulfonamide |
| 1-50 | 4-(4-acetylphenoxy)-3-(6,7-dihydro-5H-pyrrolo[1,2-a]imidazol- |
| | 2-yl)-N-methylbenzene-1-sulfonamide |
| 2-1 | 3-(2,3-Dihydropyrazolo[5,1-b]oxazol-7-yl)-N-methyl-4-(4- |
| | (trifluoromethyl)phenoxy)benzenesulfonamide |
| 2-2 | 3-(3-Methoxycinnolin-7-yl)-N-methyl-4-[4- |
| | (trifluoromethyl)phenoxy]benzene-1-sulfonamide |
| 2-3 | 3-[4-methoxy-2-(trifluoromethyl)-1H-benzimidazol-7-yl]-N- |
| | methyl-4-[4-(trifluoromethyl)phenoxy]benzene-1-sulfonamide |
| 2-4 | N-methyl-3-(2-oxo-2,3-dihydro-1H-indol-5-yl)-4-[4- |
| | (trifluoromethyl)phenoxy]benzene-1-sulfonamide |
| 2-5 | N-methyl-3-(2-oxo-2,3-dihydro-1H-benzimidazol-5-yl)-4-[4- |
| | (trifluoromethyl)phenoxy]benzene-1-sulfonamide |
| 2-6 | 3-(1,3-benzothiazol-5-yl)-N-methyl-4-[4- |
| | (trifluoromethyl)phenoxy]benzene-1-sulfonamide |
| 2-7 | 3-(1H-indol-7-yl)-N-methyl-4-[4-(trifluoromethyl)phenoxy] |
| | benzene-1-sulfonamide |
| 2-8 | 3-(1,3-dimethyl-2-oxo-2,3-dihydro-1H-benzimidazol-5-yl)-N- |
| | methyl-4-[4-(trifluoromethyl)phenoxy]benzene-1-sulfonamide |
| 2-9 | N-methyl-3-(1H-pyrrolo[2,3-b]pyridin-4-yl)-4-[4- |
| | (trifluoromethyl)phenoxy]benzene-1-sulfonamide |
| 2-10 | N-methyl-3-(1H-pyrrolo[2,3-b]pyridin-5-yl)-4-[4- |
| | (trifluoromethyl)phenoxy]benzene-1-sulfonamide |
| 2-11 | 3-[2-(cyclopropylmethyl)-3-oxo-2,3-dihydro-1H-isoindol-5-yl]- |
| | N-methyl-4-[4-(trifluoromethyl)phenoxy]benzene-1-sulfonamide |
| 2-12 | 3-(1-cyclopropyl-1H-benzimidazol-6-yl)-N-methyl-4-[4- |
| | (trifluoromethyl)phenoxy]benzene-1-sulfonamide |
| 2-13 | 3-(2-amino-1H-benzimidazol-7-yl)-N-methyl-4-[4- |
| | (trifluoromethyl)phenoxy]benzene-1-sulfonamide |
| 2-14 | 3-(2H-1,3-benzodioxol-5-yl)-N-methyl-4-[4- |
| | (trifluoromethyl)phenoxy]benzene-1-sulfonamide |
| 2-15 | N-methyl-3-(1-methyl-1H-indazol-5-yl)-4-[4- |
| | (trifluoromethyl)phenoxy]benzene-1-sulfonamide |
| 2-16 | N-methyl-3-(4,5,6,7-tetrahydropyrazolo[1,5-a]pyridin-3-yl)-4-[4- |
| | (trifluoromethyl)phenoxy]benzene-1-sulfonamide |
| 2-17 | N-methyl-3-(6-methyl-6,7-dihydro-5H-pyrazolo[5,1-b][1,3] |
| | oxazin-3-yl)-4-[4-(trifluoromethyl)phenoxy]benzene-1- |
| | sulfonamide |
| 2-18 | N-methyl-3-(4-methyl-3,4-dihydro-2H-pyrido[3,2-b][1,4]oxazin- |
| | 7-yl)-4-[4-(trifluoromethyl)phenoxy]benzene-1-sulfonamide |
| 2-19 | 3-(isoquinolin-4-yl)-N-methyl-4-[4-(trifluoromethyl)phenoxy] |
| | benzene-1-sulfonamide |
| 2-20 | 3-(isoquinolin-5-yl)-N-methyl-4-[4-(trifluoromethyl)phenoxy] |
| | benzene-1-sulfonamide |
| 2-21 | 3-(7-methoxy-2-methylquinolin-6-yl)-N-methyl-4-[4- |
| | (trifluoromethyl)phenoxy]benzene-1-sulfonamide |
| 2-22 | N-methyl-3-(3-oxo-3,4-dihydro-2H-1,4-benzoxazin-6-yl)-4-[4- |
| | (trifluoromethyl)phenoxy]benzene-1-sulfonamide |
| 2-23 | 3-(7-fluoro-1-methyl-2-oxo-1,2,3,4-tetrahydroquinolin-6-yl)-N- |
| | methyl-4-[4-(trifluoromethyl)phenoxy]benzene-1-sulfonamide |
| 2-24 | N-methyl-3-(quinolin-7-yl)-4-[4-(trifluoromethyl)phenoxy] |
| | benzene-1-sulfonamide |
| 2-25 | N-methyl-3-[2-(methylamino)quinazolin-6-yl]-4-[4- |
| | (trifluoromethyl)phenoxy]benzene-1-sulfonamide |
| 2-26 | N-methyl-3-(3-oxo-3,4-dihydro-2H-1,4-benzoxazin-8-yl)-4-[4- |
| | (trifluoromethyl)phenoxy]benzene-1-sulfonamide |
| 2-27 | N-methyl-3-(quinoxalin-6-yl)-4-[4-(trifluoromethyl)phenoxy] |
| | benzene-1-sulfonamide |
| 2-28 | N-methyl-3-(3-oxo-3,4-dihydro-2H-1,4-benzoxazin-7-yl)-4-[4- |
| | (trifluoromethyl)phenoxy]benzene-1-sulfonamide |
| 2-29 | 3-(2-methoxyquinolin-6-yl)-N-methyl-4-[4- |
| | (trifluoromethyl)phenoxy]benzene-1-sulfonamide |
| 2-30 | N-methyl-3-(1,5-naphthyridin-3-yl)-4-[4- |
| | (trifluoromethyl)phenoxy]benzene-1-sulfonamide |
| 2-31 | N-methyl-3-(2-methylquinolin-6-yl)-4-[4- |
| | (trifluoromethyl)phenoxy]benzene-1-sulfonamide |
| 2-32 | N-methyl-3-(4-oxo-1,4-dihydroquinazolin-7-yl)-4-[4- |
| | (trifluoromethyl)phenoxy]benzene-1-sulfonamide |
| 2-33 | 4-[4-(difluoromethyl)phenoxy]-3-(2,3-dihydropyrazolo[5,1- |
| | b][1,3]oxazol-7-yl)-N-methylbenzene-1-sulfonamide |
| 3-1 | 3-(5,6-Dihydro-4H-pyrrolo[1,2-b]pyrazol-3-yl)-N-methyl-4-(4- |
| | ((trifluoromethyl)thio)phenoxy)benzenesulfonamide |
| 3-2 | 4-[3,5-bis(trifluoromethyl)phenoxy]-3-(5,6-dihydro-4H-pyrrolo |
| | [1,2-b]pyrazol-3-yl)-N-methylbenzene-1-sulfonamide |
| 3-3 | 3-(5,6-dihydro-4H-pyrrolo[1,2-b]pyrazol-3-yl)-4-[3-fluoro-5- |
| | (trifluoromethyl)phenoxy]-N-methylbenzene-1-sulfonamide |
| 3-4 | 3-(5,6-dihydro-4H-pyrrolo[1,2-b]pyrazol-3-yl)-N-methyl-4-[4- |
| | (trifluoromethoxy)phenoxy]benzene-1-sulfonamide |
| 3-5 | 3-(5,6-dihydro-4H-pyrrolo[1,2-b]pyrazol-3-yl)-N-methyl-4-[3- |
| | (trifluoromethoxy)phenoxy]benzene-1-sulfonamide |
| 3-6 | 3-(5,6-dihydro-4H-pyrrolo[1,2-b]pyrazol-3-yl)-N-methyl-4-[4- |
| | (2,2,2-trifluoroethyl)phenoxy]benzene-1-sulfonamide |
| 4-1 | 3-(6,7-Dihydro-5H-pyrrolo[1,2-a]imidazol-2-yl)-N-methyl-4- |
| | (((1r,4r)-4-(trifluoromethyl)cyclohexyl)oxy)benzenesulfonamide |
| 4-2 | 3-(6,7-dihydro-5H-pyrrolo[1,2-a]imidazol-2-yl)-N-methyl-4-((6- |
| | (trifluoromethyl)pyridin-3-yl)oxy)benzenesulfonamide |
| 4-3 | 4-(4-tert-butylphenoxy)-3-(6,7-dihydro-5H-pyrrolo[1,2-a] |
| | imidazol-2-yl)-N-methylbenzene-1-sulfonamide |
| 4-4 | 4-[(2,3-dihydro-1H-inden-5-yl)oxy]-3-(6,7-dihydro-5H-pyrrolo |
| | [1,2-a]imidazol-2-yl)-N-methylbenzene-1-sulfonamide |
| 4-5 | 4-(3-chlorophenoxy)-3-(6,7-dihydro-5H-pyrrolo[1,2-a]imidazol- |
| | 2-yl)-N-methylbenzene-1-sulfonamide |
| 4-6 | 3-(6,7-dihydro-5H-pyrrolo[1,2-a]imidazol-2-yl)-4-(3- |
| | fluorophenoxy)-N-methylbenzene-1-sulfonamide |
| 4-7 | 3-(6,7-dihydro-5H-pyrrolo[1,2-a]imidazol-2-yl)-N-methyl-4-(4- |
| | methylphenoxy)benzene-1-sulfonamide |
| 4-8 | 3-(6,7-dihydro-5H-pyrrolo[1,2-a]imidazol-2-yl)-N-methyl-4-[4- |
| | (propan-2-yl)phenoxy]benzene-1-sulfonamide |
| 4-9 | 3-(6,7-dihydro-5H-pyrrolo[1,2-a]imidazol-2-yl)-4-(4- |
| | ethylphenoxy)-N-methylbenzene-1-sulfonamide |
| 4-10 | 3-(6,7-dihydro-5H-pyrrolo[1,2-a]imidazol-2-yl)-4-(3- |
| | ethylphenoxy)-N-methylbenzene-1-sulfonamide |
| 4-11 | 4-(3,4-difluorophenoxy)-3-(6,7-dihydro-5H-pyrrolo[1,2-a] |
| | imidazol-2-yl)-N-methylbenzene-1-sulfonamide |
| 4-12 | 4-(4-chlorophenoxy)-3-(6,7-dihydro-5H-pyrrolo[1,2-a]imidazol- |
| | 2-yl)-N-methylbenzene-1-sulfonamide |
| 4-13 | 3-(6,7-dihydro-5H-pyrrolo[1,2-a]imidazol-2-yl)-4-(4- |
| | fluorophenoxy)-N-methylbenzene-1-sulfonamide |
| 4-14 | 3-(6,7-dihydro-5H-pyrrolo[1,2-a]imidazol-2-yl)-4-(4- |
| | methoxyphenoxy)-N-methylbenzene-1-sulfonamide |
| 4-15 | 3-(6,7-dihydro-5H-pyrrolo[1,2-a]imidazol-2-yl)-N-methyl-4- |
| | [3-(trifluoromethyl)phenoxy]benzene-1-sulfonamide |
| 4-16 | 4-[4-chloro-3-(trifluoromethyl)phenoxy]-3-(6,7-dihydro-5H- |
| | pyrrolo[1,2-a]imidazol-2-yl)-N-methylbenzene-1-sulfonamide |
| 4-17 | 3-(6,7-dihydro-5H-pyrrolo[1,2-a]imidazol-2-yl)-N-methyl-4-{4- |
| | [(trifluoromethyl)sulfanyl]phenoxy}benzene-1-sulfonamide |
| 4-18 | 3-(6,7-dihydro-5H-pyrrolo[1,2-a]imidazol-2-yl)-N-methyl-4-[4- |
| | (pentafluoro-lambda~6~-sulfanyl)phenoxy]benzene-1- |
| | sulfonamide |
| 4-19 | 3-(6,7-dihydro-5H-pyrrolo[1,2-a]imidazol-2-yl)-4-(2-fluoro-4- |
| | methylphenoxy)-N-methylbenzene-1-sulfonamide |
| 4-20 | 4-(4-chloro-2-fluorophenoxy)-3-(6,7-dihydro-5H-pyrrolo[1,2- |
| | a]imidazol-2-yl)-N-methylbenzene-1-sulfonamide |
| 4-21 | 4-(2,4-difluorophenoxy)-3-(6,7-dihydro-5H-pyrrolo[1,2-a] |
| | imidazol-2-yl)-N-methylbenzene-1-sulfonamide |
| 4-22 | 4-(2,4-difluorophenoxy)-3-(6,7-dihydro-5H-pyrrolo[1,2-a] |
| | imidazol-2-yl)-N-methylbenzene-1-sulfonamide |
| 5-1 | N-(Cyclopropylmethyl)-3-(5,6-dihydro-4H-pyrrolo[1,2-b] |
| | pyrazol-3-yl)-4-(4-(trifluoromethyl)phenoxy)benzenesulfona- |
| | mide |
| 6-1 | N-Methyl-3-(5-methyl-5,6-dihydropyrrolo[3,4-c]pyrazol-2(4H)- |
| | yl)-4-(4-(trifluoromethyl)phenoxy)benzenesulfonamide |
| 6-2 | 3-(5,6-dihydrocyclopenta[c]pyrazol-2(4H)-yl)-N-methyl-4-[4- |
| | (trifluoromethyl)phenoxy]benzene-1-sulfonamide |
| 7-1 | 3-(6,7-dihydro-5H-pyrrolo[1,2-a]imidazol-2-yl)-N-(propan-2-yl)- |
| | 4-[4-(trifluoromethyl)phenoxy]benzene-1-sulfonamide |
| 7-2 | 3-(6,7-dihydro-5H-pyrrolo[1,2-a]imidazol-2-yl)-N-ethyl-4-[4- |
| | (trifluoromethyl)phenoxy]benzene-1-sulfonamide |
| 7-3 | N-cyclopropyl-3-(6,7-dihydro-5H-pyrrolo[1,2-a]imidazol-2-yl)- |
| | 4-(4-(trifluoromethyl)phenoxy)benzenesulfonamide |
| 8-1 | 4-(4-Chlorophenoxy)-3-(5,6-dihydro-4H-pyrrolo[1,2-b]pyrazol- |
| | 2-yl)-N-methylbenzene-1-sulfonamide |
| 8-2 | 3-(5,6-dihydro-4H-pyrrolo[1,2-b]pyrazol-2-yl)-4-(4- |
| | fluorophenoxy)-N-methylbenzene-1-sulfonamide |
| 8-3 | 5-(5,6-dihydro-4H-pyrrolo[1,2-b]pyrazol-2-yl)-N-methyl-6-[4- |
| | (trifluoromethyl)phenoxy]pyridine-3-sulfonamide |
| 8-4 | 4-[4-(difluoromethyl)phenoxy]-3-(5,6-dihydro-4H-pyrrolo[1,2- |
| | b]pyrazol-2-yl)-N-methylbenzene-1-sulfonamide |
| 9-1 | 3-(7-Hydroxy-6,7-dihydro-5H-pyrrolo[1,2-a]imidazol-2-yl)-N- |
| | methyl-4-(4-(trifluoromethyl)phenoxy)benzenesulfonamide |
| 9-2 | 3-(7-Hydroxy-7-methyl-6,7-dihydro-5H-pyrrolo[1,2-a]imidazol- |
| | 2-yl)-N-methyl-4-(4-(trifluoromethyl)phenoxy) |
| | benzenesulfonamide |
| 10-1 | 3-(6,7-Dihydro-5H-pyrrolo[1,2-a]imidazol-2-yl)-N-methyl-4-((5- |
| | (trifluoromethyl)pyridin-2-yl)oxy)benzenesulfonamide |
| 11-1 | 5-(6,7-dihydro-5H-pyrrolo[1,2-a]imidazol-2-yl)-N-methyl-6-[4- |
| | (trifluoromethyl)phenoxy]pyridine-3-sulfonamide |

or a pharmaceutically acceptable salt thereof.

9. The compound of claim 8 selected from Ex. No. 1-1, 1-2, 1-3, 2-1, 4-1, 8-3, 10-1 and 11-1, or a pharmaceutically acceptable salt thereof.

10. The compound of claim 9 selected from Ex. No. 1-1, 1-2, 1-3, 2-1, 4-1, and 11-1, or a pharmaceutically acceptable salt thereof.

11. A pharmaceutical composition comprising a compound of claim 1, or a pharmaceutically acceptable salt thereof, and at least one pharmaceutical excipient.

12. A method for treating cancer in a patient, comprising administering to the patient a compound of claim 1, or a pharmaceutically acceptable salt thereof.

13. The method of claim 12, wherein the cancer is associated with increased YAP1 and/or TAZ expression.

14. A method for inhibiting the progress of cancer in a patient, comprising administering to the patient a compound of claim 1, or a pharmaceutically acceptable salt thereof.

15. A method of treating a disease or disorder in which Hippo pathway inhibition is beneficial comprising the step of administering to a patient in need thereof a therapeutically effective amount of a compound of claim 1, or a pharmaceutically acceptable salt thereof.

16. The method of claim 15 wherein the disease or disorder is a cellular proliferative disorder.

17. The method of claim 16 wherein the cellular proliferative disorder is cancer.

18. A use of a compound of claim 1, or a pharmaceutically acceptable salt thereof for treating cancer in a patient.

19. A use of a compound of claim 1, or a pharmaceutically acceptable salt thereof for the preparation of a medicament useful for the prevention of a cell proliferative disorder.
